(19) United States
(12) Patent Application Publication
(10) Pub. No.: US 2008/0145869 A1
(43) Pub. Date: Jun. 19, 2008

(54) Immunoassay method

(75) Inventors: Katsuhisa Ohzeki, Minami-Ashigara-shi (JP); Tadahiro Matsuno, Ashigarakami-gun (JP)

Correspondence Address: TAIYO CORPORATION, 401 HOLLAND LANE, #407, ALEXANDRIA, VA, 22314, US

(73) Assignee: FUJIFILM CORPORATION, Tokyo (JP)

(21) Appl. No.: 12/000,108

(22) Filed: Dec. 10, 2007

(30) Foreign Application Priority Data
Dec. 15, 2006 (JP) 2006-338972

(51) Int. Cl. G01N 33/53

(52) U.S. Cl. 435/7.9

(57) ABSTRACT

The present invention provides a method for immunoassaying an object to be analyzed including: causing to act, on a specimen or a sample including the specimen, a first protein (Y), which can specifically recognize the object to be analyzed in the specimen and is modified by a labeling substance, so as to form a complex including the object to be analyzed and Y; and measuring the labeling substance contained in the complex or the labeling substance contained in the free protein (Y), wherein the measurement of the labeling substance is performed by a chemiluminescent method which includes sensing the light generated by chemiluminescence using a photothermographic material including at least an organic silver salt, a reducing agent for silver ions and a photosensitive silver halide to form a latent image, and making the latent image visible by thermal development. An easy, quick, and highly precise immunoassay method is provided.

### CROSS-REFERENCE TO RELATED APPLICATION

This application claims priority under 35 USC 119 from Japanese Patent Application No. 2006-338972, the disclosure of which is incorporated by reference herein.

### BACKGROUND OF THE INVENTION

1. Field of the Invention

The present invention relates to an immunoassay method by which an object to be analyzed included in a sample is able to be detected easily, quickly, and highly precisely. More particularly, the invention relates to an immunoassay method by which chemiluminescence is detected with high precision using a photothermographic material.

2. Description of the Related Art

Among physiologically active substances or environmentally polluting substances including natural substances, toxins, hormones, and agricultural chemicals, those which act in extremely small amounts are very numerous. Therefore, as to qualitative and quantitative measurements of those substances, conventionally, an instrumental analytical method by which a highly sensitive analysis is possible has been widely used. However, an instrumental analysis method has a low specificity and, since not only is the analysis including a pretreatment process for the sample time-consuming but also since the operation thereof is troublesome, it is not convenient for the purpose of a quick and easy measurement which has been demanded in recent years. On the other hand, an immunoassay method has a high specificity, and the operation thereof is far easier than an instrumental analysis, whereby it has gradually become prevalent in the field of measurement of physiologically active substances or environmentally polluting substances. Among immunoassay methods, there are a latex aggregation method, an RIA method and an EIA method (enzyme immunoassay method). However, a latex aggregation method is not always satisfactory with respect to quickness and convenience or the detecting sensitivity of the measurement, while an RIA method needs a special environment since a radioisotope is used as a labeling substance.

An enzyme immunoassay method has been developed as an immunoassay method which has little risk to the human body and has been utilized as a measuring system for various substances. However, in a clinical chemical analysis, objects to be measured are biological samples (mostly serum, urine, etc.), and the measured data are often used for diagnosis of a disease condition or for observation of progression, whereby it is difficult to completely satisfy a demand for highly sensitive and highly precise measurement by a colorimetric method. For the purpose of satisfying such a demand, a chemiluminescent enzyme immunoassay method is proposed, for example, in Japanese Patent Application Laid-Open (JP-A) No. 2000-146968. All patents, patent publications, and non-patent literature cited in this specification are hereby expressly incorporated by reference herein. The chemiluminescent enzyme immunoassay method is a method where enzymatic activity is quantified by measuring a luminous amount released when an excited state of a chemiluminescent substance caused by a catalytic activity of an enzyme via an intermediate returns to a ground state and then an amount of the object to be measured having correlation to the enzymatic activity is quantified. In this method, there is no need for a light source because the chemiluminescent substance is made luminous by means of chemical reaction, and there is no rise in a background caused by a light source, whereby it is possible to make the measurement highly precise.

However, in the case of an object to be analyzed which grows such as a virus, detection at an earlier stage or, in other words, detection of a small amount of the virus is necessary, whereby there has been a demand for highly precise detection methods. Detection devices for measuring the luminescence are also necessary, and a simpler method has been demanded.

### SUMMARY OF THE INVENTION

The present invention has been made in view of the above circumstances and provides an immunoassay method with the following aspects.

A first aspect of the invention is to provide a method for immunoassaying an object to be analyzed comprising: causing to act, on a specimen or a sample comprising the specimen, a first protein (Y), which is able to specifically recognize the object to be analyzed in the specimen and is modified by a labeling substance, so as to form a complex including the object to be analyzed and Y; and measuring the labeling substance contained in the produced complex or the labeling substance contained in the free protein (Y), wherein the measurement of the labeling substance is carried out by a chemiluminescent method which comprises sensing the light generated by chemiluminescence using a photothermographic material comprising at least an organic silver salt, a reducing agent for silver ions and a photosensitive silver halide to form a latent image, and making the latent image visible by thermal development.

A second aspect of the invention is to provide a method for immunoassaying an object to be analyzed comprising: causing to act, on a specimen or a sample comprising the specimen, a second protein (X), which is able to specifically recognize the object to be analyzed in the specimen, and a first protein (Y), which is able to specifically recognize the object to be analyzed in the specimen and is modified by a labeling substance, simultaneously or stepwise so as to form a complex including X, the object to be analyzed and Y; and measuring the labeling substance contained in the produced complex or the labeling substance contained in the free protein (Y), wherein the measurement of the labeling substance is carried out by a chemiluminescent method which comprises sensing the light generated by chemiluminescence using a photothermographic material comprising at least an organic silver salt, a reducing agent for silver ions and a photosensitive silver halide to form a latent image, and making the latent image visible by thermal development.

A third aspect of the invention is to provide an immunoassay method comprising causing to act, on a specimen or a sample comprising the specimen, a second protein (X), which is able to specifically recognize an object to be analyzed in the specimen, and a first protein (Y), which is able to specifically recognize the object to be analyzed in the specimen and is modified by a labeling substance, simultaneously or stepwise so as to form a complex including X, the object to be analyzed and Y, wherein the labeling substance is a substance which accelerates development of a thermal developing material comprising at least an organic silver salt and a reducing agent for silver ions, and the complex is detected using the thermal developing material.

### DETAILED DESCRIPTION OF THE INVENTION

An object of the present invention is to provide an immunoassay method which is able to detect an object to be analyzed included in a sample easily, quickly, and highly precisely.

The problem of the invention described above has been solved by the following means.

The immunoassay method of the present invention is a method for immunoassaying an object to be analyzed comprising: causing to act, on a specimen or a sample including the specimen, a first protein (Y), which is able to specifically recognize the object to be analyzed in the specimen and is modified by a labeling substance, so as to form a complex including the object to be analyzed and Y; and measuring the labeling substance contained in the produced complex or the labeling substance contained in the free protein (Y), wherein the measurement of the labeling substance is carried out by a chemiluminescent method which comprises sensing the light generated by chemiluminescence using a photothermographic material comprising at least an organic silver salt, a reducing agent for silver ions and a photosensitive silver halide to form a latent image, and making the latent image visible by thermal development.

Preferably, the photothermographic material includes a nucleator.

Preferably, the photothermographic material includes a solvent for the organic silver salt.

Preferably, the organic silver salt includes a silver salt of a carboxylic acid or a silver salt of a nitrogen-containing heterocyclic compound. More preferably, a phase transition temperature of the organic silver salt is from 40° C. to 100° C.

Preferably, the labeling substance is peroxidase, microperoxidase, glucose oxidase, alkali phosphatase, β-galactosidase, or luciferase.

Preferably, in the measurement of the labeling substance, a luminol derivative, a dioxetane derivative, an acridinium derivative, a perbromic acid ester derivative, or a luciferin derivative is used as a chemiluminescent material.

Preferably, the protein which is able to specifically recognize the object to be analyzed is an antibody. More preferably, the antibody is a monoclonal antibody.

Preferably, the protein which is able to specifically recognize the object to be analyzed is carried by an insoluble carrier.

Another embodiment of the immunoassay method of the present invention is a method for immunoassaying an object to be analyzed including: causing to act, on a specimen or a sample including the specimen, a second protein (X), which is able to specifically recognize the object to be analyzed in the specimen, and a first protein (Y), which is able to specifically recognize the object to be analyzed in the specimen and is modified by a labeling substance, simultaneously or stepwise so as to form a complex including X, the object to be analyzed and Y; and measuring the labeling substance contained in the produced complex or the labeling substance contained in the free protein (Y), wherein the measurement of the labeling substance is carried out by a chemiluminescent method which comprises sensing the light generated by chemiluminescence using a photothermographic material comprising at least an organic silver salt, a reducing agent for silver ions and a photosensitive silver halide to form a latent image, and making the latent image visible by thermal development.

Preferably, the photothermographic material includes a nucleator.

Preferably, the photothermographic material includes a solvent for the organic silver salt.

Preferably, the organic silver salt includes a silver salt of a carboxylic acid or a silver salt of a nitrogen-containing heterocyclic compound. More preferably, a phase transition temperature of the organic silver salt is from 40° C. to 100° C.

Preferably, the labeling substance is peroxidase, microperoxidase, glucose oxidase, alkali phosphatase, β-galactosidase, or luciferase.

Preferably, in the measurement of the labeling substance, a luminol derivative, a dioxetane derivative, an acridinium derivative, a perbromic acid ester derivative, or a luciferin derivative is used as a chemiluminescent material.

Preferably, the protein which is able to specifically recognize the object to be analyzed is an antibody. More preferably, the antibody is a monoclonal antibody.

Preferably, the protein which is able to specifically recognize the object to be analyzed is carried by an insoluble carrier.

A third embodiment of the immunoassay method of the present invention is an immunoassay method including causing to act, on a specimen or a sample including the specimen, a second protein (X), which is able to specifically recognize an object to be analyzed in the specimen, and a first protein (Y), which is able to specifically recognize the object to be analyzed in the specimen and is modified by a labeling substance, simultaneously or stepwise so as to form a complex including X, the object to be analyzed and Y, wherein the labeling substance is a substance which accelerates development of a thermal developing material including at least an organic silver salt and a reducing agent for silver ions, and the complex is detected using the thermal developing material.

Preferably, the developing material includes a solvent for the organic silver salt.

Preferably, the organic silver salt includes a silver salt of a carboxylic acid or a silver salt of a nitrogen-containing heterocyclic compound. More preferably, a phase transition temperature of the organic silver salt is from 40° C. to 100° C.

According to the present invention, an immunoassay method which is able to detect an object to be analyzed included in a sample easily, quickly, and highly precisely is provided. In particular, an immunoassay method in which detection is carried out with high precision using a thermal developing material is provided.

According to the present invention, a chemiluminous enzyme reaction is able to be detected using a photosensitive thermal developing material. Further, detection can be performed using a non-photosensitive thermal developing material through fixing a material that accelerates development by an immunoreaction (immunoreaction development acceleration method).

According to the present invention, the light generated by chemiluminous enzyme reaction is sensed by a photothermographic material, and this is amplified by thermal development, and thereby an extremely small amount of light that is difficult to detect by conventional optical instruments can be detected with high precision. In the case of the measuring method by which detection is performed using a conventional wet silver halide photosensitive material, development using a developing solution, removal of undeveloped silver halide using a fixing solution, and the steps of water washing and drying are required, so that there exist problems of a lack of quickness and simplicity, as well as complicated steps and trouble with respect to controlling these processing solutions. Further, it is difficult to combine the step of detecting chemiluminescence by a photothermographic material and the step of wet developing processing into a single compact unit, and therefore each of these steps is independently carried out, so that there is a problem in that quickness is further lost. Moreover, there is a problem of deterioration in precision, because the information of an extremely small amount of light disappears during the period between the detection step and the developing processing step. In the immunoassay method of the present invention using a photothermographic material, only heating is performed just after detecting the light so that it is easy to combine the light detection step and the development step into a single compact unit, and therefore it is possible to improve quickness and precision. Moreover, it is possible to combine also, if necessary, the step of measuring the developed image information and transforming it to numeric values by an optical densitometer.

The chemiluminescent enzyme immunoassay method of the present invention is particularly useful as a detection method of enzyme immunoreaction.

The chemiluminescent enzyme immunoassay method of the present invention comprises an immunoreaction stage in which the object to be measured (antigen) is trapped as an enzyme-labeled immune complex through an antigen-antibody reaction; a chemiluminous reaction stage in which the formed immune complex is measured by a chemiluminescent method using the labeling enzyme that exists in the molecule; and the step of amplifying and detecting the chemiluminescence.

In each of these methods, it is possible to suitably apply known methods, and for example, methods described in the general remarks concerning immunoassay in “The Immunoassay Handbook Third edition” (Elsevier, 2005) and the literature concerning chemiluminescence in “Measurement and Application of Luminescence” (NTS Co., Ltd.; 1990) can be used.

Examples of the enzyme used for the chemiluminescent enzyme immunoassay method include peroxidase, alkali phosphatase, β-galactosidase, glucose oxidase, dehydrogenase, luciferase, and the like. Among these, alkali phosphatase and peroxidase are preferably used from the standpoints of handling easiness, easiness to obtain, and the like.

Further, in the case where various substances are quantified by labeling the antigen, nucleic acid, or the like using peroxidase as a labeling substance, although there is no particular restriction, horse-radish peroxidase (HRP) is preferably used as the peroxidase.

The compound which makes the chemiluminescent material emit light by chemical reaction is a compound which makes it possible to measure the luminous amount released when an excited state of a chemiluminescent substance caused by a catalytic activity of a labeling enzyme via an intermediate returns to a ground state. The compound is preferably a luminol derivative, a dioxetane derivative, an acridinium derivative, a perbromic acid ester derivative, or a luciferin derivative. Further, it is possible to add a chemiluminescence reinforcing agent (enhancer), and in this case, the effect of enhancing the intensity of chemiluminescence or improving persistence thereof is obtained. It is possible to perform quantification with high sensitivity in the chemiluminescence system using alkali phosphatase as the enzyme and a dioxetane derivative compound as the chemiluminescent material.

Concerning the method of antigen-antibody reaction which constitutes the immunoreaction stage, an arbitrary method can be used, and any method can be applied as long as a chemiluminous reagent, which is obtained by a reaction under light irradiation, is used.

In the invention, discrimination between the label bonded with the antibody and the free label can be performed by any means which are conventionally known. For example, a method, in which the label bonded with the antibody and the free label are separated and either of the two is taken out, and a method of performing detection in a non-separated state by utilizing the change in luminous property of the label due to bonding with the antibody are known. Examples of the means for separating the label bonded with the antibody and the free label include a centrifugal separation method utilizing difference in specific density, a sedimentation method utilizing difference in solubility, a separation method utilizing magnetism, and the like.

In the present invention, in order to make the separation easier, it is preferred that at least one of the proteins, which specifically recognize the object to be analyzed, is carried by a carrier that is insoluble with respect to the reaction solution. Examples of the insoluble carrier used for the chemiluminescent enzyme immunoassay method of the present invention include polymer compounds such as polystyrene, polyethylene, polypropylene, polyester, polyacrylonitrile, fluorocarbon resin, crosslinked dextran, polysaccharide, and the like, as well as glass, metal, magnetic particles, a combination thereof, and the like. Concerning the shape of the insoluble carrier, insoluble carriers of all sorts of shapes may be used such as, for example, tray-like, globular, fiber-like, tabular, board-like, vessel-like, or shapes of a cell, microplate, test tube, or the like.

Moreover, any methods can be used for fixing the antigen or antibody to the insoluble carrier, and a physical adsorption method, a covalent bonding method, an ion bonding method, or the like can be used.

Further, as the antibodies used in the chemiluminescent enzyme immunoassay method of the present invention, either of a monoclonal antibody or a polyclonal antibody can be used, and as the form thereof, the whole antibody or a fragment thereof such as F(ab′)₂, Fab, or the like can be used. Further, the origin of the antibody may be selected freely, but antibodies derived from mouse, rat, rabbit, sheep, goat, barndoor fowl, and the like are preferably used. It is preferred that at least one of the first protein and the second protein is monoclonal antibody, or F(ab′)₂ or F(ab′).

In the invention, the bonding of the antibody and the protein, and the chemical immunoreaction are usually carried out in a buffer solution. As the buffer solution used herein, any buffer solution such as a Tris buffer solution, phosphate buffer solution, borate buffer solution, carbonate buffer solution, glycine-sodium hydroxide buffer solution, or the like can be used. The concentration of the buffer used is preferably in a range of from 1 mM to 1 M. Additives such as a surfactant, chelating agent, or the like can be used freely in the buffer solution.

In the chemiluminescent enzyme immunoassay method of the present invention, measurement of the luminous amount of the chemiluminous reaction can be measured using an emission photometer. In this process, the starting point of the measurement of luminous amount and the accumulated time thereof are selected freely, but it is preferable to select a period when the luminous amount is stable and the concentration dependency of the luminous amount is high. For example, the starting point of the measurement is from 0 hours to one hour after mixing the chemicals, preferably from 0 minutes to 30 minutes, and particularly preferably from 0 minutes to 15 minutes; and the accumulated time of the measurement is from one second to one minute, preferably from one second to 30 seconds, and particularly preferably from one second to 10 seconds.

According to the invention, in the step where the chemiluminescence is amplified and detected, the optical information is transformed to image information using a photothermographic material. Evaluation of the obtained image is carried out by judging the image visually or by judging numerical data of optical densities of the image.

In the present invention, the detection sensitivity can be enhanced by further enhancing the amplification index by utilizing nucleation development. The nucleation development makes it possible to use the development of silver halide grains that have sensed light as a trigger to develop surrounding silver halide that has not sensed the light. According to the nucleation development, even an extremely small amount of light can be detected as an image of high density with high precision, and therefore nucleation development is particularly preferably used in the present invention. In the present invention, nucleation development is preferably carried out by using a nucleator.

Next, the immunoreaction development acceleration method is described. In the immunoreaction development acceleration method of the present invention, a thermal developing material including at least an organic silver salt and a reducing agent for silver ions is used. The organic silver salt and the reducing agent are described below. In the immunoreaction development acceleration method, the labeling substance is a substance which accelerates development, and preferably has an ability to act on a substance (substrate) which is capable of releasing a substance that forms a development center (development center-forming agent) to release a development center-forming agent. That is, by the labeling substance which is fixed with the object to be analyzed through an immunoreaction, the development center-forming agent that is released from the substrate, such as a nucleator, forms a development center in the thermal developing material so that the immunoreaction can be detected with higher sensitivity by thermal development. The protein X according to the present invention may be fixed to the surface of the thermal developing material. For example, it can be fixed to gelatin that is used for a binder of a surface layer. As the labeling method, for example, a hinge method using Fab′ having an SH group can be used.

Examples of the development center-forming agent include the compound known as a nucleator in the field of silver halide photographic chemistry.

Details of the nucleator used for the chemiluminescent enzyme immunoassay method and the immunoreaction development acceleration method are described in T. H. James, “THE THEORY OF THE PHOTOGRAPHIC PROCESS, FOURTH EDITION” (Macmillan Publishing Co., Inc., pp. 393 to 395, 1977). Specific examples thereof will be described below.

1. A compound having a cyclic or acyclic thiocarbonyl group (e.g. thiourea, dithiocarbamate, trithiocarbonate, dithioester, thioamide, rhodanine, thiohydantoin, thiosemicarbazide, a derivative thereof, etc.);

2. A compound having a cyclic or acyclic thioether group (e.g. sulfide, disulfide, polysulfide, etc.);

3. Other sulfur-containing compounds (e.g. thiosulfate, thiophosphate, compounds derived therefrom, etc.);

4. A nitrogen-containing reducing compound (e.g. hydrazine, hydrazone, amine, polyamine, cyclic amine, hydroxyamine, a derivative of a quaternary ammonium salt, etc.);

5. A reducing compound (e.g. aldehyde, sulfinic acid, enediol, a metal hydride compound, an alkyl metal, a dihydro form of an aromatic compound, an active methylene compound, etc.);

6. A metal complex (e.g. four-coordinate Ni(II) complex or Fe(II) complex having sulfur as a ligand, etc.);

7. An acetylene compound; and

8. Others (a phosphonium salt, etc.)

Preferable order of these compounds is, in succession: 4, 5, and 6 are most preferable compounds, and then 1, 2, 7, and 3 are preferable.

The formula and specific examples of the compound which is particularly preferably used in the present invention are described below:

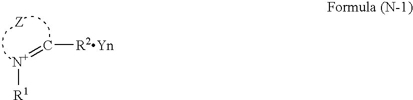

wherein Z represents a non-metallic atomic group necessary for forming a 5- or 6-membered heterocycle, and Z may be substituted by a substituent. R¹ is an aliphatic group, and R² is a hydrogen atom, an aliphatic group, or an aromatic group. R¹ and R² may be substituted by a substituent. However, at least one from among the groups represented by each of R¹, R², and Z includes an alkynyl group, an acyl group, a hydrazine group, or a hydrazone group, or R¹ and R² form a 6-membered ring to form a dihydropyridinium skeleton. Further, at least one of the substituents of R¹, R², and Z may have

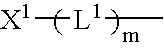

wherein X¹ is an adsorption promoting group to silver halide; and L¹ is a divalent linking group.

Y is a counter ion for balancing the electric charge; n is 0 or 1; and m is 0 or 1.

More specifically, examples of the heterocycle comprising Z include quinolinium nucleus, benzothiazolium nucleus, benzimidazolium nucleus, pyridinium nucleus, thiazolinium nucleus, thiazolium nucleus, naphthothiazolium nucleus, selenazolium nucleus, benzoselenazolium nucleus, imidazolium nucleus, tetrazolium nucleus, indolenium nucleus, pyrrolinium nucleus, acrydinium nucleus, phenanthridinium nucleus, isoquinolinium nucleus, oxazolium nucleus, naphthoxazolium nucleus, and benzoxazolium nucleus. Examples of the substituent of Z include an alkyl group, an alkenyl group, an aralkyl group, an aryl group, an alkynyl group, a hydroxy group, an alkoxy group, an aryloxy group, a halogen atom, an amino group, an alkylthio group, an arylthio group, an acyloxy group, an acylamino group, a sulfonyl group, a sulfonyloxy group, a sulfonylamino group, a carboxy group, an acyl group, a carbamoyl group, a sulfamoyl group, a sulfo group, a cyano group, a ureido group, a urethane group, a carbonic acid ester group, a hydrazine group, a hydrazone group, an imino group, and the like. At least one from among the substituents described above is selected for the substituent of Z. In the case where Z has two or more substituents, the substituents may be identical or different from each other. The substituents described above may be further substituted by these substituents.

Furthermore, the substituent of Z may include a group that forms a heterocyclic quaternary ammonium group with Z through an appropriate linking group L. In this case, the compound has a so-called dimer structure.

Preferable examples of the heterocycle comprising Z include quinolinium nucleus, benzothiazolium nucleus, benzimidazolium nucleus, pyridinium nucleus, acridinium nucleus, phenanthridinium nucleus, and isoquinolinium nucleus. More preferable are quinolinium, benzothiazolium, and benzimidazolium, even more preferable are quinolinium and benzothiazolium, and most preferable is quinolinium.

The aliphatic group represented by R¹ or R² is an unsubstituted alkyl group having 1 to 18 carbon atoms or a substituted alkyl group in which the alkyl portion has 1 to 18 carbon atoms. As the substituent, those described as the substituent of Z are described.

The aromatic group represented by R² is an aromatic group having 6 to 20 carbon atoms, and examples thereof include a phenyl group, a naphthyl group, and the like. As the substituent, those described as the substituent of Z are described.

At least one from among the groups represented by each of R¹, R², and Z has an alkynyl group, an acyl group, a hydrazine group, or a hydrazone group, or R¹ and R² form a 6-membered ring to form a dihydropyridinium skeleton. These groups may be substituted by the group which is described above as the substituent of the group represented by Z.

It is preferred that the hydrazine group has an acyl group or a sulfonyl group as the substituent.

It is preferred that the hydrazone group has an aliphatic group or an aromatic group as the substituent.

As the acyl group, for example, a formyl group and an aliphatic or aromatic ketone are preferable.

The alkynyl substituent bound to either of R¹, R², or Z has been already described above in part. In more detail, those having 2 to 18 carbon atoms are preferred, and examples thereof include an ethynyl group, a propargyl group, a 2-butynyl group, a 1-methylpropargyl group, a 1,1-dimethylpropargyl group, a 3-butynyl group, a 4-pentynyl group, and the like. The case where R¹ has a propargyl group is most preferred.

Further, these groups may be substituted by the group described above as the substituent of Z. Examples thereof include a 3-phenylpropargyl group, a 3-metboxycarbonylpropargyl group, a 4-methoxy-2-butynyl group, and the like.

The case where at least one from among the substituents of the group or ring represented by R¹ R², or Z is an alkynyl group or an acyl group, or the case where R¹ and R² link together to form a dihydropyridinium skeleton is preferred. Furthermore, the case where at least one alkynyl group is included as the substituent of the group or ring represented by R¹, R², or Z is most preferred.

Preferred examples of the adsorption promoting group to silver halide represented by X¹ include a thioamido group, a mercapto group, and a 5- or 6-membered nitrogen-containing heterocyclic group.

The thioamido adsorption promoting group represented by X¹ is a divalent group represented by

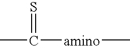

and may be a portion of a cyclic structure or may be an acyclic thioamido group. Usable thioamido adsorption promoting groups can be selected from those disclosed in U.S. Pat. Nos. 4,030,925, 4,031,127, 4,080,207, 4,245,037, 4,255,511, 4,266,013, and 4,276,364; and “Research Disclosure” vol. 151, Item 15162 (November, 1976), and ibid. vol. 176, Item 17626 (December, 1978).

Specific examples of the acyclic thioamido group include a thioureido group, a thiourethane group, a dithiocarbamic acid ester group, and the like. Specific examples of the cyclic thioamido group include 4-thiazoline-2-thione, 4-imidazoline-2-thione, 2-thiohydantoin, rhodanine, thiobarbituric acid, tetrazoline-5-thione, 1,2,4-triazoline-3-thione, 1,3,4-thiadiazoline-2-thione, 1,3,4-oxadiazoline-2-thione, benzimidazoline-2-thione, benzoxazoline-2-thione, benzothiazoline-2-thione, and the like. These groups may be further substituted.

The mercapto group represented by X¹ has the case where the —SH group is directly bonded to the group represented by R¹, R², or Z, or the case where the —SH group is bonded to the substituent of the group represented by R¹, R², or Z. Examples of the mercapto group include an aliphatic mercapto group, an aromatic mercapto group, and a heterocyclic mercapto group. (When a nitrogen atom is present next to the carbon atom to which the —SH group is bonded, the mercapto group has the same meaning as the cyclic thioamido group having a tautomeric relation thereto, specific examples of this group being the same as those enumerated above.) Examples of the 5- or 6-membered nitrogen-containing heterocyclic group represented by X¹ include 5- or 6-membered nitrogen-containing heterocycles comprising a combination of nitrogen, oxygen, sulfur, or carbon. Among these, preferable are benzotriazole, triazole, tetrazole, indazole, benzimidazole, imidazole, benzothiazole, thiazole, benzoxazole, oxazole, thiadiazole, oxadiazole, triazine, and the like. These rings may be further substituted by an appropriate substituent. As the substituent, those described as the substituent of Z are described. The nitrogen-containing heterocycle is more preferably benzotriazole, triazole, tetrazole, or indazole, and most preferably benzotriazole.

The divalent linking group represented by L¹ is an atom or atomic group each comprising at least one from among C, N, S, and O. Specific examples thereof include an alkylene group, an alkenylene group, an alkynylene group, an arylene group, —O—, —S—, —NH—, —N═, —CO—, —SO₂—, and the like (These groups may have a substituent.), and combinations thereof.

The counter ion Y for balancing the electric charge is an arbitrary anion which can cancel the positive charge due to the presence of a quaternary ammonium salt in the heterocycle. Specific examples of the counter ion Y include bromine ion, chlorine ion, iodine ion, p-toluene sulfonic acid ion, ethylsulfonic acid ion, perchloric acid ion, trifluoromethanesulfonic acid ion, thiocyan ion, and the like. In this case, n is 1. In the case where the quaternary ammonium salt includes an anionic substituent such as a sulfoalkyl substituent, the salt can take a betaine form. In such a case, counter ion is not required, and n is zero. When the quaternary ammonium salt has two anionic substituents, for example, two sulfoalkyl groups, Y is a cationic counter ion, for example, an alkali metal ion (sodium ion, potassium ion, or the like) or an ammonium salt (triethylammonium or the like).

Specific examples of the compound represented by formula (N-1) include Compounds (1) to (35) described in pages 7 to 10 of JP-A No. 1-317398.

The compounds described above can be synthesized, for example, by the methods described in patent specifications cited in “Research Disclosure” Item 22534, pp. 50 to 54 (January, 1983) or in U.S. Pat. No. 4,471,044, and similar methods thereto.

It is particularly preferred that the nucleator represented by formula (N-1) used in the present invention has an embodiment shown in the following (1) to (7), and the case of (7) is most preferred.

(1) The case where the compound has an adsorption promoting group to silver halide represented by X¹ as a substituent.

(2) The case where, in (1) described above, the adsorption promoting group to silver halide represented by X¹ comprises a thioamido group, a heterocyclic mercapto group, or a nitrogen-containing heterocycle which forms silver iminate.

(3) The case where, in (2) described above, the heterocycle comprising Z is quinolinium, isoquinolium, naphthopyridinium, or benzothiazolium.

(4) The case where, in (2) described above, the heterocycle comprising Z is quinolinium.

(5) The case where, in (2) described above, the compound has an alkynyl group as the substituent of R¹, R², or Z.

(6) The case where, in (5) described above, R¹ is a propargyl group.

(7) The case where, in (2) described above, the thioamido group for X¹ is a thiourethane group and the heterocyclic mercapto group for X¹ is mercaptotetrazole.

(8) The case where, in (6) described above, R¹ forms a ring by bonding with the heterocycle comprising Z.

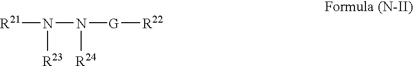

In formula (N-II) described above, R²¹ represents an aliphatic group, an aromatic group, or a heterocyclic group; R²² represents a hydrogen atom, an alkyl group, an aralkyl group, an aryl group, an alkoxy group, an aryloxy group, or an amino group; G represents a carbonyl group, a sulfonyl group, a sulfoxy group, a phosphoryl group, or an iminomethylene

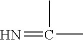

group; and both of R²³ and R²⁴ represent a hydrogen atom, or one of R²³ or R²⁴ represents a hydrogen atom and the other represents an alkylsulfonyl group, an arylsulfonyl group, or an acyl group. However, a hydrazine structure

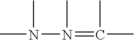

may be formed in the form including G, R²³, R²⁴, and a hydrazine nitrogen. The aforementioned groups may be further substituted by a substituent, if possible.

In formula (N-II), the aliphatic group represented by R²¹ is a straight-chain, branched, or cyclic alkyl group, alkenyl group, or alkynyl group.

The aromatic group represented by R²¹ is a monocyclic or bicyclic aryl group, and examples thereof include a phenyl group and a naphthyl group.

The heterocycle represented by R²³ is a 3- to 10-membered saturated or unsaturated heterocycle comprising at least one from among N, O, and S atom, and may be a monocycle or may form a condensed ring with other aromatic ring or heterocycle. The heterocycle is preferably a 5- or 6-membered aromatic heterocycle, for example, a pyridyl group, a quinolinyl group, an imidazolyl group, a benzimidazolyl group, or the like.

R²¹ may be substituted by a substituent. Examples of the substituent are described below. These groups may be further substituted.

Examples of the substituent include an alkyl group, an aralkyl group, an alkoxy group, an alkyl-substituted amino group, an aryl-substituted amino group, an acylamino group, a sulfonylamino group, a ureido group, a urethane group, an aryloxy group, a sulfamoyl group, a carbamoyl group, an aryl group, an alkylthio group, an arylthio group, a sulfonyl group, a sulfinyl group, a hydroxy group, a halogen atom, a cyano group, a sulfo group, a carboxyl group, etc.

These groups may link together to form a ring, if possible.

R²¹ is preferably an aromatic group, an aromatic heterocycle, or an aryl-substituted methyl group, and more preferably an aryl group.

In the case where G is a carbonyl group, the group represented by R²² is preferably a hydrogen atom, an alkyl group (for example, a methyl group, a trifluoromethyl group, a 3-hydroxypropyl group, a 3-methanesulfonamidopropyl group, or the like), an aralkyl group (for example, an o-hydroxybenzyl group or the like), or an aryl group (for example, a phenyl group, a 3,5-dichlorophenyl group, an o-methanesulfonamidophenyl group, a 4-methanesulfonylphenyl group, or the like), and particularly preferably a hydrogen atom.

In the case where G is a sulfonyl group, R²² is preferably an alkyl group (for example, a methyl group or the like), an aralkyl group (for example, an o-hydroxyphenylmethyl group or the like), an aryl group (for example, a phenyl group or the like), or a substituted amino group (for example, a dimethylamino group or the like).

As the substituent of R²², the substituents enumerated above with respect to R²¹ can be applied as well as an acyl group, an acyloxy group, an alkyloxycarbonyl group, an aryloxycarbonyl group, an alkenyl group, an alkynyl group, a nitro group, or the like can be applied.

These substituents may be further substituted by these substituents. If possible, these substituents may link together to form a ring.

It is preferred that R²¹ or R²², particularly R²¹, contains a nondiffusing group such as a coupler, a so-called ballast group. The ballast group has 8 or more carbon atoms, and comprises a combination of one or more groups selected from the group consisting of an alkyl group, a phenyl group, an ether group, an amido group, a ureido group, a urethane group, a sulfonamido group, a thioether group, and the like.

R²¹ or R²² may have a group

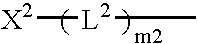

that promotes adsorption of the compound represented by formula (N-II) to silver halide grain surfaces. In the formula, X² is has the same meaning as X¹ in formula (N-I) described above, and is preferably a thioamido group (other than a thiosemicarbazide and a derivative thereof), a mercapto group, or a 5- or 6-membered nitrogen-containing heterocyclic group. L² represents a divalent linking group, and has the same as L¹ in formula (N-I) described above. m₂ is 0 or 1.

X² is more preferable a cyclic thioamido group (i.e., a mercapto-substituted nitrogen-containing heterocycle, for example, a 2-mercaptothiadiazole group, a 3-mercapto-1,2,4-triazole group, a 5-mercaptotetrazole group, a 2-mercapto-1,3,4-oxadiazole group, a 2-mercaptobenzoxazole group, or the like), or a nitrogen-containing heterocyclic group (for example, a benzotriazole group, a benzimidazole group, an indazole group, or the like).

R²³ and R²⁴ are most preferably a hydrogen atom.

G in formula (N-II) is most preferably a carbonyl group.

Further, the compound represented by formula (N-II) preferably has an adsorptive group to silver halide. The adsorptive group to silver halide is particularly preferably a mercapto group, a cyclic thioamido group, or a ureido group described above in the description on formula (N-I).

Specific examples of the compound represented by formula (N-II) include Compounds (36) to (79) described in pages 12 to 14 of JP-A No. 1-317398.

The compounds represented by formula (N-II) can be synthesized by referring to, for example, the patent specifications cited in “Research Disclosure”, Item 15162, pages 76 and 77 (November, 1976), ibid. Item 22534, pages 50 to 54 (January, 1983), and ibid. Item 23510, pages 346 to 352 (November 1983), or U.S. Pat. Nos. 4,080,207, 4,269,924, 4,273,364, 4,278,748, 4,385,108, 4,459,347, 4,478,928, and 4,560,638, British Patent No. 2,011,391B, and JP-A No. 60-179734.

It is particularly preferred that the nucleator represented by formula (N-II) described above has an embodiment shown in the following (1) to (7), and among them, the case shown in (7) is most preferred.

(1) The case where the compound has an adsorption promoting group to silver halide represented by X² as a substituent.

(2) The case where, in (1) described above, the adsorption promoting group to silver halide represented by X² is a heterocyclic mercapto group, or a nitrogen-containing heterocycle which forms silver iminate.

(3) The case where, in (2) described above, the group represented by G-R²² is a formyl group.

(4) The case where, in (3) described above, R²³ and R²⁴ are each a hydrogen atom.

(5) The case where, in (3) described above, R²¹ is an aromatic group.

(6) The case where, in (3) described above, R²¹ has a ureido group as a substituent.

(7) The case where, in (2) described above, the heterocyclic mercapto group represented by X² is 5-mercaptotetrazole or 5-mercapto-1,2,4-triazole.

Other examples of the nucleator which can be used for the present invention will be described below.

A hydrazone compound represented by the following formula:

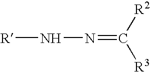

wherein R¹, R², and R³ each independently represent an alkyl group, an aryl group, a heterocyclic group, an acyl group, a sulfonyl group, an alkoxycarbonyl group, or a derivative thereof.

Specific examples thereof include hydrazone compounds described, for example, in U.S. Pat. Nos. 3,227,552 and 3,615,615, JP-A No. 52-3426, Japanese Patent Application Publication (JP-B) No. 51-1416, and the like such as 2-(2-isopropylidenehydrazino)phenyl isothiocyanate and the like.

An aldehyde compound represented by the following formula:

R—CHO

wherein R has the same meaning as R¹, R², or R³ in the above formula.

Examples thereof include aldehyde compounds described, for example, in JP-A No. 47-9678, JP-B Nos. 52-19452 and 49-20088, and the like such as the compound represented by the following formula:

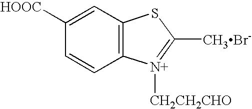

A Metal Hydride Compound

Examples thereof include metal hydride compounds described in JP-B No. 45-28065, U.S. Pat. Nos. 3,951,665 and 3,804,632, British Patent No. 821,251, and the like.

A Dihydro Compound

Further, in the present invention, dihydro compounds described, for example, in U.S. Pat. No. 3,951,656, Belgian Patent No. 708,563, German Patent Nos. 1.572,125 and 2,104,161, British Patent Nos. 1,282,084 and 1,308,753, and German Patent Application OLS No. 1,572,140 can be used.

The substrate used in the present invention is the one which is linked to at least one development center-forming agent B through at least one structure A which is to be contacted specifically by the labeling substance. Herein, the structure A and B may be linked directly or may be linked through a linking group.

The conditions required for the linkage include: (1) the enzymatic reactivity should not be inhibited by the linkage; (2) the photographic activity should not be lost by the linkage; and the like. The condition (2), i.e. the photographic activity should not be lost, includes the use of a precursor which recovers the properties at the time of contact with silver halide, at the time of development, or the like. Concerning the precursor type linking group, description can be found in JP-A Nos. 59-93442, 59-201057, 59-218439, 59-219741, 60-41034, 61-43739, and 61-95346. The linking group D may include an amino acid, a peptide, a polyamino acid, monosaccharides, disaccharides, polysaccharides(oligomer and polymer), a nucleic acid base (nucleoside, nucleotide, polynucleoside, and polynucleotide) and the like.

The linkage is carried out with a functional group on the structure A (for example, an amino group, an imino group, a carboxy group, a hydroxy group, a sulfhydryl group, a group capable of reacting with these functional groups, or the like), through a functional group on the fogging agent structure B (for example, an amino group, an imino group, a carboxy group, a hydroxy group, a sulfhydryl group, a group capable of reacting with these functional groups, or the like). These groups may previously exist in each structure or may be introduced into each structure by a chemical reaction with these groups or with a compound containing these groups. These functional groups may be employed individually or in combination therewith.

On the other hand, as the compound having a group that reacts with the aforesaid functional group, the following compounds are described: alkyl chloroformates (e.g. diethyl chloroformate, isobutyl chloroformate, etc.), aldehydes (e.g. formaldehyde, glutaraldehyde, etc.), isocyanates (e.g. xylylene diisocyanate, tolylene diisocyanate, hexamethylene diisocyanate, thioisocyanate, etc.), vinyl compounds (e.g. divinyl ketone, methylene bisacrylamide, divinyl sulfone, etc.), active halides (e.g. cyanuric chloride, mucohalogenic acids, nitrophenyl chloride, phenol-2,4-disulfonyl chloride, etc.), active esters (e.g. p-toluenesulfonic acid succinyl ester, etc.), imidazolic acid amides (e.g. carbonyl imidazole, sulfonyl diimidazole, triimidazolyl phosphate, etc.), pyridinium compounds (e.g. N-carbamoyl pyridinium, N-carbamoyloxypyridium, etc.), sulfonic acid esters (e.g. alkanesulfonic acid esters, etc.), bismaleimides (e.g. N,N′-(1,3-phenylene)bismaleimide, etc.), diazonium compounds (e.g. bisdiazobenzidine, etc.), epoxy compounds (e.g. bisoxirane, etc.), acid anhydrides, carboxylic acids, ethyleneimines, and the like.

In the above description, “fogging agent” can be changed to the wording of the development center-forming agent according to the invention.

It is preferred that the substrate according to the present invention cannot diffuse into the thermal developing material and only the compound released by the reaction with the labeling substance can diffuse into the thermal developing material. That is because, in this case, it becomes unnecessary to separate the unreacted substrate included in the reaction solution.

As the labeling substance according to the present invention, an enzyme can be used.

The enzyme which can be used in the present invention is, for example, according to the contact mode in the enzymatic reaction, a so-called hydrolase (for example, protease, nuclease, glycogenaze, esterase, lipase, or the like), which cleaves a bond such as a peptide bond, ester bond, phosphate bond, glucoside bond, acid amide bond, or the like in the substrate molecule, by addition of a water molecule; a so-called lyase and transferase, which make a specific functional group in the substrate leave or transfer to another substrate; an electron transfer-type enzyme which participates in the delivery of oxygen with the substrate or the like; a redox enzyme which participates in the redox reaction of the substrate; or the like.

Representative specific examples of the enzyme which is to be the object to be measured in the present invention include proteases such as trypsin, plasmin, kallikrein, thrombin, chymotrypsin, urokinase, catepsin, streptomycin, alkali protease, papain, ficin, bromelain, renin, collagenase, erastase, etc.; peptidoses such as leucine aminopeptidase, aminopeptidase, acylamino acid releasing enzyme, carboxypeptidase, dipeptidyl-peptidase, etc.; nucleases such as ribonuclease A, ribonuclease T₁, deoxyribonuclease A₁, endonuclease, etc.; glycogenase (including lyase) such as amylase, lysozyme, glucosidose, galactosidase, mannosidase, phosphorylase, glucanase, hyaluronidase, chondroitinase, arginic acid lyase, etc.; lipases such as lipase, phospholipase, etc.; transferases such as transcarbamylase, aminotransferase, acyltransferase, phosphotransferase, etc.; and lyases such as carboxylase, hydrolyase, ammonialyase, etc. These enzymes are described in “ENZYME”, edited by Masaru Funatsu, published by Kodansha Publishing Co., Ltd., (1977); “DATABOOK OF BIOCHEMISTRY”, The first separate volume, edited by The Japanese Biochemical Society., published by Tokyo Kagaku Dojin, (1979); “The Enzyme”, vols. III, IV, and V, edited by Paul D. Boyer et al., published by Academic Press, New York, (1971); and the like.

(Organic Silver Salt)

1) Composition

The organic silver salt which can be used in the present invention is relatively stable to light but serves to supply silver ions and forms silver images when heated to 80° C. or higher in the presence of an exposed photosensitive silver halide and a reducing agent. The organic silver salt may be any organic substance which supplies silver ions that are reducible by a reducing agent. Such a non-photosensitive organic silver salt is described, for example, in JP-A No. 10-62899 (paragraph Nos. 0048 and 0049), European Patent (EP) No. 0803764A1 (page 18, line 24 to page 19, line 37), EP No. 0962812A1, JP-A Nos. 11-349591, 2000-7683, and 2000-72711, and the like. A silver salt of an organic acid, particularly, a silver salt of a long-chain aliphatic carboxylic acid (having 10 to 30 carbon atoms, and preferably having 15 to 28 carbon atoms) is preferable. Preferred examples of the silver salt of a fatty acid include silver lignocerate, silver behenate, silver arachidinate, silver stearate, silver oleate, silver laurate, silver capronate, silver myristate, silver palmitate, silver erucate, and mixtures thereof. In the invention, among these silver salts of a fatty acid, it is preferred to use a silver salt of a fatty acid with a silver behenate content of 50 mol % or higher, more preferably 85 mol % or higher, and even more preferably 95 mol % or higher. Further, it is preferred to use a silver salt of a fatty acid with a silver erucate content of 2 mol % or lower, more preferably, 1 mol % or lower, and even more preferably, 0.1 mol % or lower.

It is preferred that the content of silver stearate is 1 mol % or lower. When the content of silver stearate is 1 mol % or lower, a silver salt of an organic acid having low fog, high sensitivity, and excellent image storability can be obtained. The above-mentioned content of silver stearate is preferably 0.5 mol % or lower, and particularly preferably, silver stearate is not substantially contained.

Further, in the case where the silver salt of an organic acid includes silver arachidinate, it is preferred that the content of silver arachidinate is 6 mol % or lower in order to obtain a silver salt of an organic acid having low fog and excellent image storability. The content of silver arachidinate is more preferably 3 mol % or lower.

In another embodiment of the present invention, the organic silver salt is a silver salt of a mercapto compound, a silver salt of a nitrogen-containing heterocyclic compound, a silver salt of an aromatic carboxylic acid, or a silver salt of a poly-carboxylic acid. Concerning the silver salt of a mercapto compound, preferred examples of the mercapto compound include an aliphatic mercapto compound and a heterocyclic mercapto compound. In the case of the aliphatic mercapto compound, the compound preferably has 10 to 30 carbon atoms, and more preferably 10 to 25 carbon atoms. The aliphatic mercapto compound may be straight-chain or branched, saturated or unsaturated, unsubstituted or substituted. In the case where the aliphatic mercapto compound has a substituent, the substituent is not particularly limited, but an alkyl group is preferred.

Preferred aliphatic group for the aliphatic mercapto compound is an alkyl group, more preferably an alkyl group having 10 to 23 carbon atoms, which includes substituted or unsubstituted, straight-chain or branched case.

Representative examples of the silver salt of an aliphatic mercapto compound are described below, but the invention is not limited to these compounds. For example, there are included a silver salt of an alkylthiol compound having 10 to 25 carbon atoms, and preferably a silver salt of an alkylthiol compound having 10 to 23 carbon atoms.

In the case of a silver salt of a heterocyclic mercapto compound, preferred examples of the heterocycle include a nitrogen-containing heterocycle, a sulfur-containing heterocycle, an oxygen-containing heterocycle, and a selenium-containing heterocycle, and more preferred are a nitrogen-containing heterocycle, a sulfur-containing heterocycle, and an oxygen-containing heterocycle. Representative examples of the silver salt of a nitrogen-containing heterocyclic mercapto compound are described below, but the invention is not limited to these examples.

Silver salt of 3-mercapto-4-phenyl-1,2,4-triazole.

Silver salt of 2-mercapto-benzimidazole.

Silver salt of 2-mercapto-5-aminothiazole.

Silver salt of mercaptotriazine.

Silver salt of 2-mercaptobenzoxazole.

A silver salt of a compound described in U.S. Pat. No. 4,123,274 (Knight, et al) (for example, a silver salt of 1,2,4-mercaptothiazole derivative, a silver salt of 3-amino-5-benzylthio-1,2,4-thiazole), and a silver salt of a thione compound (for example, a silver salt of 3-(2-carboxyethyl)-4-methyl-4-thiazoline-2-thione described in U.S. Pat. No. 3,785,830 (Sullivan, et al)).

Concerning the silver salt of a nitrogen-containing heterocyclic compound, specific examples of the nitrogen-containing heterocyclic compound include, but are not limited to these examples, azoles, oxazoles, thiazoles, thiazolines, imidazoles, diazoles, pyridines, indolizines, and triazines. Among them, more preferred are indolizines, imidazoles, and azoles. As the azoles, preferable are triazole, tetrazole, and their derivatives. More preferred are benzimidazole and a derivative thereof, and benzotriazole and a derivative thereof. As the indolizines, preferable is a triazaindolizine derivative.

Further, representative examples of the nitrogen-containing heterocyclic compound include, but are not limited to these examples, 1,2,4-triazole, benzotriazole and a derivative thereof, and preferred are benzotriazole, methylbenzotriazole, and 5-chlorobenzotriazole. Further, 1H-tetrazole compounds such as phenylmercaptotetrazole described in U.S. Pat. No. 4,220,709 (de Mauriac), and imidazole and an imidazole derivative described in U.S. Pat. No. 4,260,677 (Winslow, et al) can be described, and benzimidazole and nitrobenzimidazole are preferred. As a triazaindolizine derivative, preferred is 5-methyl-7-hydroxy-1,3,5-triazaindolizine. However, the invention is not limited to the compounds.

Concerning the silver salt of an aromatic carboxylic acid, the aromatic carboxylic acid is a substituted or unsubstituted benzenecarboxylic acid, in which the substituent is not particularly limited. Preferred are benzoic acid and a derivative thereof, and salicylic acid and a derivative thereof.

The silver salt of a poly-carboxylic acid is a silver salt of a polyvalent carboxylic acid. A silver salt of a low-molecular poly-carboxylic acid is represented by formula (I).

M¹O₂C-L¹-CO₂M² Formula (I)

In formula (1), L¹ represents an alkylene group, an alkenylene group, an alkynylene group, a cycloalkylene group, an arylene group, a divalent heterocyclic group, a divalent group selected from among —C(═O)—, —O—, —S—, —S(═O)—, —S(═O)₂—, and —N(R¹)—, or a divalent complex group formed by combining these groups. L¹ may further have a substituent.

R¹ represents a hydrogen atom or a substituent. M¹ and M² each independently represent a hydrogen atom or a counter ion, and at least one of M¹ and M² represents a silver(I) ion. The compound represented by formula (I) may further have a carboxy group or a salt thereof.

Specific examples of the compound mentioned above include, but are not limited to these examples, the compounds represented by chemical formulae Nos. 2 to 16 in paragraph Nos. 0024 to 0044 of JP-A No. 2003-330139.

Preferred examples of the carboxylic acid used for forming a silver salt of a low-molecular poly-carboxylic acid include phthalic acid, isophthalic acid, terephthalic acid, malic acid, citric acid, malonic acid, succinic acid, maleic acid, fumaric acid, hemimellitic acid, trimellitic acid, trimesic acid, mellophanic acid, prehnitic acid, pyromellitic acid, oxalic acid, adipic acid, gultaric acid, pimelic acid, suberic acid, azelaic acid, sebacic acid, and naphthalenedicarboxylic acid. Among them, particularly preferred are phthalic acid, succinic acid, adipic acid, glutaric acid, and naphthalenedicarboxylic acid. With respect to plural carboxylic acids, at least one of the carboxylic acid forms a silver salt.

A silver salt of a high-molecular poly-carboxylic acid is a silver salt of a polymer including a repeating unit derived from a monomer having a carboxy group. Preferred compound is represented by formula (II).

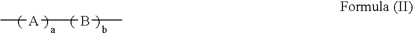

In formula (II), A represents a repeating unit derived from a monomer containing a carboxy group. B represents a repeating unit derived from an ethylenic unsaturated monomer except A a represents a number of from 5 to 100 in terms of % by weight. b represents a number of from 0 to 95 in terms of % by weight. a+b is equal to 100% by weight.

Preferably, a is a number of from 50 to 100 in terms of % by weight, b is a number of from 0 to 50 in terms of % by weight, and a+b is equal to 100% by weight.

Specifically, detailed explanation thereof is mentioned in paragraph Nos. 0013 to 0074 of JP-A No.2003-330137.

Specific examples of the carboxylic acid include the compounds described below, but are not limited to these examples. The silver salt formed with the said carboxylic acid is a silver salt of a high-molecular poly-carboxylic acid, which has at least one silver carboxylate in the molecule. In the following chemical structure, m represents a repeating number of repeating units.

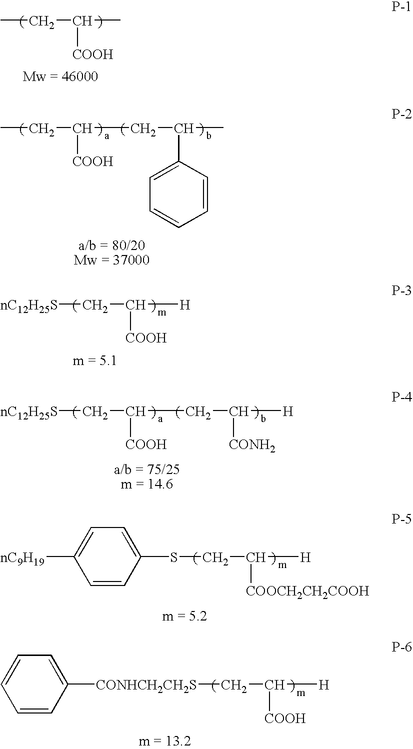

Among the organic silver salts described above, preferred examples of the silver salt of a fatty acid include silver behenate, silver stearate, silver laurate, silver oleate, silver lignocerate, and silver arachidinate. Preferred examples of the silver salt of a mercapto compound include a silver salt of 3-mercapto-4-phenyl-1,2,4-triazole, a silver salt of 2-mercapto-benzimidazole, and a silver salt of 2-mercapto-5-aminothiazole. Preferred examples of the silver salt of a nitrogen-containing heterocyclic compound include a silver salt of benzotriazole, a silver salt of methylbenzotriazole, a silver salt of benzimidazole, a silver salt of nitrobenzimidazole, and a silver salt of 5-methyl-7-hydroxy-1,3,5-triazaindolizine. Preferred examples of the silver salt of a poly-carboxylic acid include a silver salt of phthalic acid, a silver salt of succinic acid, a silver salt of adipic acid, a silver salt of glutaric acid, and a silver salt of naphthalenedicarboxylic acid. Preferred examples of the silver salt of a high-molecular poly-carboxylic acid include silver salts of P-1, P-3, and P-5 mentioned above.

Among these, particularly preferable are a silver salt of benzotriazole and a silver salt of methylbenzotriazole.

Syntheses of the silver salt of a fatty acid and the silver salt of an aliphatic mercapto compound can be carried out according to the conventional methods known in the art. For example, an aliphatic mercapto compound is melted in water by heating at a temperature above the melting point (generally, from 10° C. to 90° C. ), and then a sodium salt thereof is prepared using sodium hydroxide. Thereafter, the sodium salt is reacted with silver nitrate to obtain crystal of a silver salt of the aliphatic mercapto compound. The obtained silver salt can be dispersed using a suitable dispersing agent to prepare a dispersion thereof. In this preparing process for forming crystal of a silver salt of a fatty acid or a silver salt of an aliphatic mercapto compound, dispersion of the silver salt of a fatty acid or silver salt of an aliphatic mercapto compound may be performed in the presence of hydrophilic colloid such as gelatin. Another method for providing the silver salt comprises a step of adding a fatty acid or an aliphatic mercapto compound in a reaction vessel and thereto adding silver nitrate.

A silver salt of a heterocyclic mercapto compound and a silver salt of a low-molecular poly-carboxylic acid can be prepared similarly. As an alternative method, for example, preparation can be easily performed for technician in the art, according to the method described in “Jikken Kagaku Koza” (Lecture Series on Experimental Chemistry), 4th Ed, vol. 22, pp. 1 to 43, and pp. 193 to 227, edited by the Chemical Society of Japan, and the references cited above. A silver salt of a nitrogen-containing heterocyclic compound and a silver salt of a heterocyclic mercapto compound can be prepared by the method described in JP-A No. 1-100177. A silver salt of a high-molecular poly-carboxylic acid can also be prepared by a method similar to the above-described method.

2) Shape

There is no particular restriction on the shape of the organic silver salt that can be used in the invention, and it may be needle-like, rod-like, tabular, or flake shaped.

In the invention, a flake shaped organic silver salt is preferred. Short needle-like, rectangular, cubic, or potato-like indefinite shaped particles with a length ratio of major axis relative to minor axis being lower than 5 are also used preferably. Such organic silver salt particles suffer less from fogging during thermal development compared with long needle-like particles with the length ratio of major axis relative to minor axis being 5 or higher. Particularly, a particle with the length ratio of major axis relative to minor axis being 3 or lower is preferred since it can improve mechanical stability of the coated film. In the present specification, the flake shaped organic silver salt is defined as described below. When an organic silver salt is observed under an electron microscope, calculation is made while approximating the shape of a particle of the organic silver salt to a rectangular body, designating respective sides of the rectangular body as a, b, c from the shortest side (c may be identical with b.), and determining x based on the numeric values a and b for the shorter sides as follows.

x=b/a

In this manner, x is determined for about 200 particles, and those satisfying the relationship of x (average)≧1.5 based on an average value x are defined as flake shaped. The relationship is preferably 30≧x (average) ≧1.5, and more preferably 15≧x (average)≧1.5. Incidentally, needle-like is expressed as 1≦x (average)≦1.5.

In the flake shaped particle, a can be regarded as a thickness of a tabular particle having a major plane with b and c being as the sides. a in average is preferably from 0.01 μm to 0.3 μm and, more preferably from 0.1 μm to 0.23 μm. c/b in average is preferably from 1 to 9, more preferably from 1 to 6, even more preferably from 1 to 4 and, most preferably from 1 to 3.

By controlling the equivalent spherical diameter being from 0.05 μm to 1 μm, it causes less agglomeration in the photothermographic material and image storability is improved. The equivalent spherical diameter is preferably from 0.1 μm to 1 μm. In the invention, an equivalent spherical diameter can be measured by a method of photographing a sample directly by using an electron microscope and then image processing the negative images.

In the flake shaped particle, the equivalent spherical diameter of the particle/a is defined as an aspect ratio. The aspect ratio of the flake shaped particle is preferably from 1.1 to 30, and more preferably from 1.1 to 15 with a viewpoint of causing less agglomeration in the photothermographic material and improving the image storability.

As the particle size distribution of the organic silver salt, mono-dispersion is preferred. In the mono-dispersion, the percentage for the value obtained by dividing the standard deviation for the lengths of the minor axis and the major axis by the minor axis and the major axis respectively is preferably 100% or less, more preferably 80% or less, and even more preferably 50% or less. The shape of the organic silver salt can be measured by analyzing a dispersion of an organic silver salt as transmission type electron microscopic images. Another method of measuring the mono-dispersion is a method of determining the standard deviation of the volume-weighted mean diameter of the organic silver salt in which the percentage for the value defined by the volume-weighted mean diameter (variation coefficient) is preferably 100% or less, more preferably 80% or less, and even more preferably 50% or less. The mono-dispersion can be determined from particle size (volume-weighted mean diameter) obtained, for example, by a measuring method of irradiating a laser beam to organic silver salts dispersed in a liquid, and determining a self correlation function of the fluctuation of scattered light with respect to the change in time.

3) Preparation

Methods known in the art can be applied to the method for producing the organic silver salt used in the invention and to the dispersion method thereof. For example, reference can be made to JP-A No. 10-62899, EP Nos. 0803763A1 and 0962812A1, JP-A Nos. 11-349591, 2000-7683, 2000-72711, 2001-163889, 2001-163890, 2001-163827, 2001-33907, 2001-188313, 2001-83652, 2002-6442, 2002-49117, 2002-31870, and 2002-107868, and the like.

When a photosensitive silver salt is present together during dispersion of the organic silver salt, fog increases and sensitivity becomes remarkably lower, so that it is more preferred that the photosensitive silver salt is not substantially contained during dispersion. In the invention, the amount of the photosensitive silver salt to be dispersed in the aqueous dispersion is preferably 1 mol % or less, more preferably 0.1 mol % or less, with respect to 1 mol of the organic silver salt in the solution, and even more preferably, positive addition of the photosensitive silver salt is not conducted.

In the invention, the photothermographic material can be manufactured by mixing an aqueous dispersion of the organic silver salt and an aqueous dispersion of a photosensitive silver salt, and the mixing ratio between the organic silver salt and the photosensitive silver salt can be selected depending on the purpose. The ratio of the photosensitive silver salt relative to the organic silver salt is preferably in a range of from 1 mol % to 30 mol %, more preferably from 2 mol % to 20 mol % and, particularly preferably from 3 mol % to 15 mol %. A method of mixing two or more aqueous dispersions of organic silver salts and two or more aqueous dispersions of photosensitive silver salts upon mixing is used preferably for controlling photographic properties.

4) Addition Amount

While the organic silver salt according to the invention can be used in a desired amount, a total amount of coated silver including also the silver halide is preferably in a range of from 0.1 g/m² to 5.0 g/m², more preferably from 0.3 g/m² to 3.0 g/m², and even more preferably from 0.5 g/m² to 2.0 g/m². In particular, in order to improve image storability, the total amount of coated silver is preferably 1.8 g/m² or less, and more preferably 1.6 g/m² or less. In the case where a preferable reducing agent according to the invention is used, it is possible to obtain a sufficient image density by even such a low amount of silver.

(Reducing Agent for Silver Ions)

The photothermographic material according to the present invention may be any substance (preferably, organic substance) which reduces silver ions into metallic silver. Examples of the reducing agent are described in JP-A No. 11-65021 (paragraph Nos. 0043 to 0045) and EP No. 0803764A1 (p.7, line 34 to p. 18, line 12).

In the present invention, the reducing agent is preferably a so-called hindered phenol reducing agent or a bisphenol reducing agent having a substituent at the ortho-position with respect to the phenolic hydroxy group. It is more preferably a compound represented by the following formula (R).

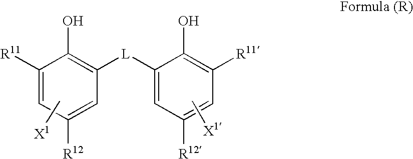

In formula (R), R¹¹ and R^{11′} each independently represent an alkyl group having 1 to 20 carbon atoms. R¹² and R^{12′} each independently represent a hydrogen atom or a substituent which substitutes for a hydrogen atom on a benzene ring. L represents an —S— group or a —CHR¹³— group. R¹³ represents a hydrogen atom or an alkyl group having 1 to 20 carbon atoms. X¹ and X^{1′} each independently represent a hydrogen atom or a group substituting for a hydrogen atom on a benzene ring.

Formula (R) is to be described in detail.

In the following description, when referred an alkyl group, it means that the alkyl group contains a cycloalkyl group, unless otherwise specified.

1) R¹¹ and R^{11′}

R¹¹ and R^{11′} each independently represent a substituted or unsubstituted alkyl group having 1 to 20 carbon atoms. The substituent of the alkyl group has no particular restriction and include, preferably, an aryl group, a hydroxy group, an alkoxy group, an aryloxy group, an alkylthio group, an arylthio group, an acylamino group, a sulfonamido group, a sulfonyl group, a phosphoryl group, an acyl group, a carbamoyl group, an ester group, a ureido group, a urethane group, a halogen atom, and the like.

2) R¹² and R^{12′}, X¹ and X^{1′}

R¹² and R^{12′} each independently represent a hydrogen atom or a substituent which substitutes for a hydrogen atom on a benzene ring. X¹ and X^{1′} each independently represent a hydrogen atom or a group substituting for a hydrogen atom on a benzene ring. As each of the groups substituting for a hydrogen atom on the benzene ring, an alkyl group, an aryl group, a halogen atom, an alkoxy group, and an acylamino group are described preferably.

3) L

L represents an —S— group or a —CHR¹³— group. R¹³ represents a hydrogen atom or an alkyl group having 1 to 20 carbon atoms in which the alkyl group may have a substituent. Specific examples of the unsubstituted alkyl group for R¹³ include a methyl group, an ethyl group, a propyl group, a butyl group, a heptyl group, an undecyl group, an isopropyl group, a 1-ethylpentyl group, a 2,4,4-trimethylpentyl group, a cyclohexyl group, a 2,4-dimethyl-3-cyclohexenyl group, a 3,5-dimethyl-3-cyclohexenyl group, and the like. Examples of the substituent of the alkyl group include, similar to the substituent of R¹¹, a halogen atom, an alkoxy group, an alkylthio group, an aryloxy group, an arylthio group, an acylamino group, a sulfonamido group, a sulfonyl group, a phosphoryl group, an oxycarbonyl group, a carbamoyl group, a sulfamoyl group, and the like. 4) Preferable Substituents

R¹¹ and R^{11′} are preferably a primary, secondary, or tertiary alkyl group having 1 to 15 carbon atoms; and examples thereof include, specifically, a methyl group, an isopropyl group, a t-butyl group, a t-amyl group, a t-octyl group, a cyclohexyl group, a cyclopentyl group, a 1-methylcyclohexyl group, a 1-methylcyclopropyl group, and the like. R¹¹ and R^{11′} each represent, more preferably, an alkyl group having 1 to 8 carbon atoms, and among them, a methyl group, a t-butyl group, a t-amyl group, and a 1-methylcyclohexyl group are even more preferred, a methyl group and a t-butyl group being most preferred.

R¹² and R^{12′} are preferably an alkyl group having 1 to 20 carbon atoms; and examples thereof include, specifically, a methyl group, an ethyl group, a propyl group, a butyl group, an isopropyl group, a t-butyl group, a t-amyl group, a cyclohexyl group, a 1-methylcyclohexyl group, a benzyl group, a methoxymethyl group, a methoxyethyl group, and the like. More preferred are a methyl group, an ethyl group, a propyl group, an isopropyl group, and a t-butyl group, and particularly preferred are a methyl group and an ethyl group.

X¹ and X^{1′} are preferably a hydrogen atom, a halogen atom, or an alkyl group, and more preferably a hydrogen atom.

L is preferably a —CHR¹³— group.

R¹³ is preferably a hydrogen atom or an alkyl group having 1 to 15 carbon atoms. The alkyl group is preferably a chain or cyclic alkyl group. And, groups which have a C═C bond in these alkyl groups are also preferably used. Preferable examples of the alkyl group include a methyl group, an ethyl group, a propyl group, an isopropyl group, a 2,4,4-trimethylpentyl group, a cyclohexyl group, a 2,4-dimethyl-3-cyclohexenyl group, a 3,5-dimetyl-3-cyclohexenyl group, and the like. Particularly preferable R¹³ is a hydrogen atom, a methyl group, an ethyl group, a propyl group, an isopropyl group, or a 2,4-dimethyl-3-cyclohexenyl group.

In the case where R¹¹ and R^{11′} are a tertiary alkyl group and R¹² and R^{12′} are a methyl group, R¹³ is preferably a primary or secondary alkyl group having 1 to 8 carbon atoms (a methyl group, an ethyl group, a propyl group, an isopropyl group, a 2,4-dimethyl-3-cyclohexenyl group, or the like).

In the case where R¹¹ and R^{11′} are a tertiary alkyl group and R¹² and R^{12′} are an alkyl group other than a methyl group, R¹³ is preferably a hydrogen atom.

In the case where R¹¹ and R^{11′} are not a tertiary alkyl group, R¹³ is preferably a hydrogen atom or a secondary alkyl group, and particularly preferably a secondary alkyl group. As the secondary alkyl group for R¹³, an isopropyl group, a 2,4-dimethyl-3-cyclohexenyl group, and a cyclohexyl group are preferred.

The reducing agent described above shows different thermal development performance, color tone of developed silver images, or the like depending on the combinations of R¹¹, R^{11′}, R¹², R^{12′}, and R¹³. Since the performance can be controlled by using two or more reducing agents in combination, it is preferred to use two or more reducing agents in combination depending on the purpose.

Specific examples of the reducing agent according to the invention including the compounds represented by formula (R) are shown below, but the invention is not restricted to these examples.

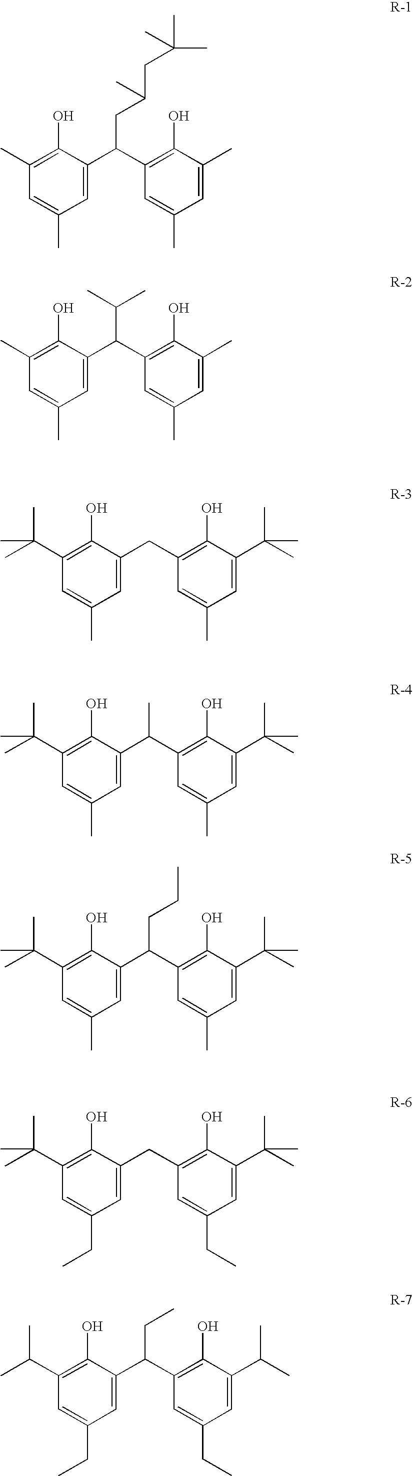

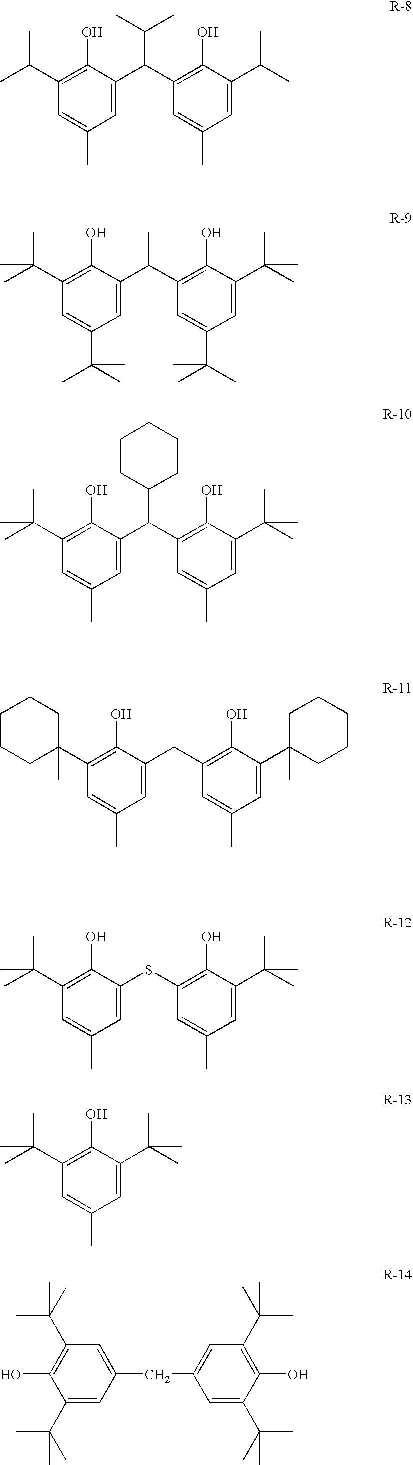

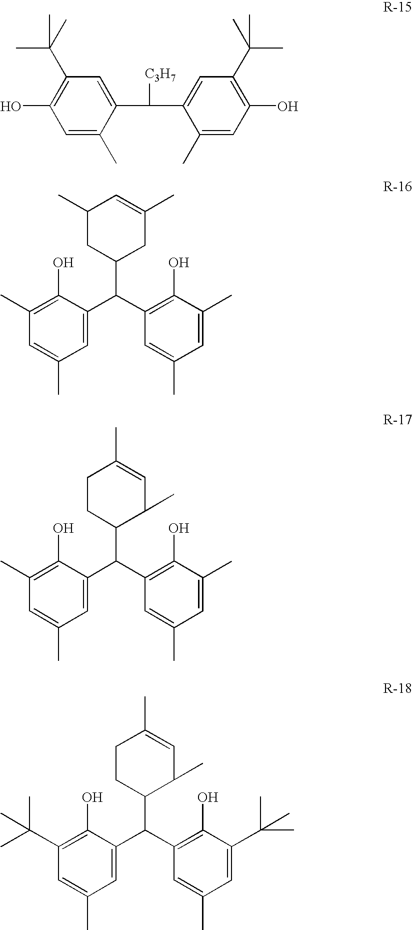

As preferred examples of the reducing agent according to the invention other than those above, there are mentioned compounds described in JP-A Nos. 2001-188314, 2001-209145, 2001-350235, and 2002-156727, and EP No. 1278101A2.

In the present invention, the addition amount of the reducing agent is preferably from 0.1 g/m² to 3.0 g/m², more preferably from 0.2 g/m² to 2.0 g/m², and even more preferably from 0.3 g/m² to 1.0 g/m². It is preferably contained in a range of from 5 mol % to 50 mol %, more preferably from 8 mol % to 30 mol %, and even more preferably from 10 mol % to 20 mol %, with respect to 1 mol of silver on the side having the image forming layer. The reducing agent is preferably contained in the image forming layer.

The reducing agent may be incorporated into the photothermographic material by being contained into the coating solution by any method, such as in the form of a solution, an emulsified dispersion, a solid fine particle dispersion, or the like.

As a well-known emulsified dispersion method, there is mentioned a method comprising dissolving the reducing agent in an oil such as dibutyl phthalate, tricresyl phosphate, dioctyl sebacate, tri(2-ethylhexyl)phosphate, or the like, and an auxiliary solvent such as ethyl acetate, cyclohexanone, or the like, and then adding a surfactant such as sodium dodecylbenzenesulfonate, sodium oleoil-N-methyltaurinate, di(2-ethylhexyl)sodium sulfosuccinate or the like; from which an emulsified dispersion is mechanically produced. During the process, for the purpose of controlling viscosity of oil droplet and refractive index, the addition of polymer such as α-methylstyrene oligomer, poly(t-butylacrylamide), or the like is preferable.

As a solid fine particle dispersion method, there is mentioned a method comprising dispersing the powder of the reducing agent in a proper solvent such as water or the like, by means of ball mill, colloid mill, vibrating ball mill, sand mill, jet mill, roller mill, or ultrasonics, thereby obtaining a solid dispersion. In this process, there may be used a protective colloid (such as poly(vinyl alcohol)), or a surfactant (for instance, an anionic surfactant such as sodium triisopropylnaphthalenesulfonate (a mixture of compounds having the three isopropyl groups in different substitution sites)). In the mills enumerated above, generally used as the dispersion media are beads made of zirconia or the like, and Zr or the like eluting from the beads may be incorporated in the dispersion. Although depending on the dispersing conditions, the amount of Zr or the like incorporated in the dispersion is generally in a range of from 1 ppm to 1000 ppm. It is practically acceptable so long as Zr is incorporated in the photothermographic material in an amount of 0.5 mg or less per 1 g of silver.

Preferably, an antiseptic (for instance, benzisothiazolinone sodium salt) is added in an aqueous dispersion.

The reducing agent is particularly preferably used as a solid particle dispersion, and is added in the form of fine particles having a mean particle size of from 0.01 μm to 10 μm, preferably from 0.05 μm to 5 μm, and more preferably from 0.1 μm to 2 μm. In the application, other solid dispersions are preferably used to be dispersed with this particle size range.

(Nucleator)

The nucleator which can be used in the invention is explained.

Preferable examples of the nucleator include a hydrazine derivative compound represented by the following formula (H), a vinyl compound represented by the following formula (G), and a quaternary onium compound represented by the following formula (P), a cyclic olefin compound represented by formula (A), (B), or (C), and the like.

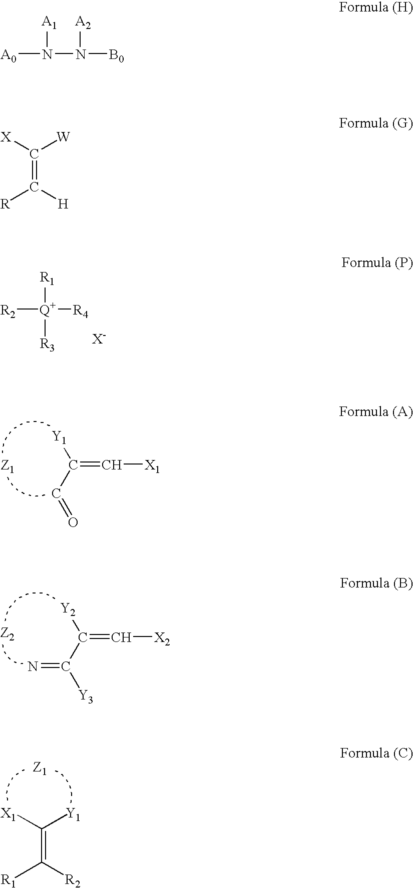

In formula (H), A₀ represents an aliphatic group, an aromatic group, a heterocyclic group, or a -G₀-D₀ group, each of which may have a substituent. B₀ represents a blocking group. Both of A₁ and A₂ represent a hydrogen atom, or one of A₁ or A₂ represents a hydrogen atom and the other represents an acyl group, a sulfonyl group, or an oxalyl group. Herein, G₀ represents one selected from a —CO— group, a —COCO— group, a —CS— group, a —C(═NG₁D₁) group, an —SO— group, an —SO₂— group, or a —P(O)(G₁D₁)- group. G₁ represents a bond, an —O— group, an —S— group, or an —N(D₁)- group; and D₁ represents an aliphatic group, an aromatic group, a heterocyclic group, or a hydrogen atom. In the case where plural D₁s exist in the molecule, they may be identical or different from each other. D₀ represents one selected from a hydrogen atom, an aliphatic group, an aromatic group, a heterocyclic group, an amino group, an alkoxy group, an aryloxy group, an alkylthio group, or an arylthio group. As preferable D₀, a hydrogen atom, an alkyl group, an alkoxy group, an amino group, and the like are described.

In formula (H), the aliphatic group represented by A₀ preferably has 1 to 30 carbon atoms, and is particularly preferably a straight-chain, branched, or cyclic alkyl group having 1 to 20 carbon atoms. Examples thereof include a methyl group, an ethyl group, a t-butyl group, an octyl group, a cyclohexyl group, and a benzyl group. These may be further substituted by a suitable substituent (e.g., an aryl group, an alkoxy group, an aryloxy group, an alkylthio group, an arylthio group, a sulfoxy group, a sulfonamido group, a sulfamoyl group, an acylamino group, a ureido group, or the like).

In formula (H), the aromatic group represented by A₀ is preferably an aryl group of a single or condensed ring. For example, a benzene ring or a naphthalene ring is described. As the heterocyclic group represented by A₀, a heterocycle of a monocycle or condensed ring containing at least one heteroatom selected from among nitrogen, sulfur, and oxygen is preferable; and examples thereof include a pyrrolidine ring, an imidazole ring, a tetrahydrofuran ring, a morpholine ring, a pyridine ring, a pyrimidine ring, a quinoline ring, a thiazole ring, a benzothiazole ring, a thiophene ring, and a furan ring. The aromatic group, heterocyclic group, or -G₀-D₀ group for A₀ may have a substituent. As A₀, particularly preferable are an aryl group and a -G₀-D₀ group.

Further, in formula (H), A₀ preferably contains at least one of a nondiffusing group or an adsorptive group to silver halide. As the nondiffusing group, a ballast group usually used in a non-moving photographic additive such as a coupler is preferable. As the ballast group, a photochemically inactive alkyl group, alkenyl group, alkynyl group, alkoxy group, phenyl group, phenoxy group, alkylphenoxy group, and the like are described, and it is preferred that the substituent portion has 8 or more carbon atoms in total.

In formula (H), examples of the adsorption promoting group to silver halide include thiourea, a thiourethane group, a mercapto group, a thioether group, a thione group, a heterocyclic group, a thioamido heterocyclic group, a mercapto heterocyclic group, an adsorptive group described in JP-A No. 64-90439, and the like.

In formula (H), B₀ represents a blocking group, and is preferably a -G₀-D₀ group. G₀ represents one selected from a —CO— group, a —COCO— group, a —CS— group, a —C(═NG₁D₁) group, an —SO— group, an —SO₂— group, or a —P(O)(G₁D₁)- group. G₀ is preferably a —CO— group or a —COCO— group. G₁ represents a bond, an —O— group, an —S— group, or an —N(D₁)- group; and D₁ represents an aliphatic group, an aromatic group, a heterocyclic group, or a hydrogen atom. In the case where plural D₁s exist in the molecule, they may be identical or different from each other. D₀ represents one selected from a hydrogen atom, an aliphatic group, an aromatic group, a heterocyclic group, an amino group, an alkoxy group, an aryloxy group, an alkylthio group, or an arylthio group. D₀ is preferably a hydrogen atom, an alkyl group, an alkoxy group, or an amino group. Both of A₁ and A₂ represent a hydrogen atom, or one of A₁ or A₂ represents a hydrogen atom and the other represents an acyl group (an acetyl group, a trifluoroacetyl group, a benzoyl group, or the like), a sulfonyl group (a methanesulfonyl group, a toluenesulfonyl group, or the like), or an oxalyl group (an ethoxalyl group or the like).

As specific examples of the compound represented by formula (H), the compounds H-1 to H-35 of chemical formula Nos. 12 to 18 and the compounds H-1-1 to H-4-5 of chemical formula Nos. 20 to 26 in JP-A No. 2002-131864 are described, however the invention is not limited to these examples.

The compounds represented by formula (H) can be easily synthesized by known methods. For example, these can be synthesized by referring to U.S. Pat. Nos. 5,464,738 and 5,496,695.

In addition, hydrazine derivatives preferably used are the compounds H-1 to H-29 described in columns 11 to 20 of U.S. Pat. No. 5,545,505, and the compounds 1 to 12 described in columns 9 to 11 of U.S. Pat. No. 5,464,738. These hydrazine derivatives can be synthesized by known methods.

Next, formula (G) is explained. In formula (G), although X and R are displayed in a cis form, a trans form for X and R is also included in formula (G). This is also similar to the structure display of specific compounds.

In formula (G), X represents an electron-attracting group; and W represents one selected from a hydrogen atom, an alkyl group, an alkenyl group, an alkynyl group, an aryl group, a heterocyclic group, a halogen atom, an acyl group, a thioacyl group, an oxalyl group, an oxyoxalyl group, a thiooxalyl group, an oxamoyl group, an oxycarbonyl group, a thiocarbonyl group, a carbamoyl group, a thiocarbamoyl group, a sulfonyl group, a sulfinyl group, an oxysulfonyl group, a thiosulfonyl group, a sulfamoyl group, an oxysulfinyl group, a thiosulfinyl group, a sulfinamoyl group, a phosphoryl group, a nitro group, an imino group, a N-carbonyl imino group, a N-sulfonyl imino group, a dicyanoethylene group, an ammonium group, a sulfonium group, a phosphonium group, a pyrylium group, or an immonium group.

R represents one selected from a halogen atom, a hydroxy group, an alkoxy group, an aryloxy group, a heterocyclic oxy group, an alkenyloxy group, an acyloxy group, an alkoxycarbonyloxy group, an aminocarbonyloxy group, a mercapto group, an alkylthio group, an arylthio group, a heterocyclic thio group, an alkenylthio group, an acylthio group, an alkoxycarbonylthio group, an aminocarbonylthio group, an organic or inorganic salt of hydroxy group or mercapto group (e.g., a sodium salt, a potassium salt, a silver salt, or the like), an amino group, an alkylamino group, a cyclic amino group (e.g., a pyrrolidino group), an acylamino group, an oxycarbonylamino group, a heterocyclic group (a 5 or 6-membered nitrogen-containing heterocycle, e.g., a benztriazolyl group, an imidazolyl group, a triazolyl group, a tetrazolyl group, or the like), a ureido group, or a sulfonamido group. X and W, and X and R may bond to each other to form a cyclic structure. Examples of the ring formed by X and W include pyrazolone, pyrazolidinone, cyclopentanedione, β-ketolactone, β-ketolactam, and the like.

Explaining about formula (G) further, the electron-attracting group represented by X is a substituent whose substituent constant σp yields a positive value. Specifically, a substituted alkyl group (halogen-substituted alkyl or the like), a substituted alkenyl group (cyanovinyl or the like), a substituted or unsubstituted alkynyl group (trifluoromethylacetylenyl, cyanoacetylenyl, or the like), a substituted aryl group (cyanophenyl or the like), a substituted or unsubstituted heterocyclic group (pyridyl, triazinyl, benzoxazolyl, or the like), a halogen atom, a cyano group, an acyl group (acetyl, trifluoroacetyl, formyl, or the like), a thioacetyl group (thioacetyl, thioformyl, or the like), an oxalyl group (methyloxalyl or the like), an oxyoxalyl group (ethoxalyl or the like), a thiooxalyl group (ethylthiooxalyl or the like), an oxamoyl group (methyloxamoyl or the like), an oxycarbonyl group (ethoxycarbonyl or the like), a carboxy group, a thiocarbonyl group (ethylthiocarbonyl or the like), a carbamoyl group, a thiocarbamoyl group, a sulfonyl group, a sulfinyl group, an oxysulfonyl group (ethoxysulfonyl or the like), a thiosulfonyl group (ethylthiosulfonyl or the like), a sulfamoyl group, an oxysulfinyl group (methoxysulfinyl or the like), a thiosulfinyl group (methylthiosulfinyl or the like), a sulfinamoyl group, a phosphoryl group, a nitro group, an imino group, an N-carbonylimino group (N-acetylimino or the like), an N-sulfonylimino group (N-methanesulfonylimino or the like), a dicyanoethylene group, an ammonium group, a sulfonium group, a phosphonium group, a pyrylium group, an immonium group, and the like are described, and a heterocyclic one formed by an ammonium group, sulfonium group, phosphonium group, immonium group, or the like is also included. The substituent having σp value of 0.30 or higher is particularly preferable.

Examples of the alkyl group represented by W include methyl, ethyl, trifluoromethyl, and the like; examples of the alkenyl group for W include vinyl, halogen-substituted vinyl, cyanovinyl, and the like; examples of the alkynyl group for W include acetylenyl, cyanoacetylenyl, and the like; examples of the aryl group for W include nitrophenyl, cyanophenyl, pentafluorophenyl, and the like; and examples of the heterocyclic group for W include pyridyl, pyrimidyl, triazinyl, succinimide, tetrazolyl, triazolyl, imidazolyl, benzoxazolyl, and the like. The electron-attracting group having a positive σp value is preferable for W, and the one having σp value of 0.30 or higher is more preferable.

Among the substituents of R described above, a hydroxy group, a mercapto group, an alkoxy group, an alkylthio group, a halogen atom, an organic or inorganic salt of hydroxy group or mercapto group, and a heterocyclic group are preferably described. More preferably, a hydroxy group, an alkoxy group, an organic or inorganic salt of hydroxy group or mercapto group, and a heterocyclic group are described, and particularly preferably, a hydroxy group and an organic or inorganic salt of hydroxy group or mercapto group are described.

And among the substituents of X and W described above, those having a thioether bond in the substituent are preferable.

As specific examples of the compound represented by formula (G), the compounds 1-1 to 92-7 of chemical formula Nos. 27 to 50 described in JP-A No. 2002-131864 are described, however the invention is not limited to these examples.

In formula (P), Q represents a nitrogen atom or a phosphorus atom. R₁, R₂, R₃, and R₄ each independently represent a hydrogen atom or a substituent, and X⁻ represents an anion. In addition, R₁ to R₄ may bond to each other to form a cyclic structure.

As the substituent represented by R₁ to R₄, an alkyl group (a methyl group, an ethyl group, a propyl group, a butyl group, a hexyl group, a cyclohexyl group, or the like), an alkenyl group (an allyl group, a butenyl group, or the like), an alkynyl group (a propargyl group, a butynyl group, or the like), an aryl group (a phenyl group, a naphthyl group, or the like), a heterocyclic group (a piperidinyl group, a piperazinyl group, a morpholinyl group, a pyridyl group, a furyl group, a thienyl group, a tetrahydrofuryl group, a tetrahydrothienyl group, a sulforanyl group, or the like), an amino group, and the like are described.

As the ring formed by linking R₁ to R₄ each other, a piperidine ring, a morpholine ring, a piperazine ring, a quinuclidine ring, a pyridine ring, a pyrrole ring, an imidazole ring, a triazole ring, a tetrazole ring, and the like are described.

The group represented by R₁ to R₄ may have a substituent such as a hydroxy group, an alkoxy group, an aryloxy group, a carboxy group, a sulfo group, an alkyl group, an aryl group, or the like. R¹, R₂, R₃, and R₄ are each preferably a hydrogen atom or an alkyl group.

As the anion represented by X⁻, an inorganic and organic anion such as a halogen ion, a sulfate ion, a nitrate ion, an acetate ion, a p-toluenesulfonate ion, and the like are described.

As a structure of formula (P), the structure described in paragraph Nos. 0153 to 0163 in JP-A No. 2002-131864 is even more preferable.

As specific compounds of formula (P), the compounds P-1 to P-52 and T-1 to T-18 of chemical formula Nos. 53 to 62 in JP-A No. 2002-131864 can be described, however the invention is not limited to these compounds.

The quaternary onium compound described above can be synthesized by referring to known methods. For example, the tetrazolium compound described above can be synthesized by referring to the method described in Chemical Reviews, vol. 55, pages 335 to 483.

Next, the compounds represented by formulae (A) or (B) are explained in detail. In formula (A), Z₁ represents a nonmetallic atomic group which forms a 5 to 7-membered cyclic structure with —Y₁—C(═CH—X₁)—C(═O)—. Z₁ is preferably an atomic group selected from among carbon, oxygen, sulfur, nitrogen, and hydrogen; and several atoms selected from these link together by single bond or double bond to form a 5 to 7-membered cyclic structure with —Y₁—C(═CH—X₁)—C(═O)—. Z₁ may have a substituent, and Z₁ itself may a part of an aromatic or non-aromatic carbon ring, or an aromatic or non-aromatic heterocycle, and in this case, a 5 to 7-membered cyclic structure formed by Z, with —Y₁—C(═CH—X₁)—C(═O)— forms a condensed cyclic structure.

In formula (B), Z₂ represents a nonmetallic atomic group which forms a 5 to 7-membered cyclic structure with —Y₂—C(═CH—X₂)—C(Y₃)═N—. Z₂ is preferably an atomic group selected from among carbon, oxygen, sulfur, nitrogen, and hydrogen; and several atoms selected from these link together by single bond or double bond to form a 5 to 7-membered cyclic structure with —Y₂—C(═CH—X₂)—C(Y₃)═N—. Z₂ may have a substituent, and Z₂ itself may be a part of an aromatic or non-aromatic carbon ring, or an aromatic or non-aromatic heterocycle, and in this case, a 5 to 7-membered cyclic structure formed by Z₂ with —Y₂—C(═CH—X₂)—C(Y₃)═N— forms a condensed cyclic structure.

In the case where Z₁ or Z₂ has a substituent, the substituent is selected from those listed below. Namely, typical examples of the substituent include a halogen atom (a fluorine atom, chlorine atom, bromine atom, or iodine atom), an alkyl group (including an aralkyl group, a cycloalkyl group, an active methine group, and the like), an alkenyl group, an alkynyl group, an aryl group, a heterocyclic group, a heterocyclic group containing a quaternary nitrogen atom (e.g., a pyridinio group), an acyl group, an alkoxycarbonyl group, an aryloxycarbonyl group, a carbamoyl group, a carboxy group or a salt thereof, a sulfonylcarbamoyl group, an acylcarbamoyl group, a sulfamoylcarbamoyl group, a carbazoyl group, an oxalyl group, an oxamoyl group, a cyano group, a thiocarbamoyl group, a hydroxy group, an alkoxy group (including a group in which ethylene oxy group units or propylene oxy group units are repeated), an aryloxy group, a heterocyclic oxy group, an acyloxy group, an alkoxy carbonyloxy group, an aryloxy carbonyloxy group, a carbamoyloxy group, a sulfonyloxy group, an amino group, an alkylamino group, an arylamino group, a heterocyclic amino group, an N-substituted nitrogen-containing heterocyclic group, an acylamino group, a sulfonamido group, a ureido group, a thioureido group, an imido group, an alkoxycarbonylamino group, an aryloxycarbonylamino group, a sulfamoylamino group, a semicarbazide group, a thiosemicarbazide group, a hydrazino group, a quaternary ammonio group, an oxamoylamino group, an alkylsulfonylureido group, an arylsulfonylureido group, an acylureido group, an acylsulfamoylamino group, a nitro group, a mercapto group, an alkylthio group, an arylthio group, a heterocyclic thio group, an alkylsulfonyl group, an arylsulfonyl group, an alkylsulfinyl group, an arylsulfinyl group, a sulfo group or a salt thereof, a sulfamoyl group, an acylsulfamoyl group, a sulfonylsulfamoyl group or a salt thereof, a group containing amido phosphate or phosphoric acid ester structure, a silyl group, a stannyl group, and the like. These substituents may be further substituted by these substituents.

Next, Y₃ is explained. In formula (B), Y₃ represents a hydrogen atom or a substituent, and when Y₃ represents a substituent, the following groups are specifically described as the substituent. Namely, an alkyl group, an aryl group, a heterocyclic group, a cyano group, an acyl group, an alkoxycarbonyl group, an aryloxycarbonyl group, a carbamoyl group, an amino group, an alkylamino group, an arylamino group, a heterocyclic amino group, an acylamino group, a sulfonamido group, a ureido group, a thioureido group, an imido group, an alkoxy group, an aryloxy group, an alkylthio group, an arylthio group, a heterocyclic thio group, and the like are described. These substituents may be substituted by any substituents, and specifically, examples of the substituent which Z₁ or Z₂ may have are described.

In formulae (A) and (B), X₁ and X₂ each independently represent one selected from a hydroxy group (or a salt thereof), an alkoxy group (e.g., a methoxy group, an ethoxy group, a propoxy group, an isopropoxy group, an octyloxy group, a dodecyloxy group, a cetyloxy group, a t-butoxy group, or the like), an aryloxy group (e.g., a phenoxy group, a p-t-pentylphenoxy group, a p-t-octylphenoxy group, or the like), a heterocyclic oxy group (e.g., a benzotriazolyl-5-oxy group, a pyridinyl-3-oxy group, or the like), a mercapto group (or a salt thereof), an alkylthio group (e.g., methylthio group, an ethylthio group, a butylthio group, a dodecylthio group, or the like), an arylthio group (e.g., a phenylthio group, a p-dodecylphenylthio group, or the like), a heterocyclic thio group (e.g., a 1-phenyltetrazoyl-5-thio group, a 2-methyl-1-phenyltriazolyl-5-thio group, a mercaptothiadiazolylthio group, or the like), an amino group, an alkylamino group (e.g., a methylamino group, a propylamino group, an octylamino group, a dimethylamino group, or the like), an arylamino group (e.g., an anilino group, a naphthylamino group, an o-methoxyanilino group, or the like), a heterocyclic amino group (e.g., a pyridylamino group, a benzotriazole-5-ylamino group, or the like), an acylamino group (e.g., an acetamido group, an octanoylamino group, a benzoylamino group, or the like), a sulfonamido group (e.g., a methanesulfonamido group, a benzenesulfonamido group a dodecylsulfonamido group, or the like), or a heterocyclic group.

Herein, a heterocyclic group is an aromatic or non-aromatic, saturated or unsaturated, monocyclic or condensed-ring, substituted or unsubstituted heterocyclic group. Examples thereof include an N-methylhydantoyl group, an N-phenylhydantoyl group, a succinimido group, a phthalimido group, an N,N′-dimethylurazolyl group, an imidazolyl group, a benzotriazolyl group, an indazolyl group, a morpholino group, a 4,4-dimethyl-2,5-dioxo-oxazolyl group, and the like.

And herein, a salt represents a salt of an alkali metal (sodium, potassium, or lithium), a salt of an alkali earth metal (magnesium or calcium), a silver salt, a quaternary ammonium salt (a tetraethylammonium salt, a dimethylcetylbenzylammonium salt, or the like), a quaternary phosphonium salt, or the like. In formulae (A) and (B), Y, and Y₂ each represent —C(═O)— or —SO₂—.

Preferable ranges of the compounds represented by formula (A) or (B) are described in JP-A No. 11-231459, paragraph Nos. 0027 to 0043. As specific examples of the compound represented by formula (A) or (B), the compounds 1 to 110 of Table 1 to Table 8 in JP-A No. 11-231459 are described, however the invention is not limited to these.

Next, the compound represented by formula (C) according to the present invention is explained in detail. In formula (C), X₁ represents an oxygen atom, a sulfur atom, or a nitrogen atom. In the case where X₁ is a nitrogen atom, the bond of X₁ and Z₁ may be either a single bond or a double bond, and in the case of a single bond, the nitrogen atom may have a hydrogen atom or any substituent. Examples of the substituent include an alkyl group (including an aralkyl group, a cycloalkyl group, an active methine group, and the like), an alkenyl group, an alkynyl group, an aryl group, a heterocyclic group, an acyl group, an alkoxycarbonyl group, an aryloxycarbonyl group, a carbamoyl group, an alkylsulfonyl group, an arylsulfonyl group, a heterocyclic sulfonyl group, and the like.

Y₁ represents a group represented by —C(═O)—, —C(═S)—, —SO—, —SO₂—, —C(═NR₃)—, or —(R₄)C═N—. Z₁ represents a nonmetallic atomic group which forms a 5 to 7-membered ring including X₁ and Y₁. The atomic group to form the ring is an atomic group which consists of 2 to 4 atoms that are other than metal atoms, and these atoms may be combined by single bond or double bond, and these may have a hydrogen atom or any substituent (e.g., an alkyl group, an aryl group, a heterocyclic group, an alkoxy group, an alkylthio group, an acyl group, an amino group, or an alkenyl group). When Z₁ forms a 5 to 7-membered ring including X₁ and Y₁, the ring is a saturated or unsaturated heterocycle, and may be monocyclic or may have a condensed ring. When Y₁ is the group represented by C(═NR₃) or (R₄)C═N, the condensed ring of this case may be formed by bonding R₃ or R₄ with the substituent of Z₁.

In formula (C), R₁, R₂, R₃, and R₄ each independently represent a hydrogen atom or a substituent. However, R₁ and R₂ never bond to each other to form a cyclic structure.

When R₁ and R₂ represent a monovalent substituent, the following groups are described as the monovalent substituent.

For example, a halogen atom (a fluorine atom, chlorine atom, bromine atom, or iodine atom), an alkyl group (including an aralkyl group, a cycloalkyl group, an active methine group, and the like), an alkenyl group, an alkynyl group, an aryl group, a heterocyclic group, a heterocyclic group containing a quaternary nitrogen atom (e.g., a pyridinio group), an acyl group, an alkoxycarbonyl group, an aryloxycarbonyl group, a carbamoyl group, a carboxy group or a salt thereof, a sulfonylcarbamoyl group, an acylcarbamoyl group, a sulfamoylcarbamoyl group, a carbazoyl group, an oxalyl group, an oxamoyl group, a cyano group, a thiocarbamoyl group, a hydroxy group or a salt thereof, an alkoxy group (including a group in which ethylene oxy group units or propylene oxy group units are repeated), an aryloxy group, a heterocyclic oxy group, an acyloxy group, an alkoxycarbonyloxy group, an aryloxycarbonyloxy group, a carbamoyloxy group, a sulfonyloxy group, an amino group, an alkylamino group, an arylamino group, an heterocyclic amino group, an N-substituted nitrogen-containing heterocyclic group, an acylamino group, a sulfonamido group, a ureido group, a thioureido group, an imido group, an alkoxycarbonylamino group, an aryloxycarbonylamino group, a sulfamoylamino group, a semicarbazide group, a thiosemicarbazide group, a hydrazino group, a quaternary ammonio group, an oxamoylamino group, an alkylsulfonylureido group, an arylsulfonylureido group, an acylureido group, an acylsulfamoylamino group, a nitro group, a mercapto group or a salt thereof, an alkylthio group, an arylthio group, an heterocyclic thio group, an alkylsulfonyl group, an arylsulfonyl group, an alkylsulfinyl group, an arylsulfinyl group, a sulfo group or a salt thereof, a sulfamoyl group, an acylsulfamoyl group, a sulfonylsulfamoyl group or a salt thereof, a phosphoryl group, a group containing amido phosphate or phosphoric acid ester structure, a silyl group, a stannyl group, and the like are described. These substituents may be further substituted by these monovalent substituents.

When R₃ or R₄ represents a substituent, the same substituents as what R₁ and R₂ may have except the halogen atom can be described as the substituent. R₃ or R₄ may further link to Z₁ to form a condensed ring.

Next, among the compounds represented by formula (C), preferable compounds are explained. In formula (C), Z₁ is preferably an atomic group which forms a 5 to 7-membered ring with X₁ and Y₁, and is preferably an atomic group which consists of 2 to 4 atoms selected from among carbon, nitrogen, sulfur, and oxygen. The heterocycle, which is formed by Z₁ with X₁ and Y₁, preferably has 3 to 40 carbon atoms in total, more preferably 3 to 25 carbon atoms in total, and most preferably 3 to 20 carbon atoms in total. Z₁ preferably comprises at least one carbon atom.

In formula (C), Y₁ is preferably —C(═O)—, —C(═S)—, —SO₂—, or —(R₄)C═N—, particularly preferably —C(═O)—, —C(═S)—, or —SO₂—, and most preferably —C(═O)—.

In formula (C), in the case where R₁ or R₂ represents a monovalent substituent, the monovalent substituent represented by R₁ or R₂ is preferably one of the following groups having 0 to 25 carbon atoms in total, namely, those are an alkyl group, an aryl group, a heterocyclic group, an alkoxy group, an aryloxy group, a heterocyclic oxy group, an alkylthio group, an arylthio group, a heterocyclic thio group, an amino group, an alkylamino group, an arylamino group, a heterocyclic amino group, a ureido group, an imido group, an acylamino group, a hydroxy group or a salt thereof, a mercapto group or a salt thereof, and an electron-attracting substituent. Herein, an electron-attracting substituent means a substituent whose Hammett substituent constant σp yields a positive value, and specific examples thereof include a cyano group, a sulfamoyl group, an alkylsulfonyl group, an arylsulfonyl group, a sulfonamido group, an imino group, a nitro group, a halogen atom, an acyl group, a formyl group, a phosphoryl group, a carboxy group (or a salt thereof), a sulfo group (or a salt thereof), a saturated or unsaturated heterocyclic group, an alkenyl group, an alkynyl group, an acyloxy group, an acylthio group, a sulfonyloxy group, and an aryl group substituted by the above-described electron-attracting group. These groups may have any substituents.

In formula (C), when R₁ or R₂ represents a monovalent substituent, more preferable are an alkoxy group, an aryloxy group, a heterocyclic oxy group, an alkylthio group, an arylthio group, a heterocyclic thio group, an amino group, an alkylamino group, an arylamino group, a heterocyclic amino group, a ureido group, an imido group, an acylamino group, a sulfonamido group, a heterocyclic group, a hydroxy group or a salt thereof, a mercapto group or a salt thereof, and the like. In formula (C), R₁ and R₂ are particularly preferably a hydrogen atom, an alkoxy group, an aryloxy group, an alkylthio group, an arylthio group, a heterocyclic group, a hydroxy group or a salt thereof, a mercapto group or a salt thereof, or the like. In formula (C), most preferably, one of R₁ or R₂ is a hydrogen atom and the other is an alkoxy group, an aryloxy group, an alkylthio group, an arylthio group, a heterocyclic group, a hydroxy group or a salt thereof, or a mercapto group or a salt thereof.

In formula (C), when R₃ represents a substituent, R₃ is preferably an alkyl group having 1 to 25 carbon atoms in total (including an aralkyl group, a cycloalkyl group, an active methine group, and the like), an alkenyl group, aryl group, a heterocyclic group, a heterocyclic group containing a quaternary nitrogen atom (e.g., a pyridinio group), an acyl group, an alkoxycarbonyl group, an aryloxycarbonyl group, a carbamoyl group, an alkylsulfonyl group, an arylsulfonyl group, an alkylsulfinyl group, an arylsulfinyl group, a sulfosulfamoyl group, an alkoxy group, an aryloxy group, a heterocyclic oxy group, an alkylthio group, an arylthio group, a heterocyclic thio group, an amino group, or the like. An alkyl group and an aryl group are particularly preferable.

In formula (C), when R₄ represents a substituent, R₄ is preferably an alkyl group having 1 to 25 carbon atoms in total (including an aralkyl group, a cycloalkyl group, an active methine group, and the like), an aryl group, a heterocyclic group, a heterocyclic group containing a quaternary nitrogen atom (e.g., a pyridinio group), an acyl group, an alkoxycarbonyl group, an aryloxycarbonyl group, a carbamoyl group, an alkylsulfonyl group, an arylsulfonyl group, an alkylsulfinyl group, an arylsulfinyl group, a sulfosulfamoyl group, an alkoxy group, an aryloxy group, a heterocyclic oxy group, an alkylthio group, an arylthio group, a heterocyclic thio group, or the like. Particularly preferably, an alkyl group, an aryl group, an alkoxy group, an aryloxy group, a heterocyclic oxy group, an alkylthio group, an arylthio group, a heterocyclic thio group, and the like are described. When Y₁ represents C(R₄)═N, the carbon atom in Y₁ bonds with the carbon atom substituted by X₁ or Y₁.

Specific compounds of formula (C) are represented by the compounds A-1 to A-230 of chemical formula Nos. 6 to 18 described in JP-A No. 11-133546; however the invention is not limited to these compounds.

The addition amount of the above-described nucleator is in a range of from 10⁻⁵ mol to 1 mol, and preferably from 10⁻⁴ mol to 5×10⁻¹ mol, with respect to 1 mol of organic silver salt.

The nucleator described above may be incorporated into the photothermographic material by being contained into the coating solution by any method, such as in the form of a solution, an emulsified dispersion, a solid fine particle dispersion, or the like.

As a well-known emulsified dispersion method, there is mentioned a method comprising dissolving the nucleator in an oil such as dibutyl phthalate, tricresyl phosphate, dioctyl sebacate, tri(2-ethylhexyl)phosphate, or the like, and an auxiliary solvent such as ethyl acetate, cyclohexanone, or the like, and then adding a surfactant such as sodium dodecylbenzenesulfonate, sodium oleoil-N-methyltaurinate, di(2-ethylhexyl)sodium sulfosuccinate or the like; from which an emulsified dispersion is mechanically produced. During the process, for the purpose of controlling viscosity of oil droplet and refractive index, the addition of polymer such as α-methylstyrene oligomer, poly(t-butylacrylamide), or the like is preferable.

As a solid fine particle dispersion method, there is mentioned a method comprising dispersing the powder of the nucleator in a proper solvent such as water or the like, by means of ball mill, colloid mill, vibrating ball mill, sand mill, jet mill, roller mill, or ultrasonics, thereby obtaining a solid dispersion. In this process, there may also be used a protective colloid (such as poly(vinyl alcohol)), or a surfactant (for instance, an anionic surfactant such as sodium triisopropylnaphthalenesulfonate (a mixture of compounds having the three isopropyl groups in different substitution sites)). In the mills enumerated above, generally used as the dispersion media are beads made of zirconia or the like, and Zr or the like eluting from the beads may be incorporated in the dispersion. Although depending on the dispersing conditions, the amount of Zr or the like incorporated in the dispersion is generally in a range of from 1 ppm to 1000 ppm. It is practically acceptable so long as Zr is incorporated in the photothermographic material in an amount of 0.5 mg or less per 1 g of silver.

Preferably, an antiseptic (for instance, benzisothiazolinone sodium salt) is added in an aqueous dispersion.

The nucleator is particularly preferably used as a solid particle dispersion, and is added in the form of fine particles having a mean particle size of from 0.01 μm to 10 μm, preferably from 0.05 μm to 5 μm and, more preferably from 0.1 μm to 2 μm. In the application, other solid dispersions are preferably used to be dispersed with this particle size range.

In the photothermographic material which is subjected to a rapid development where time period for development is 20 seconds or less, the compound represented by formula (H) or (P) is preferably used, and the compound represented by formula (H) is particularly preferably used, among the nucleators described above.

In the photothermographic material where low fog is required, the compound represented by formula (G), (A), (B), or (C) is preferably used, and the compound represented by formula (A) or (B) is particularly preferably used. Moreover, in the photothermographic materials having a few change in photographic performance against environmental conditions when used under various environmental conditions (temperature and humidity), the compound represented by formula (C) is preferably used.

Although preferred specific compounds among the above-mentioned nucleators are shown below, the invention is not limited to these compounds.

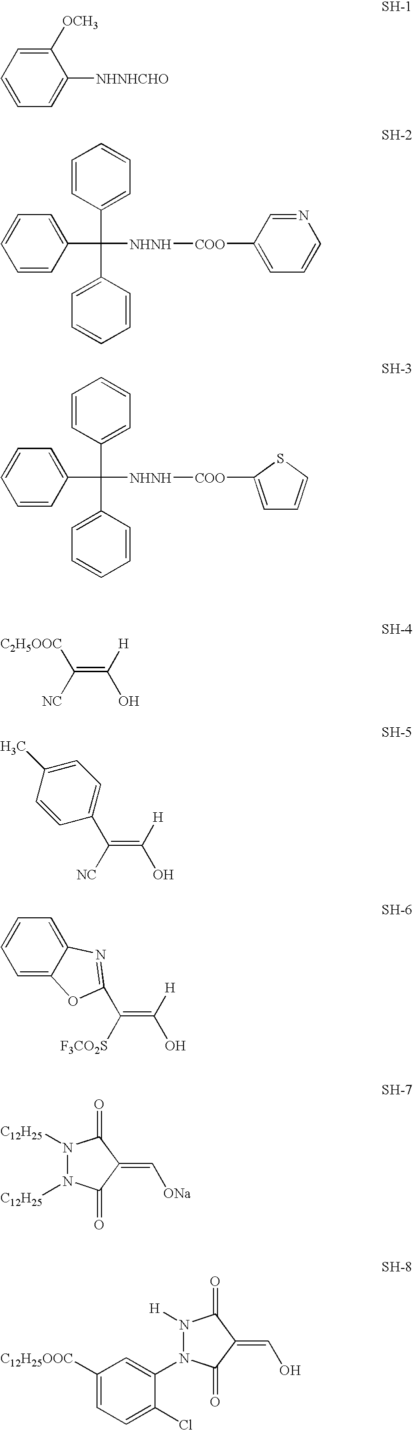

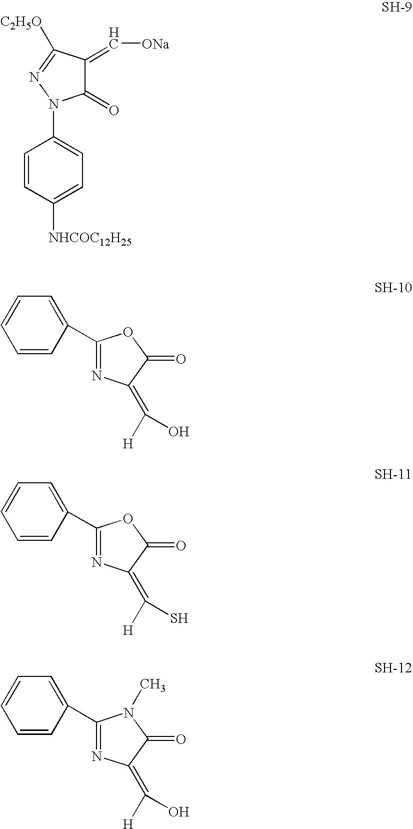

(Solvent for Organic Silver Salt)

In the immunoassay method of the present invention, the photothermographic material preferably includes a solvent (solubilizer) for organic silver salt. As the solvent used for the invention, preferred are phthalazinones(phthalazinone, phthalazinone derivatives, or metal salts thereof; for example, 4-(1-naphthyl)phthalazinone, 6-chlorophthalazinone, 5,7-dimethoxyphthalazinone, and 2,3-dihydro-1,4-phthalazinedione); combinations of phthalazinones and phthalic acids (e.g., phthalic acid, 4-methylphthalic acid, 4-nitrophthalic acid, diammonium phthalate, sodium phthalate, potassium phthalate, and tetrachlorophthalic anhydride); phthalazines(phthalazine, phthalazine derivatives, or metal salts thereof; for example, 4-(1-naphthyl)phthalazine, 6-isopropylphthalazine, 6-tert-butylphthalazine, 6-chlorophthalazine, 5,7-dimethoxyphthalazine, and 2,3-dihydrophthalazine); and combinations of phthalazines and phthalic acids. Particularly preferred are combinations of phthalazines and phthalic acids. Among them, particularly preferable are the combination of 6-isopropylphthalazine and phthalic acid, and the combination of 6-isopropylphthalazine and 4-methylphthalic acid.

The solvent used in the invention is preferably used in a range of from 0.1 mol to 10 mol in terms of molar ratio with respect to the silver ion.

(Development Accelerator)

In the photothermographic material according to the invention, as a development accelerator, sulfonamido phenol compounds described in the specification of JP-A No. 2000-267222, and represented by formula (A) described in the specification of JP-A No. 2000-330234; hindered phenol compounds represented by formula (II) described in JP-A No. 2001-92075; hydrazine compounds described in the specification of JP-A No. 10-62895, represented by formula (I) described in the specification of JP-A No. 11-15116, represented by formula (D) described in the specification of JP-A No. 2002-156727, and represented by formula (1) described in the specification of JP-A No. 2002-278017; and phenol or naphthol compounds represented by formula (2) described in the specification of JP-A No. 2001-264929 are used preferably. Further, phenol compounds described in JP-A Nos. 2002-311533 and 2002-341484 are also preferable. Naphthol compounds described in JP-A No. 2003-66558 are particularly preferable. The development accelerator is used in a range of from 0.1 mol % to 20 mol %, preferably in a range of from 0.5 mol % to 10 mol %, and more preferably in a range of from 1 mol % to 5 mol %, with respect to the reducing agent. The introducing method to the photothermographic material includes similar methods to those for the reducing agent, and it is particularly preferred to add the development accelerator as a solid dispersion or an emulsified dispersion. In the case of adding the development accelerator as an emulsified dispersion, it is preferred to add it as an emulsified dispersion dispersed by using a solvent having a high boiling point which is solid at a normal temperature and an auxiliary solvent having a low boiling point, or to add it as a so-called oilless emulsified dispersion not using a solvent having a high boiling point.

In the present invention, among the development accelerators described above, it is more preferred to use hydrazine compounds described in the specifications of JP-A Nos. 2002-156727 and 2002-278017, and naphthol compounds described in the specification of JP-A No. 2003-66558.

Particularly preferred development accelerators according to the invention are compounds represented by the following formulae (A-1) or (A-2).

Q₁-NHNH-Q₂ Formula (A-1)

In the formula, Q₁ represents an aromatic group or heterocyclic group which bonds to —NHNH-Q₂ at a carbon atom, and Q₂ represents one selected from a carbamoyl group, an acyl group, an alkoxycarbonyl group, an aryloxycarbonyl group, a sulfonyl group, or a sulfamoyl group.

In formula (A-1), the aromatic group or heterocyclic group represented by Q₁ is preferably a 5- to 7-membered unsaturated ring. Preferred examples thereof include a benzene ring, a pyridine ring, a pyrazine ring, a pyrimidine ring, a pyridazine ring, a 1,2,4-triazine ring, a 1,3,5-triazine ring, a pyrrole ring, an imidazole ring, a pyrazole ring, a 1,2,3-triazole ring, a 1,2,4-triazole ring, a tetrazole ring, a 1,3,4-thiadiazole ring, a 1,2,4-thiadiazole ring, a 1,2,5-thiadiazole ring, a 1,3,4-oxadiazole ring, a 1,2,4-oxadiazole ring, a 1,2,5-oxadiazole ring, a thiazole ring, an oxazole ring, an isothiazole ring, an isooxazole ring, a thiophene ring, and the like. Condensed rings in which the rings described above are condensed to each other are also preferred.

The rings described above may have substituents, and in the case where they have two or more substituents, the substituents may be identical or different from each other. Examples of the substituent include a halogen atom, an alkyl group, an aryl group, a carbonamido group, an alkylsulfonamido group, an arylsulfonamido group, an alkoxy group, an aryloxy group, an alkylthio group, an arylthio group, a carbamoyl group, a sulfamoyl group, a cyano group, an alkylsulfonyl group, an arylsulfonyl group, an alkoxycarbonyl group, an aryloxycarbonyl group, and an acyl group. In the case where the substituents are groups capable of substitution, they may further have a substituent, and examples of preferred substituent include a halogen atom, an alkyl group, an aryl group, a carbonamido group, an alkylsulfonamido group, an arylsulfonamido group, an alkoxy group, an aryloxy group, an alkylthio group, an arylthio group, an acyl group, an alkoxycarbonyl group, an aryloxycarbonyl group, a carbamoyl group, a cyano group, a sulfamoyl group, an alkylsulfonyl group, an arylsulfonyl group, and an acyloxy group.

The carbamoyl group represented by Q₂ is a carbamoyl group preferably having 1 to 50 carbon atoms, and more preferably having 6 to 40 carbon atoms; and examples thereof include unsubstituted carbamoyl, methylcarbamoyl, N-ethylcarbamoyl, N-propylcarbamoyl, N-sec-butylcarbamoyl, N-octylcarbamoyl, N-cyclohexylcarbamoyl, N-tert-butylcarbamoyl, N-dodecylcarbamoyl, N-(3-dodecyloxypropyl)carbamoyl, N-octadecylcarbamoyl, N-{3-(2,4-tert-pentylphenoxy)propyl}carbamoyl, N-(2-hexyldecyl)carbamoyl, N-phenylcarbamoyl, N-(4-dodecyloxyphenyl)carbamoyl, N-(2-chloro-5-dodecyloxycarbonylphenyl)carbamoyl, N-naphthylcarbamoyl, N-3-pyridylcarbamoyl, and N-benzylcarbamoyl.

The acyl group represented by Q₂ is an acyl group preferably having 1 to 50 carbon atoms, and more preferably having 6 to 40 carbon atoms; and examples thereof include formyl, acetyl, 2-methylpropanoyl, cyclohexylcarbonyl, octanoyl, 2-hexyldecanoyl, dodecanoyl, chloroacetyl, trifluoroacetyl, benzoyl, 4-dodecyloxybenzoyl, and 2-hydroxymethylbenzoyl. The alkoxycarbonyl group represented by Q₂ is an alkoxycarbonyl group preferably having 2 to 50 carbon atoms, and more preferably having 6 to 40 carbon atoms; and examples thereof include methoxycarbonyl, ethoxycarbonyl, isobutyloxycarbonyl, cyclohexyloxycarbonyl, dodecyloxycarbonyl, and benzyloxycarbonyl.

The aryloxycarbonyl group represented by Q₂ is an aryloxycarbonyl group preferably having 7 to 50 carbon atoms, and more preferably having 7 to 40 carbon atoms; and examples thereof include phenoxycarbonyl, 4-octyloxyphenoxycarbonyl, 2-hydroxymethylphenoxycarbonyl, and 4-dodecyloxyphenoxycarbonyl. The sulfonyl group represented by Q₂ is a sulfonyl group preferably having 1 to 50 carbon atoms, and more preferably having 6 to 40 carbon atoms; and examples thereof include methylsulfonyl, butylsulfonyl, octylsulfonyl, 2-hexadecylsulfonyl, 3-dodecyloxypropylsulfonyl, 2-octyloxy-5-tert-octylphenylsulfonyl, and 4-dodecyloxyphenylsulfonyl.

The sulfamoyl group represented by Q₂ is a sulfamoyl group preferably having 0 to 50 carbon atoms, and more preferably having 6 to 40 carbon atoms; and examples thereof include unsubstituted sulfamoyl, an N-ethylsulfamoyl group, N-(2-ethylhexyl)sulfamoyl, N-decylsulfamoyl, N-hexadecylsulfamoyl, N-{3-(2-ethylhexyloxy)propyl}sulfamoyl, N-(2-chloro-5-dodecyloxycarbonylphenyl)sulfamoyl, and N-(2-tetradecyloxyphenyl)sulfamoyl. The group represented by Q₂ may further have a group mentioned as the example of the substituent of 5- to 7-membered unsaturated ring represented by Q₁ described above at the position capable of substitution. In the case where the group represented by Q₂ has two or more substituents, such substituents may be identical or different from one another.

Next, preferred range for the compound represented by formula (A-1) is to be described. A 5- or 6-membered unsaturated ring is preferred for Q₁, and a benzene ring, a pyrimidine ring, a 1,2,3-triazole ring, a 1,2,4-triazole ring, a tetrazole ring, a 1,3,4-thiadiazole ring, a 1,2,4-thiadiazole ring, a 1,3,4-oxadiazole ring, a 1,2,4-oxadiazole ring, a thiazole ring, an oxazole ring, an isothiazole ring, an isooxazole ring, and a ring in which the ring described above is condensed with a benzene ring or unsaturated heterocycle are more preferred. Further, Q₂ is preferably a carbamoyl group, and particularly preferably a carbamoyl group having a hydrogen atom on the nitrogen atom.

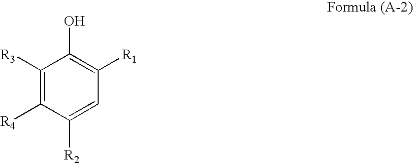

In formula (A-2), R₁ represents one selected from an alkyl group, an acyl group, an acylamino group, a sulfonamido group, an alkoxycarbonyl group, or a carbamoyl group. R₂ represents one selected from a hydrogen atom, a halogen atom, an alkyl group, an alkoxy group, an aryloxy group, an alkylthio group, an arylthio group, an acyloxy group, or a carbonic acid ester group. R₃ and R₄ each independently represent a group substituting for a hydrogen atom on a benzene ring which is mentioned as the example of the substituent of formula (A-1). R₃ and R₄ may link together to form a condensed ring.

R₁ is preferably an alkyl group having 1 to 20 carbon atoms (for example, a methyl group, an ethyl group, an isopropyl group, a butyl group, a tert-octyl group, a cyclohexyl group, or the like), an acylamino group (for example, an acetylamino group, a benzoylamino group, a methylureido group, a 4-cyanophenylureido group, or the like), or a carbamoyl group (for example, a n-butylcarbamoyl group, an N,N-diethylcarbamoyl group, a phenylcarbamoyl group, a 2-chlorophenylcarbamoyl group, a 2,4-dichlorophenylcarbamoyl group, or the like). An acylamino group (including a ureido group and a urethane group) is more preferred. R₂ is preferably a halogen atom (more preferably, a chlorine atom or a bromine atom), an alkoxy group (for example, a methoxy group, a butoxy group, an n-hexyloxy group, an n-decyloxy group, a cyclohexyloxy group, a benzyloxy group, or the like), or an aryloxy group (for example, a phenoxy group, a naphthoxy group, or the like).

R₃ is preferably a hydrogen atom, a halogen atom, or an alkyl group having 1 to 20 carbon atoms, and most preferably a halogen atom. R₄ is preferably a hydrogen atom, an alkyl group, or an acylamino group, and more preferably an alkyl group or an acylamino group. Examples of the preferred substituent thereof are similar to those for R₁. In the case where R₄ is an acylamino group, R₄ may preferably link with R₃ to form a carbostyryl ring.

In the case where R₃ and R₄ in formula (A-2) link together to form a condensed ring, a naphthalene ring is particularly preferred as the condensed ring. The same substituent as the example of the substituent referred to for formula (A-1) may bond to the naphthalene ring. In the case where formula (A-2) is a naphthol compound, R₁ is preferably a carbamoyl group. Among them, a benzoyl group is particularly preferred. R₂ is preferably an alkoxy group or an aryloxy group, and particularly preferably an alkoxy group.

Preferred specific examples for the development accelerator according to the invention are to be described below. The invention is not restricted to these examples.

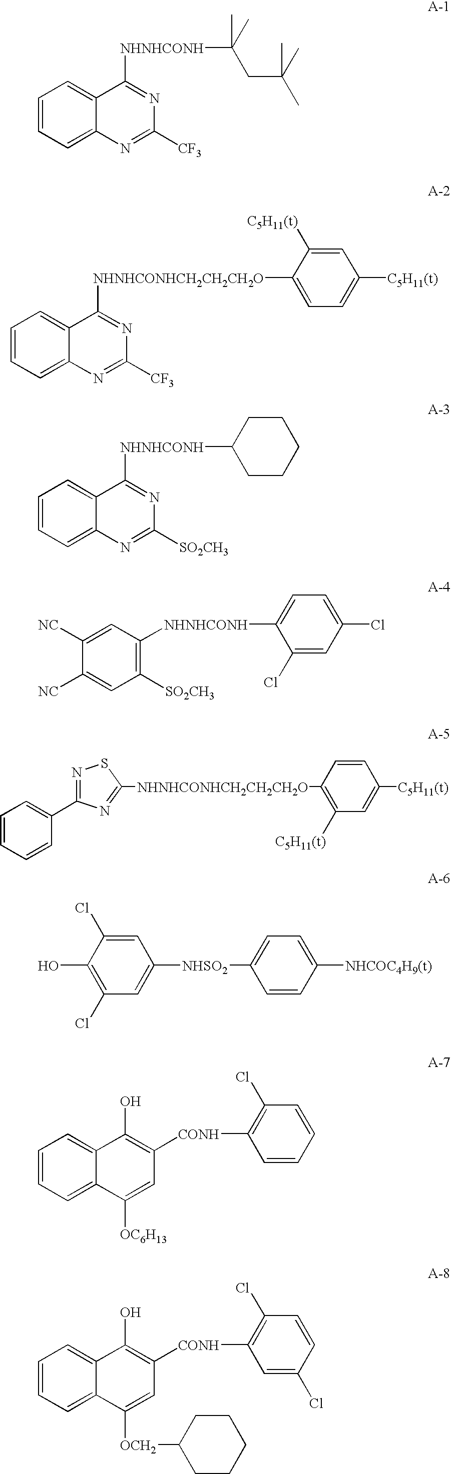

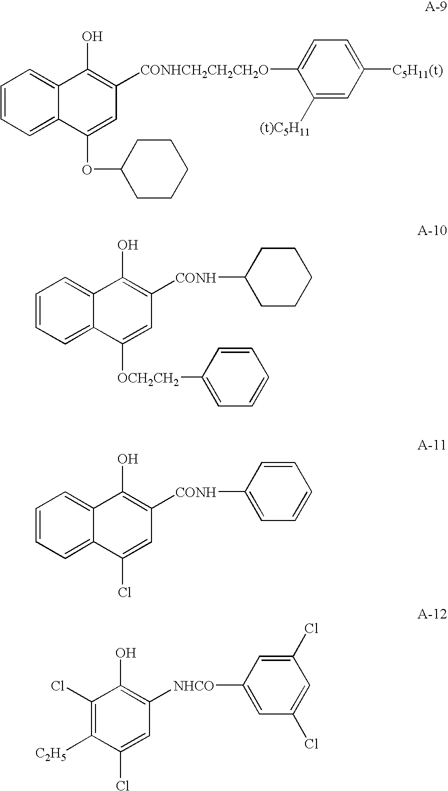

(Hydrogen Bonding Compound)

In the case where the reducing agent according to the invention has an aromatic hydroxy group (—OH) or an amino group (—NHR, R represents a hydrogen atom or an alkyl group), particularly in the case where the reducing agent is a bisphenol described above, it is preferred to use in combination a non-reducing compound having a group which forms a hydrogen bond with these groups of the reducing agent.

Examples of the group forming a hydrogen bond with the hydroxy group or amino group include a phosphoryl group, a sulfoxide group, a sulfonyl group, a carbonyl group, an amido group, an ester group, a urethane group, a ureido group, a tertiary amino group, a nitrogen-containing aromatic group, and the like. Preferred among them are a phosphoryl group, a sulfoxide group, an amido group (not having —N(H)— group but being blocked in the form of —N(Ra)— (where Ra represents a substituent other than H)), a urethane group (not having —N(H)— group but being blocked in the form of —N(Ra)— (where Ra represents a substituent other than H)), and a ureido group (not having —N(H)— group but being blocked in the form of —N(Ra)— (where Ra represents a substituent other than H)).

In the invention, particularly preferable hydrogen bonding compound is a compound represented by the following formula (D).

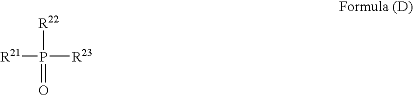

In formula (D), R²¹ to R²³ each independently represent one selected from an alkyl group, an aryl group, an alkoxy group, an aryloxy group, an amino group, or a heterocyclic group, which may be substituted or unsubstituted.

In the case where R²¹ to R²³ has a substituent, examples of the substituent include a halogen atom, an alkyl group, an aryl group, an alkoxy group, an amino group, an acyl group, an acylamino group, an alkylthio group, an arylthio group, a sulfonamido group, an acyloxy group, an oxycarbonyl group, a carbamoyl group, a sulfamoyl group, a sulfonyl group, a phosphoryl group, and the like, in which preferred as the substituent are an alkyl group and an aryl group, e.g., a methyl group, an ethyl group, an isopropyl group, a t-butyl group, a t-octyl group, a phenyl group, a 4-alkoxyphenyl group, a 4-acyloxyphenyl group, and the like.

Specific examples of the alkyl group represented by R²¹ to R²³ include a methyl group, an ethyl group, a butyl group, an octyl group, a dodecyl group, an isopropyl group, a t-butyl group, a t-amyl group, a t-octyl group, a cyclohexyl group, a 1-methylcyclohexyl group, a benzyl group, a phenethyl group, a 2-phenoxypropyl group, and the like.

Examples of the aryl group include a phenyl group, a cresyl group, a xylyl group, a naphthyl group, a 4-t-butylphenyl group, a 4-t-octylphenyl group, a 4-anisidyl group, a 3,5-dichlorophenyl group, and the like.

Examples of the alkoxy group include a methoxy group, an ethoxy group, a butoxy group, an octyloxy group, a 2-ethylhexyloxy group, a 3,5,5-trimethylhexyloxy group, a dodecyloxy group, a cyclohexyloxy group, a 4-methylcyclohexyloxy group, a benzyloxy group, and the like.

Examples of the aryloxy group include a phenoxy group, a cresyloxy group, an isopropylphenoxy group, a 4-t-butylphenoxy group, a naphthoxy group, a biphenyloxy group, and the like.

Examples of the amino group include a dimethylamino group, a diethylamino group, a dibutylamino group, a dioctylamino group, an N-methyl-N-hexylamino group, a dicyclohexylamino group, a diphenylamino group, an N-methyl-N-phenylamino group, and the like.

Preferred as R²¹ to R²³ are an alkyl group, an aryl group, an alkoxy group, and an aryloxy group. From the viewpoint of the effect of the invention, it is preferred that at least one of R²¹ to R²³ is an alkyl group or an aryl group, and it is more preferred that two or more of them are an alkyl group or an aryl group. Further, from the viewpoint of low cost availability, it is preferred that R²¹ to R²³ are of the same group.

Specific examples of the hydrogen bonding compound represented by formula (D) according to the invention and others are shown below, but the invention is not limited thereto.

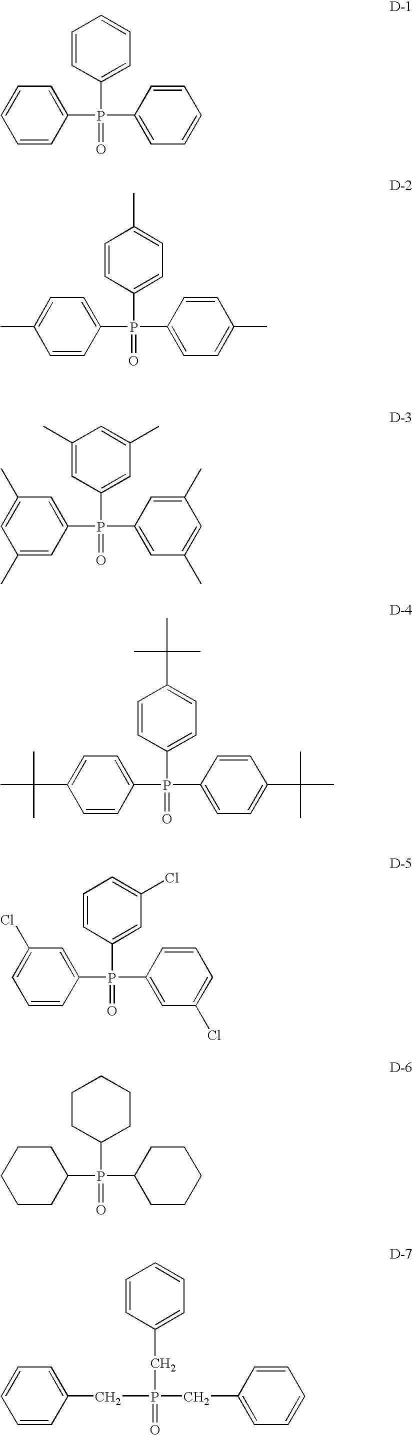

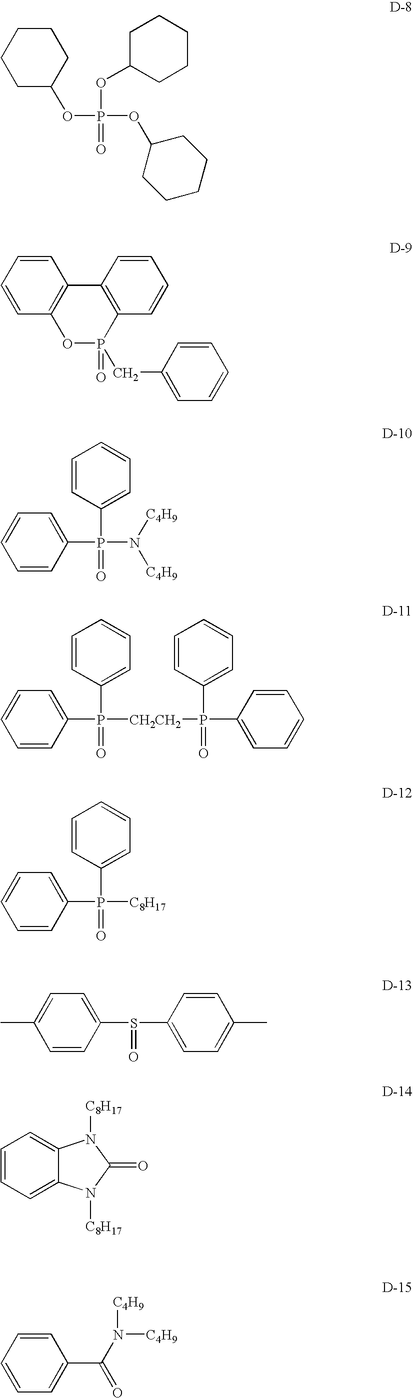

Specific examples of hydrogen bonding compounds other than those enumerated above can be found in those described in EP No. 1096310 and in JP-A Nos. 2002-156727 and 2002-318431.

The compound represented by formula (D) according to the invention can be used in the photothermographic material by being incorporated into the coating solution in the form of a solution, an emulsified dispersion, or a solid fine particle dispersion, similar to the case of reducing agent. However, it is preferably used in the form of a solid dispersion. In a solution state, the compound according to the invention forms a hydrogen-bonded complex with a compound having a phenolic hydroxy group or an amino group, and can be isolated as a complex in crystalline state depending on the combination of the reducing agent and the compound represented by formula (D) according to the invention.

It is particularly preferred to use the crystal powder thus isolated in the form of a solid fine particle dispersion, because it provides stable performance. Further, it is also preferred to use a method of leading to form complex during dispersion by mixing the reducing agent and the compound represented by formula (D) according to the invention in the form of powder, and dispersing them with a proper dispersing agent using sand grinder mill or the like.

The compound represented by formula (D) according to the invention is preferably used in a range of from 1 mol % to 200 mol %, more preferably from 10 mol % to 150 mol %, and even more preferably from 20 mol % to 100 mol %, with respect to the reducing agent.

(Silver Halide)

1) Halogen Composition

For the photosensitive silver halide used in the invention, there is no particular restriction on the halogen composition, and silver chloride, silver bromochloride, silver bromide, silver iodobromide, silver iodochlorobromide, or silver iodide can be used. Among these, silver bromide, silver iodobromide, and silver iodide are preferred. The distribution of the halogen composition in a grain may be uniform, the halogen composition may be changed stepwise, or it may be changed continuously. Further, a silver halide grain having a core/shell structure can be used preferably. Preferred structure is a twofold to fivefold structure, and more preferably, a core/shell grain having a twofold to fourfold structure can be used. Further, a technique of localizing silver bromide or silver iodide at the surface of a silver chloride, silver bromide, or silver chlorobromide grain can also be used preferably.

2) Method of Grain Formation

The method of forming photosensitive silver halide is well known in the relevant art and, for example, methods described in Research Disclosure No. 17029, June 1978 and U.S. Pat. No. 3,700,458 can be used. Specifically, a method of preparing a photosensitive silver halide by adding a silver-supplying compound and a halogen-supplying compound in a gelatin or other polymer solution and then mixing them with an organic silver salt is used. Further, a method described in JP-A No. 11-119374 (paragraph Nos. 0217 to 0224) and methods described in JP-A Nos. 11-352627 and 2000-347335 are also preferred.

3) Grain Size

The grain size of the photosensitive silver halide is preferably in a range of from 0.01 μm to 1.0 μm, more preferably from 0.03 μm to 0.5 μm, and even more preferably from 0.05 μm to 0.3 μm. The grain size as used herein means a diameter of a circle converted such that it has the same area as a projected area of the silver halide grain (projected area of a major plane in the case of a tabular grain).

4) Grain Shape

The shape of the silver halide grain includes, for example, cubic, octahedral, tabular, spherical, rod-like, and potato-like shape. A cubic grain is particularly preferred in the invention. A silver halide grain rounded at corners can also be used preferably. The surface indices (Miller indices) of the outer surface of a photosensitive silver halide grain are not particularly restricted, and it is preferable that the ratio occupied by the {100} face is large, because of showing high spectral sensitization efficiency when a spectral sensitizing dye is adsorbed. The ratio is preferably 50% or higher, more preferably 65% or higher, and even more preferably 80% or higher. The ratio of the {100} face, Miller indices, can be determined by a method utilizing adsorption dependency of the { 111 } face and { 100} face upon adsorption of a sensitizing dye, which is described in T. Tani; J. Imaging Sci., vol. 29, page 165, (1985).

5) Heavy Metal

The photosensitive silver halide grain according to the invention can contain metals or complexes of metals belonging to groups 6 to 13 of the periodic table (showing groups 1 to 18). Preferably, the photosensitive silver halide grain can contain metals or complexes of metals belonging to groups 6 to 10. The metal or the center metal of the metal complex from groups 6 to 10 of the periodic table is preferably rhodium, ruthenium, iridium, or ferrum. The metal complex may be used alone, or two or more complexes comprising identical or different species of metals may be used in combination. A preferred content is in a range of from 1×10⁻⁹ mol to 1×10⁻³ mol with respect to 1 mol of silver. The heavy metals, metal complexes, and the addition method thereof are described in JP-A No. 7-225449, in paragraph Nos. 0018 to 0024 of JP-A No. 11-65021, and in paragraph Nos. 0227 to 0240 of JP-A No. 11-119374.

In the present invention, a silver halide grain having a hexacyano metal complex present on the outermost surface of the grain is preferred. The hexacyano metal complex includes, for example, [Fe(CN)₆]⁴⁻, [Fe(CN)₆]³⁻, [Ru(CN)₆]⁴⁻, [Os(CN)₆]⁴⁻, [Co(CN)₆]³⁻, [Rh(CN)₆]³⁻, [Ir(CN)₆]³⁻, [Fe(CN)₆]³⁻, and [Re(CN)₆]³⁻. In the invention, hexacyano Fe complex is preferred.

Since the hexacyano metal complex exists in an ionic form in an aqueous solution, counter cation is not important, but an alkali metal ion such as sodium ion, potassium ion, rubidium ion, cesium ion, or lithium ion, ammonium ion, or an alkyl ammonium ion (for example, tetramethyl ammonium ion, tetraethyl ammonium ion, tetrapropyl ammonium ion, and tetra(n-butyl) ammonium ion), which are easily miscible with water and suitable to precipitation operation of silver halide emulsion, are preferably used.

The hexacyano metal complex can be added while being mixed with water, as well as a mixed solvent of water and an appropriate organic solvent miscible with water (for example, alcohols, ethers, glycols, ketones, esters, amides, or the like) or gelatin.

The addition amount of the hexacyano metal complex is preferably from 1×10⁻⁵ mol to 1×10⁻² mol, and more preferably from 1×10⁻⁴ mol to 1×10⁻³ mol, per 1 mol of silver.

In order to allow the hexacyano metal complex to be present on the outermost surface of a silver halide grain, the hexacyano metal complex is directly added in any stage of: after completion of addition of an aqueous solution of silver nitrate used for grain formation; before completion of an emulsion formation step prior to a chemical sensitization step of conducting chalcogen sensitization such as sulfur sensitization, selenium sensitization, or tellurium sensitization, or noble metal sensitization such as gold sensitization; during a washing step; during a dispersion step; and before a chemical sensitization step. In order not to grow fine silver halide grains, the hexacyano metal complex is preferably added rapidly after the grain is formed, and it is preferably added before completion of the emulsion formation step.

Addition of the hexacyano metal complex may be started after addition of 96% by weight of an entire amount of silver nitrate to be added for grain formation, more preferably started after addition of 98% by weight, and particularly preferably, started after addition of 99% by weight.

When any of the hexacyano metal complexes is added after addition of an aqueous solution of silver nitrate just prior to completion of grain formation, it can be adsorbed to the outermost surface of the silver halide grain, and most of the complexes form an insoluble salt with silver ions on the surface of the grain. Since silver hexacyanoferrate (II) is a salt less soluble than silver iodide, re-dissolution with fine grains can be prevented, and it becomes possible to prepare fine silver halide grains with smaller grain size.

Metal atoms that can be contained in the silver halide grain used in the invention (for example, [Fe(CN)₆]⁴⁻), and the desalting method and chemical sensitizing method of silver halide emulsion are described in paragraph Nos. 0046 to 0050 of JP-A No. 11-84574, in paragraph Nos. 0025 to 0031 of JP-A No. 11-65021, and in paragraph Nos. 0242 to 0250 of JP-A No. 11-119374.

6) Gelatin

As the gelatin which is contained in the photosensitive silver halide emulsion used in the invention, various types of gelatin can be used. It is necessary to maintain an excellent dispersion state of a photosensitive silver halide emulsion in the coating solution containing an organic silver salt, and gelatin having a molecular weight of 10,000 to 1,000,000 is preferably used. Phthalated gelatin is also preferably used. The gelatin may be used at the time of grain formation or at the time of dispersion after desalting treatment, and it is preferably used at the time of grain formation.

7) Sensitizing Dye

As the sensitizing dye which can be used in the invention, a sensitizing dye, which spectrally sensitizes the silver halide grains in a desired wavelength region upon adsorption to the silver halide grains and has spectral sensitivity suitable to the spectral characteristic of an exposure light source, can be advantageously selected. The sensitizing dyes and the addition method are described, for example, in paragraph Nos. 0103 to 0109 of JP-A No. 11-65021, as compounds represented by formula (II) in JP-A No. 10-186572, dyes represented by formula (I) and described in paragraph No. 0106 of JP-A No. 11-119374, dyes described in U.S. Pat. No. 5,510,236 and in the Example 5 of U.S. Pat. No. 3,871,887, dyes disclosed in JP-A Nos. 2-96131 and 59-48753, as well as in page 19, line 38 to page 20, line 35 of EP No. 0803764A1, and in JP-A Nos. 2001-272747, 2001-290238 and 2002-23306, and the like. The sensitizing dye may be used alone, or two or more of them may be used in combination. In the invention, the sensitizing dye is preferably added in the silver halide emulsion within a period after desalting step until coating, and more preferably in a period after desalting step until the completion of chemical ripening.

In the invention, the sensitizing dye may be added at any amount according to the property of sensitivity or fogging, but it is preferably added in an amount of from 10⁻⁶ mol to 1 mol, and more preferably from 10⁻⁴ mol to 10⁻¹ mol, per 1 mol of photosensitive silver halide.

In the invention, a super sensitizer can be used in order to improve the spectral sensitizing effect. The super sensitizer that can be used in the invention includes those compounds described in EP-A No. 587,338, U.S. Pat. Nos. 3,877,943 and 4,873,184, JP-A Nos. 5-341432, 11-109547, and 10-111543, and the like.

8) Chemical Sensitization

The photosensitive silver halide grain according to the invention is preferably chemically sensitized by sulfur sensitizing method, selenium sensitizing method, or tellurium sensitizing method. As the compounds used preferably for sulfur sensitizing method, selenium sensitizing method, and tellurium sensitizing method, known compounds, for example, compounds described in JP-A No. 7-128768 and the like can be used. Particularly, tellurium sensitization is preferred in the invention, and compounds described in the literature cited in paragraph No. 0030 in JP-A No. 11-65021 and compounds represented by formula (II), (III), or (IV) in JP-A No. 5-313284 are more preferred.

The photosensitive silver halide grain in the invention is preferably chemically sensitized by gold sensitizing method alone or in combination with the chalcogen sensitization described above. As the gold sensitizer, those having an oxidation number of gold of either +1 or +3 are preferred, and those gold compounds usually used as the gold sensitizer are preferred. As typical examples, chloroauric acid, bromoauric acid, potassium chloroaurate, potassium bromoaurate, auric trichloride, potassium auric thiocyanate, potassium iodoaurate, tetracyanoauric acid, ammonium aurothiocyanate, and pyridyl trichloro gold are preferred. Further, gold sensitizers described in U.S. Pat. No. 5,858,637 and JP-A No. 2002-278016 are also used preferably.

In the invention, chemical sensitization can be applied at any time so long as it is after grain formation and before coating, and it can be applied, after desalting, (1) before spectral sensitization, (2) simultaneously with spectral sensitization, (3) after spectral sensitization, (4) just prior to coating, or the like.

The amount of sulfur, selenium, or tellurium sensitizer used in the invention may vary depending on the silver halide grain used, the chemical ripening condition, and the like, and it is used in an amount of from 10⁻⁸ mol to 10⁻² mol, and preferably from 10⁻⁷ mol to 10⁻³ mol, per 1 mol of silver halide.

The addition amount of the gold sensitizer may vary depending on various conditions, but it is generally from 10⁻⁷ mol to 10⁻³ mol, and preferably from 10⁻⁶ mol to 5×10⁻⁴ mol, per 1 mol of silver halide.

There is no particular restriction on the conditions for the chemical sensitization in the invention, and appropriately, the pH is from 5 to 8, the pAg is from 6 to 11, and the temperature is from 40° C. to 95° C.

In the silver halide emulsion used in the invention, a thiosulfonic acid compound may be added by the method shown in EP-A No. 293,917.

A reduction sensitizer is preferably used for the photosensitive silver halide grain according to the invention. As the specific compound for the reduction sensitizing method, ascorbic acid or aminoimino methane sulfinic acid is preferred, as well as the use of stannous chloride, a hydrazine derivative, a borane compound, a silane compound, or a polyamine compound is preferred. The reduction sensitizer may be added at any stage in the photosensitive emulsion production process from crystal growth to the preparation step just prior to coating. Further, it is preferred to apply reduction sensitization by ripening while keeping the pH to 7 or higher or the pAg to 8.3 or lower for the emulsion, and it is also preferred to apply reduction sensitization by introducing a single addition portion of silver ions during grain formation.

9) Compound that is One-Electron-Oxidized to Provide a One-Electron Oxidation Product Which Releases One or More Electrons

The photothermographic material according to the present invention preferably contains a compound that is one-electron-oxidized to provide a one-electron oxidation product which releases one or more electrons. The said compound can be used alone or in combination with various chemical sensitizers described above to increase the sensitivity of silver halide.

The compound that is one-electron-oxidized to provide a one-electron oxidation product which releases one or more electrons, which is contained in the photothermographic material according to the invention, is a compound selected from the following Groups 1 or 2.

(Group 1) a compound that is one-electron-oxidized to provide a one-electron oxidation product which further releases one or more electrons due to being subjected to a subsequent bond cleavage reaction;

(Group 2) a compound that is one-electron-oxidized to provide a one-electron oxidation product which further releases one or more electrons after being subjected to a subsequent bond formation reaction.

The compound of Group 1 will be explained below.

In the compound of Group 1, as a compound that is one-electron-oxidized to provide a one-electron oxidation product which further releases one electron due to being subjected to a subsequent bond cleavage reaction, specific examples include examples of compound referred to as “one photon two electrons sensitizer” or “deprotonating electron-donating sensitizer” described in JP-A No. 9-211769 (specific examples: Compound PMT-1 to S-37 in Tables E and F, pages 28 to 32); JP-A No. 9-211774; JP-A No. 11-95355 (specific examples: Compound INV 1 to 36); JP-W No. 2001-500996 (specific examples: Compound 1 to 74, 80 to 87, and 92 to 122); U.S. Pat. Nos. 5,747,235 and 5,747,236; EP No. 786692A1 (specific examples: Compound INV 1 to 35); EP No. 893732A1; U.S. Pat. Nos. 6,054,260 and 5,994,051; etc. Preferred ranges of these compounds are the same as the preferred ranges described in the quoted specifications.

In the compound of Group 1, as a compound that is one-electron-oxidized to provide a one-electron oxidation product which further releases one or more electrons due to being subjected to a subsequent bond cleavage reaction, specific examples include the compounds represented by formula (1) (same as formula (1) described in JP-A No. 2003-114487), formula (2) (same as formula (2) described in JP-A No. 2003-114487), formula (3) (same as formula (1) described in JP-A No. 2003-114488), formula (4) (same as formula (2) described in JP-A No. 2003-114488), formula (5) (same as formula (3) described in JP-A No. 2003-114488), formula (6) (same as formula (1) described in JP-A No. 2003-75950), formula (7) (same as formula (2) described in JP-A No. 2003-75950), and formula (8) (same as formula (1) described in JP-A No. 2004-239943), and the compound represented by formula (9) (same as formula (3) described in JP-A No. 2004-245929) among the compounds which can undergo the reaction represented by chemical reaction formula (1) (same as chemical reaction formula (1) described in JP-A No. 2004-245929). Preferable ranges of these compounds are the same as the preferable ranges described in the quoted specifications.

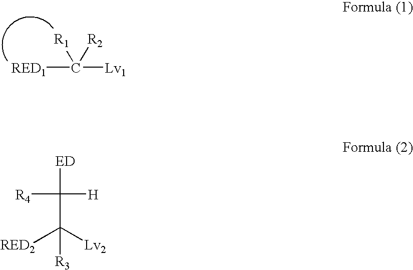

In formulae (1) and (2), RED₁ and RED₂ each independently represent a reducing group. R₁ represents a nonmetallic atomic group forming a cyclic structure equivalent to a tetrahydro derivative or hexahydro derivative of a 5- or 6-membered aromatic ring (including an aromatic heterocycle) with the carbon atom (C) and RED₁.

R₂, R₃, and R₄ each independently represent a hydrogen atom or a substituent. Lv₁ and Lv₂ each independently represent a leaving group. ED represents an electron-donating group.

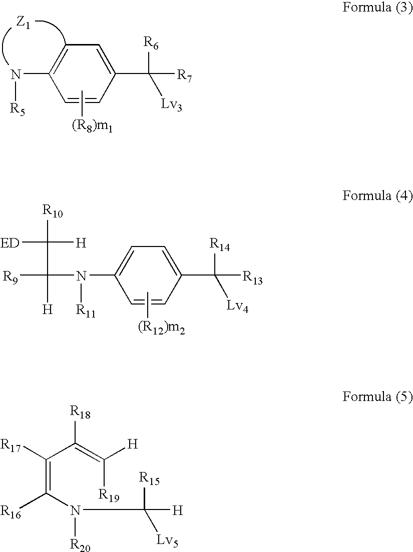

In formulae (3), (4), and (5), Z₁ represents an atomic group forming a 6-membered ring with a nitrogen atom and two carbon atoms of the benzene ring. R₅, R₆, R₇, R₉, R₁₀, R₁₁, R¹³, R₁₄, R₁₅, R₁₆, R₁₇, R₁₈, and R₁₉ each independently represent a hydrogen atom or a substituent. R₂₀ represents a hydrogen atom or a substituent; however, in the case where R₂₀ represents a group other than an aryl group, R₁₆ and R₁₇ bond to each other to form an aromatic ring or an aromatic heterocycle. R₈ and R₁₂ represent a substituent which substitutes for a hydrogen atom on a benzene ring. m₁ represents an integer of from 0 to 3, and m2 represents an integer of from 0 to 4. Lv₃, Lv₄, and Lv₅ each independently represent a leaving group.

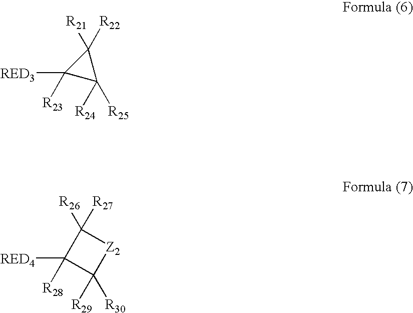

In formulae (6) and (7), RED₃ and RED₄ each independently represent a reducing group. R₂₁ to R₃₀ each independently represent a hydrogen atom or a substituent. Z₂ represents —CR₁₁₁R₁₁₂—, —NR₁₁₃—, or —O—. R₁₁₁, and R₁₁₂ each independently represent a hydrogen atom or a substituent. R₁₁₃ represents a hydrogen atom, an alkyl group, an aryl group, or a heterocyclic group.

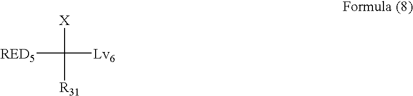

In formula (8), RED₅ is a reducing group and represents an arylamino group or a heterocyclic amino group. R₃₁ represents a hydrogen atom or a substituent. X represents one selected from an alkoxy group, an aryloxy group, a heterocyclic oxy group, an alkylthio group, an arylthio group, a heterocyclic thio group, an alkylamino group, an arylamino group, or a heterocyclic amino group. Lv₆ is a leaving group and represents a carboxy group or a salt thereof, or a hydrogen atom.

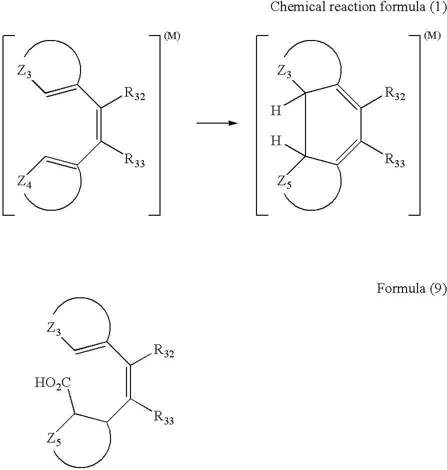

The compound represented by formula (9) is a compound that undergoes a bond formation reaction represented by chemical reaction formula (1) after undergoing two-electrons-oxidation accompanied by decarboxylation and further oxidized. In chemical reaction formula (1), R₃₂ and R₃₃ represent a hydrogen atom or a substituent. Z₃ represents a group which forms a 5- or 6-membered heterocycle with C═C. Z₄ represents a group which forms a 5- or 6-membered aryl group or heterocyclic group with C═C. M represents a radical, a radical cation, or a cation. In formula (9), R₃₂, R₃₃, and Z₃ each have the same meaning as in chemical reaction formula (1). Z₅ represents a group which forms a 5- or 6-membered cyclic aliphatic hydrocarbon group or heterocyclic group with C—C.

Next, the compound of Group 2 is explained.

In the compound of Group 2, as a compound that is one-electron-oxidized to provide a one-electron oxidation product which further releases one or more electrons after being subjected to a subsequent bond formation reaction, specific examples can include the compound represented by formula (10) (same as formula (1) described in JP-A No. 2003-140287), and the compound represented by formula (11) (same as formula (2) described in JP-A No. 2004-245929) which can undergo the reaction represented by chemical reaction formula (1) (same as chemical reaction formula (1) described in JP-A No. 2004-245929). The preferable ranges of these compounds are the same as the preferable ranges described in the quoted specifications.

RED₆-Q-Y

In formula (10), RED₆ represents a reducing group which is to be one-electron-oxidized. Y represents a reactive group containing a carbon-carbon double bond part, a carbon-carbon triple bond part, an aromatic group part, or a benzo-condensed non-aromatic heterocycle part, which reacts with one-electron-oxidized product formed by one-electron-oxidation of RED₆ to form a new bond. Q represents a linking group which links RED₆ and Y.

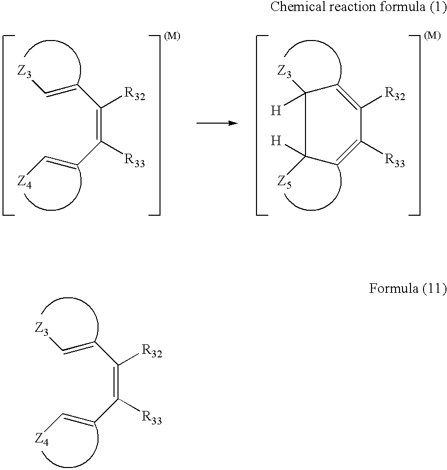

The compound represented by formula (11) is a compound that undergoes a bond formation reaction represented by chemical reaction formula (1) by being oxidized. In chemical reaction formula (1), R₃₂ and R₃₃ each independently represent a hydrogen atom or a substituent. Z₃ represents a group which forms a 5- or 6-membered heterocycle with C═C. Z₄ represents a group which forms a 5- or 6-membered aryl group or heterocyclic group with C═C. Z₅ represents a group which forms a 5- or 6-membered cyclic aliphatic hydrocarbon group or heterocyclic group with C—C. M represents a radical, a radical cation, or a cation. In formula (11), R₃₂, R₃₃, Z₃, and Z₄ each have the same meaning as in chemical reaction formula (1).

The compounds of Groups 1 or 2 are preferably “the compound having an adsorptive group to silver halide in the molecule” or “the compound having a partial structure of a spectral sensitizing dye in the molecule”. The representative adsorptive group to silver halide is the group described in JP-A No. 2003-156823, page 16 right, line 1 to page 17 right, line 12. The partial structure of a spectral sensitizing dye is the structure described in the same specification, page 17 right, line 34 to page 18 right, line 6.

As the compound of Groups 1 or 2, “the compound having at least one adsorptive group to silver halide in the molecule” is more preferred, and “the compound having two or more adsorptive groups to silver halide in the same molecule” is even more preferred. In the case where two or more adsorptive groups exist in a single molecule, those adsorptive groups may be identical or different from one another.

As preferable adsorptive group, a mercapto-substituted nitrogen-containing heterocyclic group (e.g., a 2-mercaptothiadiazole group, a 3-mercapto-1,2,4-triazole group, a 5-mercaptotetrazole group, a 2-mercapto-1,3,4-oxadiazole group, a 2-mercaptobenzoxazole group, a 2-mercaptobenzothiazole group, a 1,5-dimethyl-1,2,4-triazolium-3-thiolate group, or the like) or a nitrogen-containing heterocyclic group having an —NH— group, which forms silver iminate (—N(Ag)—), as a partial structure of the heterocycle (e.g., a benzotriazole group, a benzimidazole group, an indazole group., or the like) are described. A 5-mercaptotetrazole group, a 3-mercapto-1,2,4-triazole group, and a benzotriazole group are particularly preferable, and a 3-mercapto-1,2,4-triazole group and a 5-mercaptotetrazole group are most preferable.

The case where the adsorptive group has two or more mercapto groups as a partial structure in the molecule is also particularly preferable. Herein, the mercapto group (—SH) may become a thione group in the case where it can tautomerize. Preferred examples of the adsorptive group having two or more mercapto groups as a partial structure (dimercapto-substituted nitrogen-containing heterocyclic group and the like) include a 2,4-dimercaptopyrimidine group, a 2,4-dimercaptotriazine group, and a 3,5-dimercapto-1,2,4-triazole group.

Further, a quaternary salt structure of nitrogen or phosphorus is also preferably used as the adsorptive group. Specific examples of the quaternary salt structure of nitrogen include an ammonio group (a trialkylammonio group, a dialkylarylammonio group, a dialkylheteroarylammonio group, an alkyldiarylammonio group, an alkyldiheteroarylammonio group, or the like) and a nitrogen-containing heterocyclic group containing a quaternary nitrogen atom. Examples of the quaternary salt structure of phosphorus include a phosphonio group (a trialkylphosphonio group, a dialkylarylphosphonio group, a dialkylheteroarylphosphonio group, an alkyldiarylphosphonio group, an alkyldiheteroarylphosphonio group, a triarylphosphonio group, a triheteroarylphosphonio group, or the like). A quaternary salt structure of nitrogen is more preferably used, and a 5- or 6-membered nitrogen-containing aromatic heterocyclic group containing a quaternary nitrogen atom is even more preferably used: Particularly preferably, a pyridinio group, a quinolinio group, or an isoquinolinio group is used. These nitrogen-containing heterocyclic groups containing a quaternary nitrogen atom may have any substituent.

Examples of a counter anion of the quaternary salt include a halogen ion, carboxylate ion, sulfonate ion, sulfate ion, perchlorate ion, carbonate ion, nitrate ion, BF₄⁻, PF₆⁻, Ph₄B⁻, and the like. In the case where the group having negative charge at carboxylate group or the like exists in the molecule, an inner salt may be formed with it. As a counter anion outside of the molecule, chloro ion, bromo ion, or methanesulfonate ion is particularly preferable.

Preferred structure of the compound represented by Groups 1 or 2 having a quaternary salt structure of nitrogen or phosphorus as the adsorptive group is represented by formula (X).

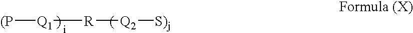

In formula (X), P and R each independently represent a quaternary salt structure of nitrogen or phosphorus, which is not a partial structure of a spectral sensitizing dye. Q₁ and Q₂ each independently represent a linking group and typically represent a single bond, an alkylene group, an arylene group, a heterocyclic group, —O—, —S—, —NR_{N}, —C(═O)—, —SO₂—, —SO—, —P(═O)— or combinations of these groups. Herein, R_{N} represents a hydrogen atom, an alkyl group, an aryl group, or a heterocyclic group. S represents a residue which is obtained by removing one atom from the compound represented by Group 1 or 2. i and j are an integer of one or more and are selected from within a range satisfying i+j=2 to 6. The case where i is 1 to 3 and j is 1 to 2 is preferable, the case where i is 1 or 2 and j is 1 is more preferable, and the case where i is 1 and j is 1 is particularly preferable. The compound represented by formula (X) preferably has 10 to 100 carbon atoms in total, more preferably 10 to 70 carbon atoms, even more preferably 11 to 60 carbon atoms, and particularly preferably 12 to 50 carbon atoms in total.

Specific examples of the compound represented by Group 1 or Group 2 are shown below, but the invention is not limited to these examples.

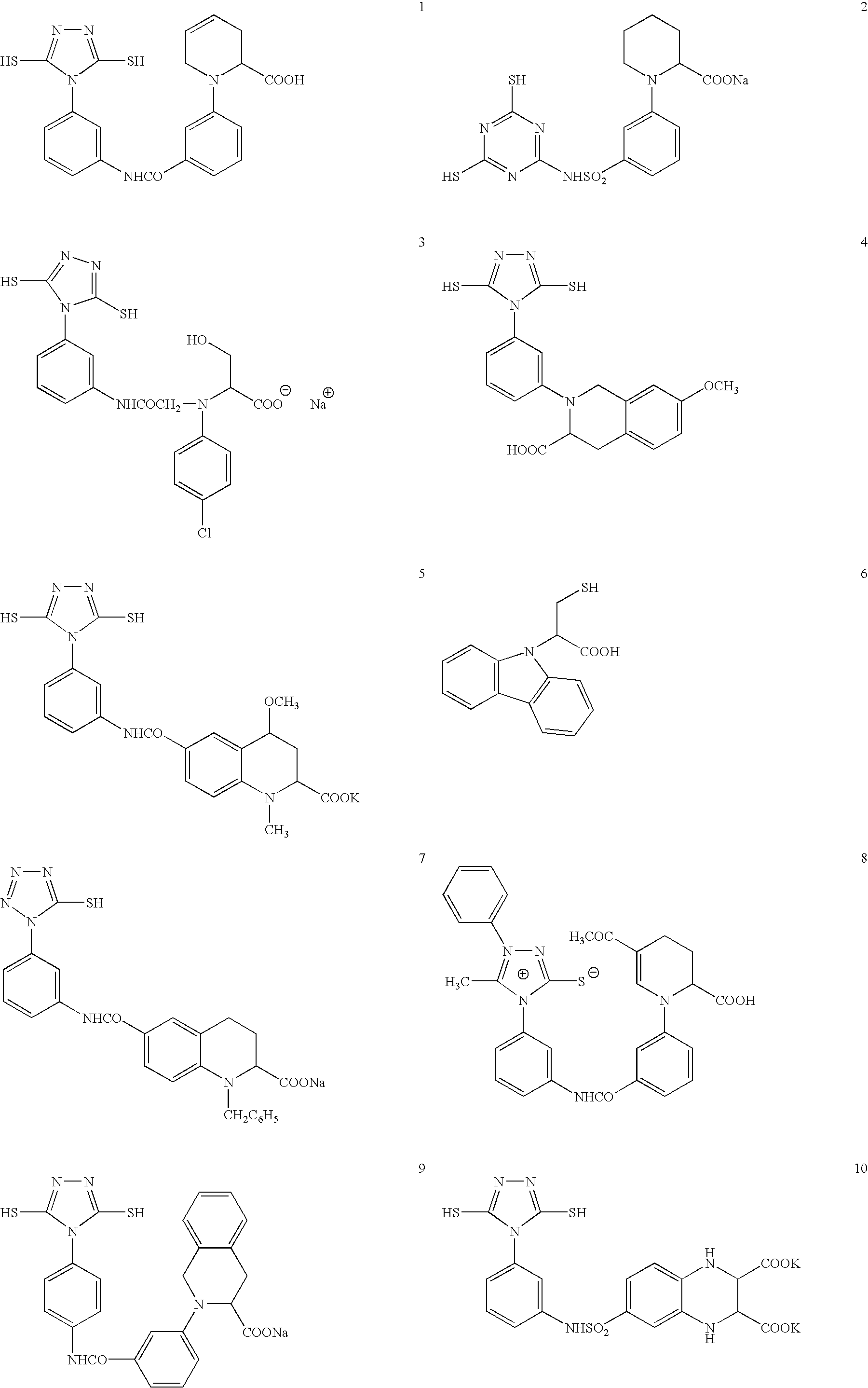

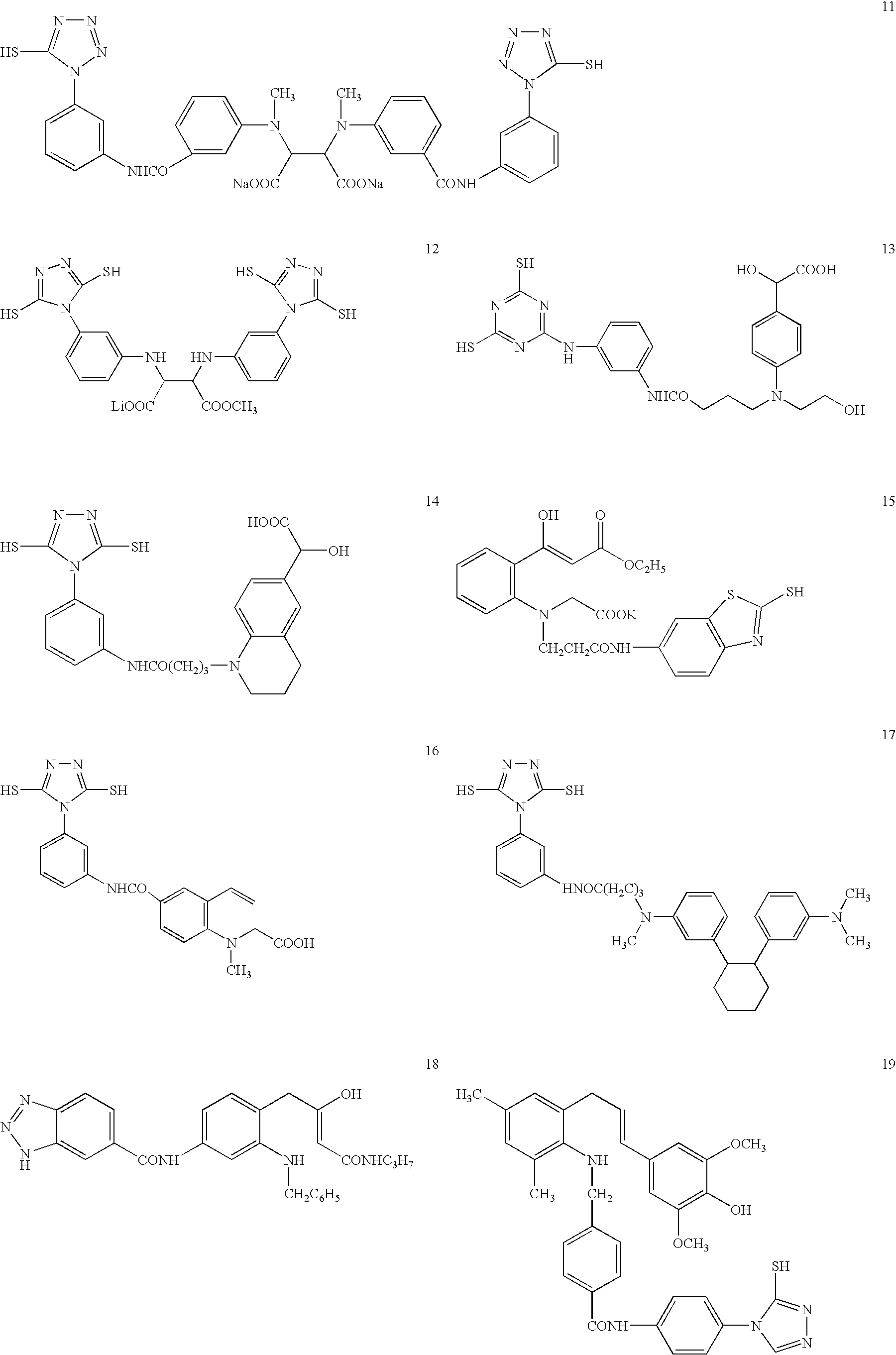

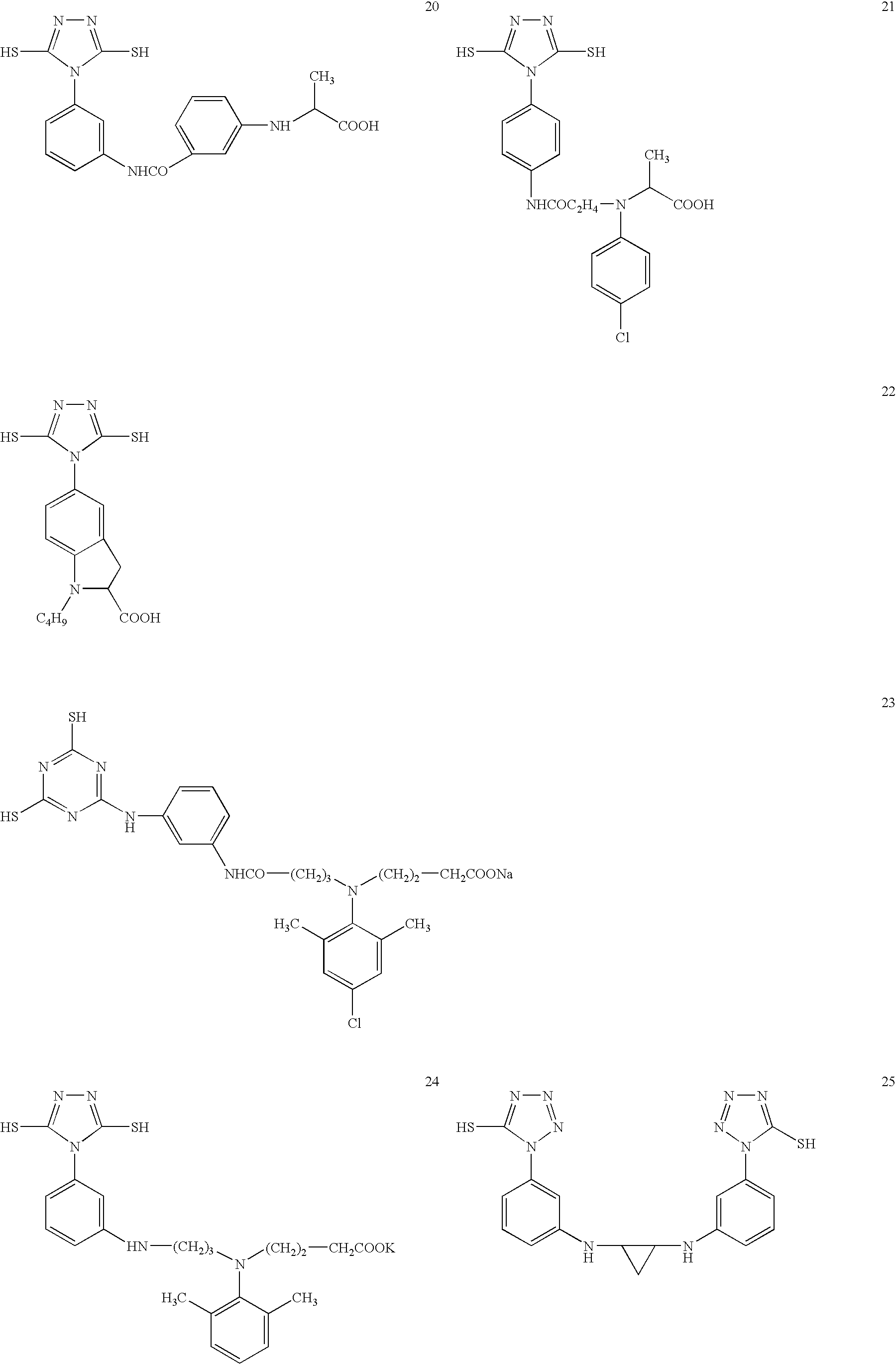

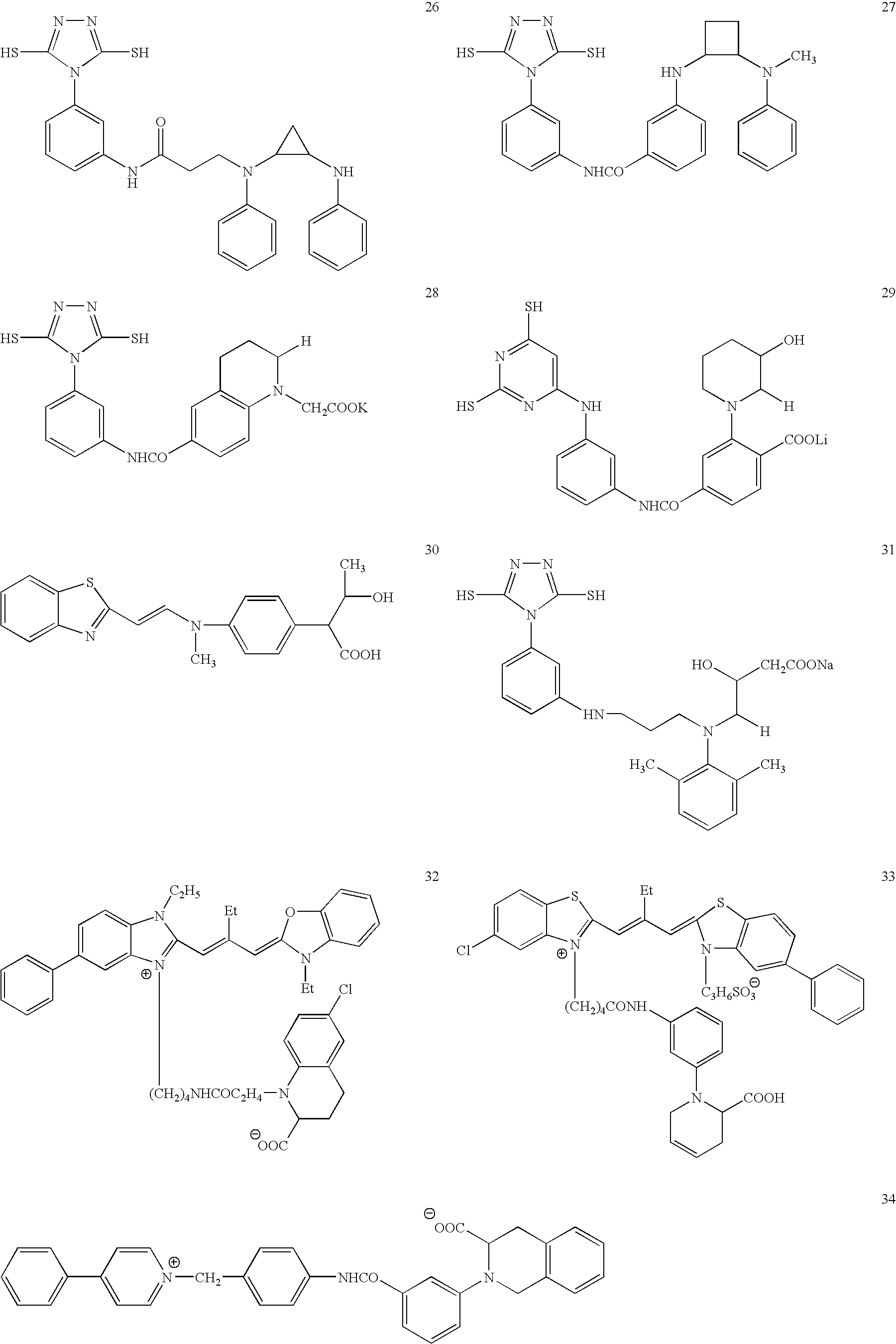

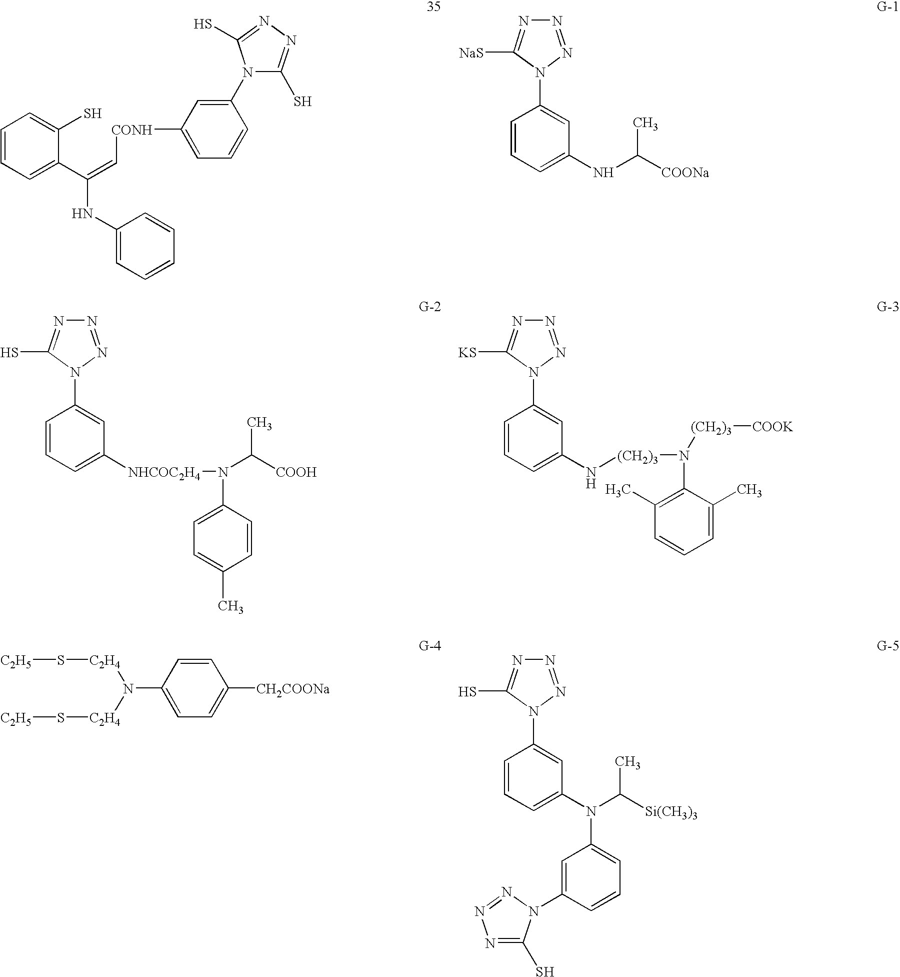

The compounds of Groups 1 or 2 may be used at any time during preparation of the photosensitive silver halide emulsion and production of the photothermographic material. For example, the compound may be used in a photosensitive silver halide grain formation step, in a desalting step, in a chemical sensitization step, before coating, or the like. The compound may be added several times during these steps. The compound is preferably added after completion of the photosensitive silver halide grain formation step and before the desalting step; in the chemical sensitization step (just before initiation of the chemical sensitization to immediately after completion of the chemical sensitization); or before coating. The compound is more preferably added within a period from the chemical sensitization to before being mixed with the non-photosensitive organic silver salt.

It is preferred that the compound of Groups 1 or 2 according to the invention is added by being dissolved in water, a water-soluble solvent such as methanol or ethanol, or a mixed solvent thereof. In the case where the compound is dissolved in water and solubility of the compound is increased by increasing or decreasing a pH value of the solvent, the pH value may be increased or decreased to dissolve and add the compound.

The compound of Groups 1 or 2 according to the invention is preferably used in the image forming layer which contains the photosensitive silver halide and the non-photosensitive organic silver salt. The compound may be added to a protective layer or intermediate layer, as well as the image forming layer containing the photosensitive silver halide and the non-photosensitive organic silver salt, to be diffused in the coating step. The compound may be added before or after addition of a sensitizing dye. The compound is contained in the silver halide emulsion layer (image forming layer) preferably in an amount of from 1×10⁻⁹ mol to 5×10⁻¹ mol, and more preferably from 1×10⁻⁻⁸ mol to 5×10⁻² mol, per 1 mol of silver halide.

10) Compound Having Adsorptive Group and Reducing Group

The photothermographic material according to the present invention preferably contains a compound having an adsorptive group to silver halide and a reducing group in the molecule. It is preferred that the compound is represented by the following formula (I).

A-(W)n-B Formula (I)

In formula (I), A represents a group which adsorbs to a silver halide (hereafter, it is called an adsorptive group.); W represents a divalent linking group; n represents 0 or 1; and B represents a reducing group.

In formula (I), the adsorptive group represented by A is a group to adsorb directly to a silver halide or a group to promote adsorption to a silver halide. As typical examples, a mercapto group (or a salt thereof); a thione group (—C(═S)—); a heterocyclic group comprising at least one atom selected from among nitrogen, sulfur, selenium, and tellurium; a sulfide group; a disulfide group; a cationic group; an ethynyl group; and the like are described.

The mercapto group (or the salt thereof) as the adsorptive group means a mercapto group (or a salt thereof) itself and simultaneously more preferably represents a heterocyclic group, aryl group, or alkyl group substituted by at least one mercapto group (or a salt thereof). Herein, the heterocyclic group is at least a 5- to 7-membered, monocyclic or condensed, aromatic or non-aromatic heterocyclic group; and examples thereof include an imidazole ring group, a thiazole ring group, an oxazole ring group, a benzimidazole ring group, a benzothiazole ring group, a benzoxazole ring group, a triazole ring group, a thiadiazole ring group, an oxadiazole ring group, a tetrazole ring group, a purine ring group, a pyridine ring group, a quinoline ring group, an isoquinoline ring group, a pyrimidine ring group, a triazine ring group, and the like. A heterocyclic group having a quaternary nitrogen atom may also be adopted, wherein the mercapto group as a substituent may dissociate to form a mesoion. When the mercapto group forms a salt, a counter ion of the salt may be a cation of an alkaline metal, alkaline earth metal, heavy metal, or the like, such as Li⁺, Na⁺, K⁺, Mg²⁺, Ag⁺, or Zn²⁺; an ammonium ion; a heterocyclic group containing a quaternary nitrogen atom; a phosphonium ion, or the like.

Further, the mercapto group as the adsorptive group may become a thione group by tautomerization.

The thione group used as the adsorptive group also includes a chain or cyclic thioamido group, thioureido group, thiourethane group, and dithiocarbamic acid ester group.

The heterocyclic group, as the adsorptive group, which comprises at least one atom selected from among nitrogen, sulfur, selenium, and tellurium, represents a nitrogen-containing heterocyclic group having an —NH— group, which forms silver iminate (—N(Ag)—), as a partial structure of the heterocycle, or a heterocyclic group having an —S— group, —Se— group, —Te— group, or ═N— group, which coordinates to a silver ion by a coordination bond, as a partial structure of the heterocycle. As the former examples, a benzotriazole group, a triazole group, an indazole group, a pyrazole group, a tetrazole group, a benzimidazole group, an imidazole group, a purine group, and the like are described. As the latter examples, a thiophene group, a thiazole group, an oxazole group, a benzothiophene group, a benzothiazole group, a benzoxazole group, a thiadiazole group, an oxadiazole group, a triazine group, a selenoazole group, a benzoselenoazole group, a tellurazole group, a benzotellurazole group, and the like are described.

The sulfide group or disulfide group as the adsorptive group contains all groups having “—S—” or “—S—S—” as a partial structure.

The cationic group as the adsorptive group means a group containing a quaternary nitrogen atom, such as an ammonio group or a nitrogen-containing heterocyclic group containing a quaternary nitrogen atom. As examples of the nitrogen-containing heterocyclic group containing a quaternary nitrogen atom, a pyridinio group, a quinolinio group, an isoquinolinio group, an imidazolio group, and the like are described.

The ethynyl group as the adsorptive group means —C≡CH group and the said hydrogen atom may be substituted.

The adsorptive group described above may have any substituent.

Further, as typical examples of the adsorptive group, the groups described in pages 4 to 7 in the specification of JP-A No. 11-95355 are described.

As the adsorptive group represented by A in formula (I), a mercapto-substituted heterocyclic group (for example, a 2-mercaptothiadiazole group, a 2-mercapto-5-aminothiadiazole group, a 3-mercapto-1,2,4-triazole group, a 5-mercaptotetrazole group, a 2-mercapto-1,3,4-oxadiazole group, a 2-mercaptobenzimidazole group, a 1,5-dimethyl-1,2,4-triazolium-3-thiolate group, a 2,4-dimercaptopyrimidine group, a 2,4-dimercaptotriazine group, a 3,5-dimercapto-1,2,4-triazole group, a 2,5-dimercapto-1,3-thiazole group, or the like) and a nitrogen-containing heterocyclic group having an —NH— group, which forms silver iminate (—N(Ag)—), as a partial structure of the heterocycle (for example, a benzotriazole group, a benzimidazole group, an indazole group, or the like) are preferable, and more preferable as the adsorptive group are a 2-mercaptobenzimidazole group and a 3,5-dimercapto-1,2,4-triazole group.

In formula (I), W represents a divalent linking group. The said linking group may be any divalent linking group as long as it does not exert adverse influences on photographic performance. For example, a divalent linking group which is formed from carbon, hydrogen, oxygen, nitrogen, or sulfur can be used. Specific examples thereof include an alkylene group having 1 to 20 carbon atoms (for example, a methylene group, an ethylene group, a trimethylene group, a tetramethylene group, a hexamethylene group, or the like), an alkenylene group having 2 to 20 carbon atoms, an alkynylene group having 2 to 20 carbon atoms, an arylene group having 6 to 20 carbon atoms (for example, a phenylene group, a naphthylene group, or the like), —CO—, —SO₂—, —O—, —S—, —NR₁—, and combinations of these linking groups. Herein, R₁ represents a hydrogen atom, an alkyl group, a heterocyclic group, or an aryl group.

The linking group represented by W may have any substituent.

In formula (I), the reducing group represented by B represents a group which reduces a silver ion. Examples thereof include a formyl group; an amino group; a triple bond group such as an acetylene group, a propargyl group, or the like; a mercapto group; and residues which are obtained by removing one hydrogen atom from hydroxyamines, hydroxamic acids, hydroxyureas, hydroxyurethanes, hydroxysemicarbazides, reductones (reductone derivatives are contained), anilines, phenols(chroman-6-ols, 2,3-dihydrobenzofuran-5-ols, aminophenols, sulfonamidophenols, and polyphenols such as hydroquinones, catechols, resorcinols, benzenetriols, bisphenols are included), acylhydrazines, carbamoylhydrazines, 3-pyrazolidones, and the like. They may have any substituent.

The oxidation potential of the reducing group represented by B in formula (I) can be measured by using the measuring method described in Akira Fujishima, “DENKIKAGAKU SOKUTEIHO”, pages 150 to 208, GIHODO SHUPPAN and The Chemical Society of Japan, “JIKKEN KAGAKU KOZA”, 4th ed., vol. 9, pages 282 to 344, MARUZEN. For example, the method of rotating disc voltammetry can be used; namely the sample is dissolved in the solution (methanol : pH 6.5 Britton-Robinson buffer=10%: 90% (% by volume)) and after bubbling with nitrogen gas for 10 minutes, the voltamograph can be measured under conditions of 1000 rotations/minute, sweep rate of 20 mV/second, at 25° C. by using a rotating disc electrode (RDE) made by glassy carbon as a working electrode, a platinum electrode as a counter electrode, and a saturated calomel electrode as a reference electrode. The half wave potential (E1/2) can be calculated by that obtained voltamograph.

When the reducing group represented by B in the present invention is measured by the method described above, the oxidation potential is preferably in a range of from about −0.3 V to about 1.0 V, more preferably from about −0.1 V to about 0.8 V, and particularly preferably from about 0 V to about 0.7 V.

In formula (I), the reducing group represented by B is preferably a residue which is obtained by removing one hydrogen atom from hydroxyamines, hydroxamic acids, hydroxyureas, hydroxysemicarbazides, reductones, phenols, acylhydrazines, carbamoylhydrazines, or 3-pyrazolidones.

The compound of formula (I) according to the present invention may have a ballast group or polymer chain, which are generally used in the non-moving photographic additives such as a coupler or the like, in it. And as the polymer, for example, the polymer described in JP-A No. 1-100530 is described.

The compound of formula (I) according to the present invention may be bis or tris type of compound. The molecular weight of the compound represented by formula (I) according to the present invention is preferably within a range of from 100 to 10000, more preferably from 120 to 1000, and particularly preferably from 150 to 500.

Specific examples of the compound represented by formula (I) according to the present invention are shown below, but the present invention is not limited to these examples.

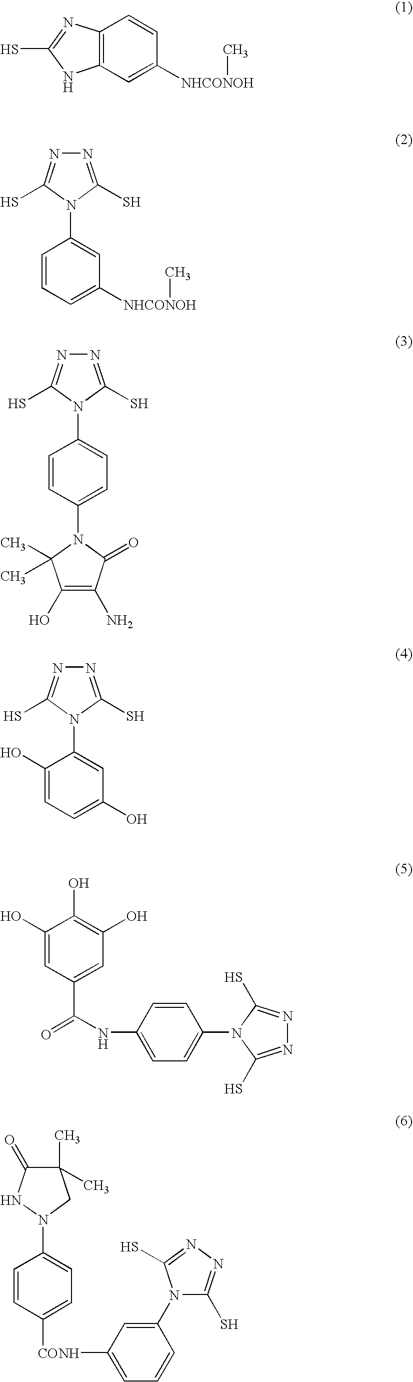

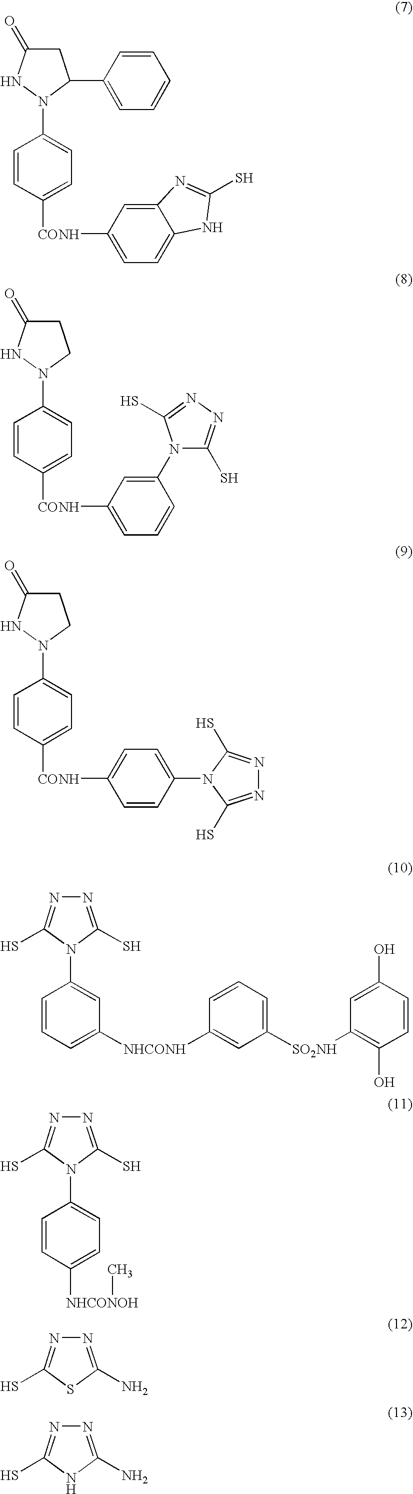

Further, specific compounds 1 to 30 and 1″-1 to 1″-77 shown in EP No. 1308776A2, pages 73 to 87 are also described as preferable examples of the compound having an adsorptive group and a reducing group according to the invention.

These compounds can be easily synthesized by a known method in the technical field. The compound of formula (I) according to the present invention may be used alone, but it is preferred to use two or more of the compounds simultaneously. When two or more of the compounds are used, those compounds may be added to the same layer or different layers, whereby addition methods may be different from each other.

The compound represented by formula (I) according to the present invention is preferably added to the silver halide image forming layer and more preferably, the compound represented by formula (I) is added at the time of emulsion preparation. In the case where the compound is added at the time of emulsion preparation, the compound can be added at any stage in the process. For example, the compound can be added during the silver halide grain formation step; before starting of desalting step; during the desalting step; before starting of chemical ripening; during the chemical ripening step; in the step before preparing a final emulsion, or the like. The compound can be added several times during these steps. It is preferred to use the compound in the image forming layer. But the compound may be added to a protective layer or intermediate layer adjacent to the image forming layer, in combination with its addition to the image forming layer, to be diffused in the coating step.

The preferred addition amount is largely dependent on the addition method described above or the type of the compound, but is generally from 1×10⁻⁶ mol to 1 mol, preferably from 1×10⁻⁵ mol to 5×10⁻¹ mol, and more preferably from 1×10⁻⁴ mol to 1−10⁻¹ mol, per 1 mol of photosensitive silver halide.

The compound represented by formula (I) according to the present invention can be added by being dissolved in water, a water-soluble solvent such as methanol, ethanol and the like, or a mixed solution thereof. In this process, the pH may be arranged suitably by an acid or a base, and a surfactant may coexist. Further, these compounds can be added as an emulsified dispersion by dissolving them in an organic solvent having a high boiling point, and also can be added as a solid dispersion.

11) Combined Use of Silver Halides

The photosensitive silver halide emulsion in the photothermographic material according to the invention may be used alone, or two or more of them (for example, those having different mean grain sizes, different halogen compositions, different crystal habits, or different conditions for chemical sensitization) may be used together. Gradation can be controlled by using plural photosensitive silver halides each having different sensitivity. The relevant techniques include those described, for example, in JP-A Nos. 57-119341, 53-106125, 47-3929, 48-55730, 46-5187, 50-73627, and 57-150841. It is preferred to provide a sensitivity difference of 0.2 or more in terms of log E between each of the emulsions. 12) Coating Amount

The addition amount of the photosensitive silver halide, when expressed by the amount of coated silver per 1 m² of the photothermographic material, is preferably from 0.03 g/m² to 0.6 g/m², more preferably from 0.05 g/m² to 0.4 g/m², and most preferably from 0.07 g/m² to 0.3 g/m². The photosensitive silver halide is used in an amount of from 0.01 mol to 0.5 mol, preferably from 0.02 mol to 0.3 mol, and even more preferably from 0.03 mol to 0.2 mol, with respect to 1 mol of the organic silver salt.

13) Mixing Photosensitive Silver Halide and Organic Silver Salt

The mixing method and mixing conditions of the separately prepared photosensitive silver halide and organic silver salt include a method of mixing respectively prepared photosensitive silver halide grains and organic silver salt by a high speed stirrer, ball mill, sand mill, colloid mill, vibration mill, homogenizer, or the like; a method of mixing a photosensitive silver halide completed for preparation at any timing during the preparation of the organic silver salt and preparing the organic silver salt; and the like. However, so long as the effects of the invention are sufficiently realized, there is no particular restriction on the method. Further, a method of mixing two or more aqueous dispersions of organic silver salts and two or more aqueous dispersions of photosensitive silver salts while carrying out mixing is used preferably for controlling photographic properties.

14) Mixing Silver Halide into Coating Solution

In the invention, the time of adding silver halide to the coating solution for the image forming layer is preferably in a range of from 180 minutes before coating to just prior to coating, and more preferably 60 minutes before coating to 10 seconds before coating. However, so long as the effects of the invention are sufficiently realized, there is no particular restriction concerning the mixing method and the conditions of mixing. As a specific mixing method, there is a method of mixing in a tank and controlling an average residence time. The average residence time herein is calculated from addition flux and the amount of solution transferred to the coater. And another mixing method is a method using a static mixer, which is described in 8th chapter or the like of “Ekitai Kongo Gijutu” by N. Harnby, M. F. Edwards, and A. W. Nienow, translated by Koji Takahashi (Nikkan Kogyo Shinbunsha, 1989).

(Binder)

1) Binder for Image Forming Layer

Any polymer may be used as the binder for the image forming layer according to the invention. Suitable as the binder are those that are transparent or translucent, and that are generally colorless, such as natural resin or polymer and their copolymers; synthetic resin or polymer and their copolymer; or media forming a film; for example, included are gelatins, rubbers, poly(vinyl alcohols), hydroxyethyl celluloses, cellulose acetates, cellulose acetate butyrates, poly(vinyl pyrrolidones), casein, starch, poly(acrylic acids), poly(methyl methacrylates), poly(vinyl chlorides), poly(methacrylic acids), styrene-maleic anhydride copolymers, styrene-acrylonitrile copolymers, styrene-butadiene copolymers, poly(vinyl acetals) (e.g., poly(vinyl formal) and poly(vinyl butyral)), polyesters, polyurethanes, phenoxy resin, poly(vinylidene chlorides), polyepoxides, polycarbonates, poly(vinyl acetates), polyolefins, cellulose esters, and polyamides. A binder may be used with water, an organic solvent, or emulsion to form a coating solution.

In the present invention, the glass transition temperature (Tg) of the binder for the image forming layer is preferably in a range of from −20° C. to 80° C. (hereinafter, sometimes referred to as “high-Tg binder”), more preferably from 0° C. to 60° C., and even more preferably from 5° C. to 40° C.

In the specification, Tg is calculated according to the following equation:

1/Tg=Σ(Xi/Tgi)

where the polymer is obtained by copolymerization of n monomer components (from i=1 to i=n); Xi represents the weight fraction of the ith monomer (ΣXi=1), and Tgi is the glass transition temperature (absolute temperature) of the homopolymer obtained with the ith monomer. The symbol Σ stands for the summation from i=1 to i=n. Values for the glass transition temperature (Tgi) of the homopolymers derived from each of the monomers were obtained from the values of J. Brandrup and E. H. Immergut, Polymer Handbook (3rd Edition) (Wiley-Interscience, 1989).

<<Solubility Parameter>>

The solubility parameter of the binder used in the present invention is preferably in a range of from 7 (cal/cm³)^{1/2} to 15 (cal/cm³)^{1/2}, more preferably from 7.5 (cal/cm³)^{1/2} to 13 (cal/cm³)^{1/2}, and most preferably from 8 (cal/cm³)^{1/2} to 12 (cal/cm³)^{1/2}.

Calculation method of solubility parameter (SP value) is based on the method described in VII 680 to 683 of Polymer Handbook 4th edition, published by John Wiley & Sons. Solubility parameter (SP value) is a value commonly used as a factor indicating a polarity per unit volume that is expressed by cohesive energy density, namely ½ power of evaporation energy per unit volume of one molecule.

In the case of polymer, the solubility parameter is generally calculated using the following Small's equation.

SP=dΣG/M

M: Unit molecular weight of polymer

d: Density

G: A constant inherent in the atomic group or group

Solubility parameters of conventional polymer are described in VII 702 to 711 of Polymer Handbook, 4th edition, published by John Wiley & Sons. In the present invention, the value obtained by substituting Hoy's cohesive energy constant to the Small's equation mentioned above was used as the solubility parameter of the polymer.

The binder may be of two or more polymers depending on needs. And, the polymer having Tg of 20° C. or higher and the polymer having Tg of lower than 20° C. may be used in combination. In the case where two or more polymers differing in Tg are blended for use, it is preferred that the weight-average Tg is within the range mentioned above.

In the invention, the image forming layer is preferably formed by applying a coating solution containing 30% by weight or more of water in the solvent and by then drying. Therefore, it is preferred to use water-soluble or water-dispersible binder as a binder for the image forming layer.

Among the water-dispersible binder, in the case where a polymer latex having an equilibrium moisture content of 2% by weight or lower at 25° C. and 60% RH is used, the performance is enhanced.

Most preferred embodiment of the polymer latex is such prepared to yield an ion conductivity of 2.5 mS/cm or lower, and as such a preparing method, there can be mentioned a method of refining treatment using a separation function membrane after synthesizing the polymer or an ion-exchange method.

The water-soluble or water-dispersible polymer as referred herein signifies polymer which is soluble or dispersible to an aqueous solvent (water, or water containing mixed therein 70% by weight or less of a water-miscible organic solvent). Examples of the water-miscible organic solvent include alcohols such as methyl alcohol, ethyl alcohol, propyl alcohol, or the like; cellosolves such as methyl cellosolve, ethyl cellosolve, butyl cellosolve, or the like; ethyl acetate; dimethylformamide, and the like.

The term “aqueous solvent” is also used in the case where the polymer is not thermodynamically dissolved, but is present in a so-called dispersed state.

The term “equilibrium moisture content at 25° C. and 60% RH” as referred herein can be expressed as follows:

Equilibrium moisture content at 25° C. and 60% RH=[(W1−W0)/W0]×100 (% by weight)

wherein W1 is the weight of the polymer in moisture-controlled equilibrium under an atmosphere of 25° C. and 60% RH, and W0 is the absolutely dried weight at 25° C. of the polymer.

For the definition and the method of measurement for moisture content, reference can be made to Polymer Engineering Series 14, “Testing methods for polymeric materials” (The Society of Polymer Science, Japan, published by Chijin Shokan).

The equilibrium moisture content at 25° C. and 60% RH is preferably 2% by weight or lower, more preferably in a range of from 0.01% by weight to 1.5% by weight, and even more preferably from 0.02% by weight to 1% by weight.

In the invention, polymers capable of being dispersed in an aqueous solvent are particularly preferable. Examples of dispersed states may include a latex, in which water-insoluble fine particles of hydrophobic polymer are dispersed, or such in which polymer molecules are dispersed in molecular states or by forming micelles, but preferred are latex-dispersed particles. The mean particle diameter of the dispersed particles is in a range of from 1 nm to 50,000 nm, preferably from 5 nm to 1,000 nm, more preferably from 10 nm to 500 nm, and even more preferably from 50 nm to 200 nm. There is no particular limitation concerning particle diameter distribution of the dispersed particles, and the particles may be widely distributed or may exhibit a monodispersed particle diameter distribution. From the viewpoint of controlling the physical properties of the coating solution, preferred mode of usage includes mixing two or more types of dispersed particles each having monodispersed particle diameter distribution.

In the invention, preferred embodiment of the polymers capable of being dispersed in aqueous solvent includes hydrophobic polymers such as acrylic polymer, polyesters, rubbers (e.g., SBR resin), polyurethanes, poly(vinyl chlorides), poly(vinyl acetates), poly(vinylidene chlorides), polyolefins, or the like. As the polymers above, usable are straight-chain polymers, branched polymers, or crosslinked polymers; also usable are the so-called homopolymers in which one type of monomer is polymerized, or copolymers in which two or more types of monomers are polymerized. In the case of a copolymer, it may be a random copolymer or a block copolymer. The molecular weight of the polymer is, in number average molecular weight, in a range of from 5,000 to 1,000,000, and preferably from 10,000 to 200,000. Those having too small molecular weight exhibit insufficient mechanical strength on forming the image forming layer, and those having too large molecular weight are also not preferred because the resulting film-forming properties are poor. Further, crosslinking polymer latexes are particularly preferred for use.

(Specific Examples of Latex)

Specific examples of preferable polymer latex are given below, which are expressed by the starting monomers with % by weight given in parenthesis. The molecular weight is given in number average molecular weight. In the case where polyfunctional monomer is used, the concept of molecular weight is not applicable because they build a crosslinked structure. Hence, they are denoted as “crosslinking”, and the description of the molecular weight is omitted. Tg represents glass transition temperature.

P-1: Latex of -MMA(55) -EA(42) -MAA(3)—(molecular weight 39,000, Tg 39° C., SP value=9.60)

P-2: Latex of -MMA(60) -2EHA(30) -St(5) -AA(5)—(molecular weight 42,000, Tg 40° C., SP value=9.39)

P-3: Latex of -St(62) -Bu(35) -MAA(3)—(crosslinking, Tg 5° C., SP value=9.35)

P-4: Latex of -St(68) -Bu(29) -AA(3)—(crosslinking, Tg 17° C., SP value=9.38)

P-5: Latex of -St(71) -Bu(26) -AA(3)—(crosslinking, Tg 24° C., SP value=9.39)

P-6: latex of -St(70) -Bu(27) -IA(3)—(crosslinking, Tg 23° C., SP value=9.41)

P-7: Latex of -St(75) -Bu(24) -AA(])—(crosslinking, Tg 29° C., SP value=9.39)

P-8: Latex of -St(60) -Bu(35) -DVB(3) -MAA(2)—(crosslinking, Tg 6° C., SP value=9.37)

P-9: Latex of -St(70) -Bu(25) -DVB(2) -AA(3)—(crosslinking, Tg 26° C., SP value=9.41)

P-10: Latex of -VC(35) -MMA(20) -EA(35) -AN(5) -AA(5)—(molecular weight 75,000, Tg 41° C., SP value=9.92)

P-11: Latex of -VDC(65) -MMA(25) -EA(5) -MAA(5)—(molecular weight 67,000, Tg 12° C., SP value=10.04)

P-12: Latex of -EA(60) -MMA(30) -MAA(I0)—(molecular weight 12,000, Tg 16° C., SP value=9.65)

P-13: Latex of -St(70) -2EHA(27) -AA(3)—(molecular weight 130,000, Tg 43° C., SP value=9.38)

P-14: Latex of -MMA(40) -EA(58) -AA(2)—(molecular weight 43,000, Tg 18° C., SP value=9.67)

P-15: Latex of -St(70.5) -Bu(26.5) -AA(3)—(crosslinking, Tg 23° C., SP value=9.39)

P-16: Latex of -St(69.5) -Bu(27.5) -AA(3)—(crosslinking, Tg 20.5° C., SP value=9.38)

P-17: Latex of -St(61.3) -Isoprene(35.5) -AA(3)—(crosslinking, Tg 17° C., SP value=9.04)

P-18: Latex of -St(67) -Isoprene(28) -Bu(2) -AA(3)—(crosslinking, Tg 27° C., SP value=9.13)

P-19: Latex of -St(50) -Isoprene (45) -AA(5)—(crosslinking, Tg 1° C., SP value=8.96)

P-20: Latex of -St(40) -Isoprene(57) -AA(3)—(crosslinking, Tg −17° C., SP value=8.83)

P-21: Latex of -St(30) -Isoprene(67) -AA(3)—(crosslinking, Tg −30° C., SP value=8.73)

P-22: Latex of -St(70) -Isoprene(27) -AA(3)—(crosslinking, Tg 34° C., SP value=9.15)

P-23: Latex of -St(75) -Isoprene(22) -AA(3)—(crosslinking, Tg 44° C., SP value=9.20)

P-24: Latex of -St(61.3) -2,3-Dimethylbutadiene(35.5) -AA(3)—(crosslinking, Tg 17° C., SP value=9.04)

P-25: Latex of -St(61.3) -2-Chlorobutadiene(35.5) -AA(3)—(crosslinking, Tg 17° C., SP value=9.04)

In the structures above, abbreviations represent monomers as follows. MMA: methyl methacrylate, EA: ethyl acrylate, MAA: methacrylic acid, 2EHA: 2-ethylhexyl acrylate, St: styrene, Bu: butadiene, AA: acrylic acid, DVB: divinylbenzene, VC: vinyl chloride, AN: acrylonitrile, VDC: vinylidene chloride, Et: ethylene, IA: itaconic acid.

The polymer latexes described above are also commercially available, and polymers below can be utilized. Examples of acrylic polymer include Cevian A-4635, 4718, and 4601 (all manufactured by Daicel Chemical Industries, Ltd.), Nipol Lx811, 814, 821, 820, and 857 (all manufactured by Nippon Zeon Co., Ltd.), and the like; examples of polyesters include FINETEX ES650, 611, 675, and 850 (all manufactured by Daimppon Ink and Chemicals, Inc.), WD-size and WMS (all manufactured by Eastman Chemical Co.), and the like; examples of polyurethanes include HYDRAN AP10, 20, 30, and 40 (all manufactured by Dainippon Ink and Chemicals, Inc.), and the like; examples of rubbers include LACSTAR 7310K, 3307B, 4700H, and 7132C (all manufactured by Dainippon Ink and Chemicals, Inc.), Nipol Lx416, 410, 438C, and 2507 (all manufactured by Nippon Zeon Co., Ltd.), and the like; examples of poly(vinyl chlorides) include G351 and G576 (all manufactured by Nippon Zeon Co., Ltd.), and the like; examples of poly(vinylidene chlorides) include L502 and L513 (all manufactured by Asahi Chemical Industry Co., Ltd.), and the like; and examples of polyolefins include Chemipearl S120 and SA100 (all manufactured by Mitsui Petrochemical Industries, Ltd.), and the like.

The polymer latex above may be used alone, or may be used by blending two or more of them depending on needs.

(Preferable Latex)

Particularly preferable as the polymer latex for use in the invention is that of styrene-butadiene copolymer or that of styrene-isoprene copolymer. The weight ratio of the monomer unit of styrene relative to that of butadiene in the styrene-butadiene copolymer is preferably in a range of from 40:60 to 95:5. Further, the monomer unit of styrene and that of butadiene preferably account for 60% by weight to 99% by weight with respect to the copolymer. Further, the polymer latex according to the invention preferably contains acrylic acid or methacrylic acid in a range of from 1% by weight to 6% by weight with respect to the sum of styrene and butadiene, and more preferably from 2% by weight to 5% by weight. The polymer latex according to the invention preferably contains acrylic acid. Preferable range of monomer content is similar to that described above. Further, the ratio of copolymerization and the like in the styrene-isoprene copolymer are the same as those in the case of styrene-butadiene copolymer.

As the latex of styrene-butadiene copolymer preferably used in the invention, there are mentioned P-3 to P-9, P-15, and P-16 described above, and commercially available LACSTAR-3307B, 7132C, Nipol Lx416, and the like. And as preferred examples of the latex of styrene-isoprene copolymer, there are mentioned P-17 to P-23 described above.

In the image forming layer of the photothermographic material according to the invention, if necessary, there may be added hydrophilic polymer such as gelatin, poly(vinyl alcohol), methyl cellulose, hydroxypropyl cellulose, carboxymethyl cellulose, or the like. The hydrophilic polymer is preferably added in an amount of 30% by weight or less, and more preferably 20% by weight or less, with respect to the total weight of the binder incorporated in the image forming layer.

According to the invention, the layer containing organic silver salt (namely, the image forming layer) is preferably formed by using polymer latex. Concerning the amount of the binder for the image forming layer, the weight ratio of entire binder to organic silver salt is preferably in a range of from 1/10 to 10/1, more preferably from 1/3 to 5/1, and even more preferably from 1/1 to 3/1.

The layer containing organic silver salt is, in general, a photosensitive layer (image forming layer) containing a photosensitive silver halide, i.e., the photosensitive silver salt; and in such a case, the weight ratio of entire binder to silver halide is in a range of from 5 to 400, and more preferably from 10 to 200.

The total amount of binder in the image forming layer according to the invention is preferably in a range of from 0.2 g/m² to 30 g/m², more preferably from 1 g/m² to 15 g/m², and even more preferably from 2 g/m² to 10 g/m². To the image forming layer according to the invention, there may be added a crosslinking agent for crosslinking, a surfactant to improve coating ability, or the like.

2) Binder for Non-Photosensitive Layer

For the binder which can be used in the non-photosensitive layer of the photothermographic material according to the present invention, the same binder as the binder which can be used in the image forming layer can be used. But as the main binder for the non-photosensitive layer, it is preferable to use a water-soluble binder. The main binder means a binder which occupies 50% or more, preferably 60% or more, of the binder in the non-photosensitive layer.

Examples of the water-soluble main binder which can be used in the non-photosensitive layer include gelatin, poly(vinyl alcohol), methyl cellulose, hydroxypropyl cellulose, carboxymethyl cellulose, and the like. Particularly, gelatin or poly(vinyl alcohol) is preferable.

(Preferred Solvent of Coating Solution)

In the invention, a solvent of a coating solution for the image forming layer of the photothermographic material (wherein a solvent and dispersion medium are collectively represented as a solvent for simplicity) is preferably an aqueous solvent containing water at 30% by weight or more. Examples of components other than water may include any of water-miscible organic solvents such as methyl alcohol, ethyl alcohol, isopropyl alcohol, methyl cellosolve, ethyl cellosolve, dimethylformamide and ethyl acetate. The content of water in a solvent of the coating solution is more preferably 50% by weight or higher, and even more preferably 70% by weight or higher. Examples of a preferable solvent composition include, in addition to water, water/methyl alcohol=90/10, water/methyl alcohol=70/30, water/methyl alcohol/dimethylformamide=80/15/5, water/methyl alcohol/ethyl cellosolve=85/10/5, water/methyl alcohol/isopropyl alcohol=85/10/5, and the like (wherein the numerals are values in % by weight).

(Antifoggant)

As an antifoggant, stabilizer, and stabilizer precursor which can be used in the invention, there are mentioned those of patents described in paragraph No. 0070 of JP-A No. 10-62899 and in line 57 of page 20 to line 7 of page 21 of EP-A No. 0803764A1, the compounds described in JP-A Nos. 9-281637 and 9-329864, and the compounds described in U.S. Pat. No. 6,083,681, and EP-A No. 1048975.

1) Organic Polyhalogen Compound

Preferable organic polyhalogen compound that can be used in the invention is explained specifically below. In the invention, preferred organic polyhalogen compound is a compound represented by the following formula (H).

Q-(Y)n—C(Z₁)(Z₂)X Formula (H)

In formula (H), Q represents an alkyl group, an aryl group, or a heterocyclic group; Y represents a divalent linking group; n represents 0 or 1; Z₁ and Z₂ each represent a halogen atom; and X represents a hydrogen atom or an electron-attracting group.

In formula (H), Q is preferably an alkyl group having 1 to 6 carbon atoms, an aryl group having 6 to 12 carbon atoms, or a heterocyclic group comprising at least one nitrogen atom (pyridine, quinoline, or the like).

In the case where Q is an aryl group in formula (H), Q is preferably a phenyl group substituted by an electron-attracting group whose Hammett substituent constant σp yields a positive value. For the details of Hammett substituent constant, reference can be made to Journal of Medicinal Chemistry, vol. 16, No. 11 (1973), pages 1207 to 1216, and the like. Examples of the electron-attracting group include a halogen atom, an alkyl group substituted by an electron-attracting group, an aryl group substituted by an electron-attracting group, a heterocyclic group, an alkylsulfonyl group, an arylsulfonyl group, an acyl group, an alkoxycarbonyl group, a carbamoyl group, a sulfamoyl group, and the like. Preferable as the electron-attracting group is a halogen atom, a carbamoyl group, or an arylsulfonyl group, and a carbamoyl group is particularly preferable.

X is preferably an electron-attracting group. As the electron-attracting group, preferable are a halogen atom, an aliphatic arylsulfonyl group, a heterocyclic sulfonyl group, an alkylsulfonyl group, an aliphatic arylacyl group, a heterocyclic acyl group, an aliphatic aryloxycarbonyl group, a heterocyclic oxycarbonyl group, a carbamoyl group, and a sulfamoyl group; more preferable are a halogen atom and a carbamoyl group; and particularly preferable is a bromine atom.

Z₁ and Z₂ each are preferably a bromine atom or an iodine atom, and more preferably, a bromine atom.

Y preferably represents —C(═O)—, —SO—, —SO₂—, —C(═O)N(R)—, or —SO₂N(R)—; more preferably, —C(═O)—, —SO₂—, or —C(═O)N(R)—; and particularly preferably, —SO₂— or —C(═O)N(R)—. Herein, R represents a hydrogen atom, an aryl group, or an alkyl group. R is preferably a hydrogen atom or an alkyl group, and particularly preferably a hydrogen atom.

n represents 0 or 1, and is preferably 1.

In formula (H), in the case where Q is an alkyl group, Y is preferably —C(═O)N(R)—. And, in the case where Q is an aryl group or a heterocyclic group, Y is preferably —SO₂—.

In formula (H), the embodiment where the residues, which are obtained by removing a hydrogen atom from the compound, bond to each other (generally called bis type, tris type, or tetrakis type) is also preferably used.

In formula (H′), the embodiment having, as a substituent, a dissociative group (for example, a COOH group or a salt thereof, an SO₃H group or a salt thereof, a PO₃H group or a salt thereof, or the like), a group containing a quaternary nitrogen cation (for example, an ammonio group, a pyridinio group, or the like), a polyethyleneoxy group, a hydroxy group, or the like is also preferable.

Specific examples of the compound represented by formula (H) according to the invention are shown below.

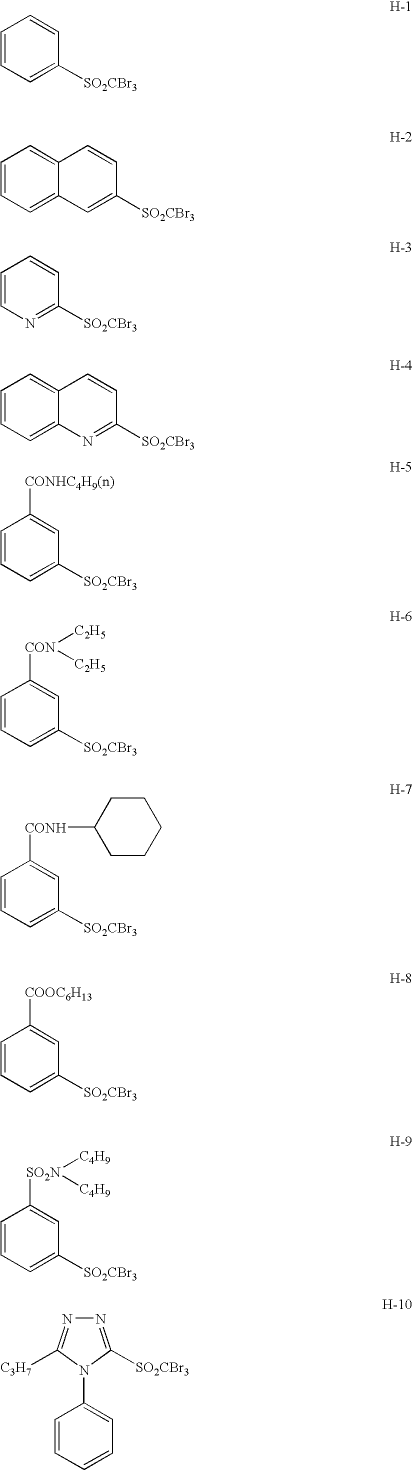

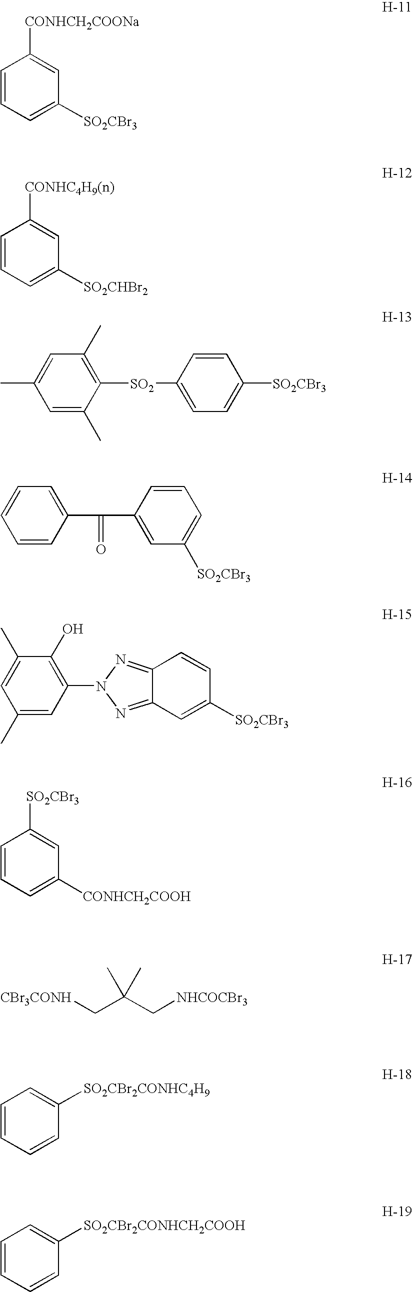

As preferred organic polyhalogen compounds which can be used in the present invention other than those above, there are mentioned compounds described as illustrated compounds of the relevant invention in the specifications of U.S. Pat. Nos. 3,874,946, 4,756,999, 5,340,712, 5,369,000, 5,464,737, and 6,506,548, and JP-A Nos. 50-137126, 50-89020, 50-119624, 59-57234, 7-2781, 7-5621, 9-160164, 9-244177, 9-244178, 9-160167, 9-258367, 9-265150, 9-319022, 10-197988, 10-197989, 11-242304, 2000-2963, 2000-112070, 2000-284410, 2000-284412, 2001-33911, 2001-31644, 2001-312027, and 2003-50441. Particularly, the compounds specifically illustrated in JP-A Nos. 7-2781, 2001-33911, and 2001-312027 are preferable.

The compound represented by formula (H) according to the invention is preferably used in an amount of from 10⁻⁴ mol to 1 mol, more preferably from 10⁻³ mol to 0.5 mol, and even more preferably from 1×10⁻² mol to 0.2 mol, per 1 mol of non-photosensitive silver salt incorporated in the image forming layer.

In the invention, methods which can be used for incorporating the antifoggant into the photothermographic material are those described above in the method for incorporating the reducing agent, and also for the organic polyhalogen compound, it is preferably added in the form of a solid fine particle dispersion.

2) Other Antifoggants

As other antifoggants, there are mentioned a mercury (II) salt described in paragraph number 0113 of JP-A No. 11-65021, benzoic acids described in paragraph number 0114 of the same literature, a salicylic acid derivative described in JP-A No. 2000-206642, a formalin scavenger compound represented by formula (S) in JP-A No. 2000-221634, a triazine compound related to claim 9 of JP-A No. 11-352624, a compound represented by formula (III), 4-hydroxy-6-methyl-1,3,3a,7-tetrazaindene, described in JP-A No. 6-11791, and the like.

The photothermographic material according to the invention may further contain an azolium salt in order to prevent fogging. Azolium salts useful in the present invention include a compound represented by formula (XI) described in JP-A No. 59-193447, a compound described in JP-B No. 55-12581, and a compound represented by formula (II) described in JP-A No. 60-153039. The azolium salt may be added to any part of the photothermographic material, but as the layer to be added, it is preferred to select a layer on the side having the image forming layer, and more preferred is to select the image forming layer itself. The azolium salt may be added at any time of the process of preparing the coating solution; in the case where the azolium salt is added into the image forming layer, any time of the process may be selected from the preparation of the organic silver salt to the preparation of the coating solution, but preferred is to add the azolium salt within a period after preparation of the organic silver salt until just prior to coating. As the method for adding the azolium salt, any method such as in the form of powder, a solution, a fine particle dispersion, or the like may be used. Furthermore, the azolium salt may be added as a solution having mixed therein other additives such as a sensitizing agent, reducing agent, toner, or the like. In the invention, the azolium salt may be added in any amount, but preferably, it is added in an amount of from 1×10⁻⁶ mol to 2 mol, and more preferably from 1×10⁻³ mol to 0.5 mol, per 1 mol of silver.

(Other Additives)

1) Mercapto Compounds, Disulfides, and Thiones

In the invention, mercapto compounds, disulfide compounds, and thione compounds can be added in order to control the development by suppressing or enhancing development, to improve spectral sensitization efficiency, and to improve storability before development and storability after development. Descriptions can be found in paragraph numbers 0067 to 0069 of JP-A No. 10-62899, as compounds represented by formula (I) and specific examples thereof shown in paragraph numbers 0033 to 0052 of JP-A No. 10-186572, and in lines 36 to 56 in page 20 of EP No. 0803764A1. Among them, mercapto-substituted heterocyclic aromatic compounds described in JP-A Nos. 9-297367, 9-304875, 2001-100358, 2002-303954, 2002-303951, and the like are preferred.

2) Plasticizer and Lubricant

In the invention, well-known plasticizer and lubricant can be used to improve physical properties of film. Particularly, to improve handling facility during manufacturing process or resistance to scratch during thermal development, it is preferred to use a lubricant such as liquid paraffin, a long chain fatty acid, an amide of a fatty acid, an ester of a fatty acid, or the like. Particularly preferred are liquid paraffin, in which components having a low boiling point are removed, and an ester of a fatty acid which has a branched structure and a molecular weight of 1000 or more.

Concerning plasticizers and lubricants which can be used in the image forming layer and non-photosensitive layer, compounds described in paragraph No. 0117 of JP-A No. 11-65021 and in JP-A Nos. 2000-5137, 2004-219794, 2004-219802, and 2004-334077 are preferable.

3) Dyes, Pigments, and Dye Fixing Agent

From the viewpoints of improving color tone, preventing the generation of interference fringes upon laser exposure, preventing irradiation, and preventing halation, various dyes and pigments can be used in the image forming layer and non-photosensitive layer according to the invention. As the dye, a metal phthalocyanine dye (dyes described in JP-A Nos. 2003-295388 and the like) can be used preferably.

4) Nucleation Accelerator

In the photothermographic material according to the invention, it is preferred to use in combination an acid obtained by hydration of diphosphorus pentaoxide or a salt thereof as a nucleation accelerator. Acids obtained by hydration of diphosphorus pentaoxide or salts thereof include metaphosphoric acid (salt), pyrophosphoric acid (salt), orthophosphoric acid (salt), triphosphoric acid (salt), tetraphosphoric acid (salt), hexametaphosphoric acid (salt), and the like. Particularly preferred acids obtained by hydration of diphosphorus pentaoxide or salts thereof include orthophosphoric acid (salt) and hexametaphosphoric acid (salt). Specific examples of the salt include sodium orthophosphate, sodium dihydrogen orthophosphate, sodium hexametaphosphate, ammonium hexametaphosphate, and the like.

The addition amount of the acid obtained by hydration of diphosphorus pentaoxide or the salt thereof (i.e., the coating amount per 1 m² of the photothermographic material) may be set as desired depending on sensitivity or fogging, but the addition amount is preferably from 0.1 mg/m² to 500 mg/m², and more preferably from 0.5 mg/m² to 100 mg/m².

(Preparation of Coating Solution and Coating)

The temperature for preparing the coating solution for the image forming layer according to the invention is preferably from 30° C. to 65° C., more preferably 35° C. or higher and lower than 60° C., and even more preferably from 35° C. to 55° C. Furthermore, the temperature of the coating solution for the image forming layer immediately after adding the polymer latex is preferably maintained within the temperature range of from 30° C. to 65° C.

(Layer Constitution and Constituent Components)

The photothermographic material according to the invention has one or more image forming layers constructed on a support. In the case of constituting the image forming layer from one layer, the image forming layer comprises an organic silver salt, a photosensitive silver halide, a reducing agent, and a binder, and may further comprise additional materials as desired and necessary, such as a toner, a film-forming promoting agent, and other auxiliary agents. In the case of constituting the image forming layer from two or more layers, the first image forming layer (in general, a layer placed nearer to the support) contains an organic silver salt and a photosensitive silver halide. Some of the other components are incorporated in the second image forming layer or in both of the layers. The constitution of a multicolor photothermographic material may include combinations of two layers for those for each of the colors, or may contain all the components in a single layer such as described in U.S. Pat. No. 4,708,928. In the case of multi-dye multicolor photosensitive photothermographic material, each of the image forming layers is maintained distinguished from each other by incorporating functional or non-functional barrier layer between each of the image forming layers such as described in U.S. Pat. No. 4,460,681.

The photothermographic material according to the invention can have a non-photosensitive layer in addition to the image forming layer. Non-photosensitive layers can be classified depending on the layer arrangement into (a) a surface protective layer provided on the image forming layer (on the side farther from the support), (b) an intermediate layer provided among plural image forming layers or between the image forming layer and the protective layer, (c) an undercoat layer provided between the image forming layer and the support, and (d) a back layer which is provided on the opposite side of the support from the image forming layer.

Furthermore, a layer that functions as an optical filter may be provided as (a) or (b) above. An antihalation layer may be provided as (c) or (d) to the photothermographic material.

1) Surface Protective Layer

The photothermographic material according to the invention can comprise a surface protective layer with an object to prevent adhesion of the image forming layer, or the like. The surface protective layer may be a single layer or plural layers.

Description on the surface protective layer may be found in paragraph Nos. 0119 and 0120 of JP-A No. 11-65021 and in JP-A No. 2000-171936.

Preferable binder of the surface protective layer according to the invention is gelatin, but poly(vinyl alcohol) (PVA) is also preferably used instead, or in combination. As gelatin, there can be used inert gelatin (e.g., Nitta gelatin 750), phthalated gelatin (e.g., Nitta gelatin 801), and the like. Usable as PVA are those described in paragraph Nos. 0009 to 0020 of JP-A No. 2000-171936, and preferred are the completely saponified product PVA-105, the partially saponified product PVA-205 and PVA-335, as well as modified poly(vinyl alcohol) MP-203 (all trade names of products from Kuraray Ltd.), and the like. The amount of coated poly(vinyl alcohol) (per 1 m² of support) in the protective layer (per one layer) is preferably in a range of from 0.3g/m² to 4.0 g/m², and more preferably from 0.3 g/m² to 2.0 g/m².

The total amount of the coated binder (including water-soluble polymer and latex polymer) (per 1 m² of support) in the surface protective layer (per one layer) is preferably in a range of from 0.3 g/m² to 5.0 g/m², and more preferably from 0.3 g/m² to 2.0 g/m².

Further, it is preferable to use a lubricant such as liquid paraffin, an ester of a fatty acid, or the like in the surface protective layer. The addition amount of the lubricant is in a range of from 1 mg/m² to 200 mg/m², preferably from 10 mg/m² to 150 mg/m², and more preferably from 20 mg/m² to 100 mg/m².

2) Antihalation Layer

The photothermographic material according to the present invention can comprise an antihalation layer provided to the side farther from the light source than the image forming layer.

Descriptions on the antihalation layer can be found in paragraph Nos. 0123 and 0124 of JP-A No. 11-65021, in JP-A Nos. 11-223898, 9-230531, 10-36695, 10-104779, 11-231457, 11-352625, 11-352626, and the like.

The antihalation layer contains an antihalation dye having its absorption at the wavelength of the exposure light. In the case where the exposure wavelength is in the infrared region, it is enough that an infrared-absorbing dye is used, and in such a case, preferred are dyes having no absorption in the visible light region.

In the case of preventing halation from occurring by using a dye having absorption in the visible light region, it is preferred that the color of the dye would not substantially reside after image formation or it is preferred to use a dye having low visibility.

Concerning the method for decoloring the dye after image formation, it is preferred to employ a means for decoloring by the heat of thermal development; in particular, it is preferred to add a thermal bleaching dye and a base precursor to a non-photosensitive layer to impart function as an antihalation layer. Those techniques are described in JP-A No. 11-231457 and the like.

Further, the use of a dye having sharp absorption in the exposure wavelength makes it possible to obtain low visibility and sufficient antihalation effect. By using such dye, it is not necessary to decolor the dye after image formation. Those techniques are described in JP-A No. 2003-262934.

The addition amount of the antihalation dye is determined depending on the usage of the dye. In general, it is used at an amount as such that the optical density (absorbance) exceeds 0.1 when measured at the desired wavelength. The optical density is preferably in a range of from 0.15 to 2, and more preferably from 0.2 to 1. The addition amount of the dye to obtain optical density in the above range is generally from 0.001 g/m² to 1 g/m².

Two or more dyes that are different from each other may be used.

In the method for decoloring the dye, similarly, two or more base precursors may be used in combination.

In the case of thermal decolorization by the combined use of a bleaching dye and a base precursor, it is preferable from the viewpoints of thermal decoloring property or the like to further use a substance (e.g., diphenylsulfone, 4-chlorophenyl(phenyl)sulfone, 2-naphthylbenzoate, or the like) which lowers the melting point by at least 3° C. when mixed with the base precursor, such as described in JP-A No. 11-352626.

The dye described above is also preferably used in the image forming layer for the purpose of preventing irradiation.

3) Back Layer

Back layers that can be used in the invention are described in paragraph Nos. 0128 to 0130 of JP-A No. 11-65021.

In the invention, coloring matters having maximum absorption in the wavelength range of from 300 nm to 450 nm can be added in order to improve color tone of developed silver images and deterioration of the images during aging. Such coloring matters are described in, for example, JP-A Nos. 62-210458, 63-104046, 63-103235, 63-208846, 63-306436, 63-314535, 01-61745, 2001-100363, and the like.

Such coloring matters are generally added in a range of from 0.1 mg/m² to 1 g/m², preferably to the back layer which is provided to the opposite side of the support from the image forming layer.

Further, in order to control the base color tone, it is preferred to use a dye having an absorption peak in a wavelength range from 580 nm to 680 nm. As a dye satisfying this purpose, preferred are oil-soluble azomethine dyes described in JP-A Nos. 4-359967 and 4-359968, or water-soluble phthalocyanine dyes described in JP-A No. 2003-295388, which have low absorption intensity on the short wavelength side. The dyes for this purpose may be added to any of the layers, but more preferred is to add them in the image forming layer or non-photosensitive layer on the image forming layer side, or on the backside.

The photothermographic material according to the invention is preferably a so-called one-side photosensitive material, which comprises at least one image forming layer containing silver halide emulsion on one side of the support, and a back layer on the other side.

Concerning the layer constitution of the back layer, it is constituted of two layers including a layer containing a dye and a protective layer, but such as described in JP-A No. 2006-189508, it is preferred to dispose further an undercoat layer between the layer containing a dye and the support in order to improve coating ability. It is preferred that these two or three layers are simultaneously coated and dried.

4) Matting Agent

A matting agent is preferably added to the photothermographic material according to the invention in order to improve transportability. Description on the matting agent can be found in paragraphs Nos. 0126 and 0127 of JP-A No. 11-65021. The addition amount of the matting agent is preferably in a range of from 1 mg/m² to 400 mg/m², and more preferably from 5 mg/M² to 300 mg/m², with respect to the coating amount per 1 m² of the photothermographic material.

In the invention, the shape of the matting agent may be a fixed form or non-fixed form, but preferred is to use those having a fixed form and a spherical shape.

The volume-weighted mean equivalent spherical diameter of the matting agent used in the image forming layer surface is preferably in a range of from 0.3 μm to 10 μm, and more preferably from 0.5 μm to 7 μm. Further, the particle distribution of the matting agent is preferably set as such that the variation coefficient is from 5% to 80%, and more preferably from 20% to 80%. Herein, the variation coefficient is defined by (the standard deviation of particle diameter)/(mean diameter of the particle)×100. Furthermore, two or more types of matting agents having different mean particle size can be used in the image forming layer surface. In this case, the difference between the mean particle size of the biggest matting agent and the mean particle size of the smallest matting agent is preferably from 2 μm to 8 μm, and more preferably from 2 μm to 6 μm.

Volume weighted mean equivalent spherical diameter of the matting agent used in the back surface is preferably in a range of from 1 μm to 15 μm, and more preferably from 3 μm to 10 μm. Further, the particle distribution of the matting agent is preferably set as such that the variation coefficient is from 3% to 50%, and more preferably from 5% to 30%. Furthermore, two or more types of matting agents having different mean particle size can be used in the back surface. In this case, the difference between the mean particle size of the biggest matting agent and the mean particle size of the smallest matting agent is preferably from 2 μm to 14 μm, and more preferably from 2 μm to 9 μm.

The level of matting on the image forming layer surface is not restricted as long as star-dust trouble does not occur, but the level of matting is preferably from 30 sec to 2000 sec, and particularly preferably from 40 sec to 1500 sec, when expressed by a Beck's smoothness. Beck's smoothness can be calculated easily, using Japan Industrial Standard (JIS) P8119 “The method of testing Beck's smoothness for papers and sheets using a Beck's test apparatus”, or TAPPI standard method T479.

In the invention, the level of matting of the back layer is preferably in a range of 1200 sec or less and 10 sec or more; more preferably, 800 sec or less and 20 sec or more; and even more preferably, 500 sec or less and 40 sec or more, when expressed by a Beck's smoothness.

In the present invention, a matting agent is preferably contained in an outermost layer of the photothermographic material, in a layer which functions as an outermost layer, or in a layer nearer to outer surface, and is also preferably contained in a layer which functions as a so-called protective layer.

5) Polymer Latex

In the present invention, polymer latex is preferably used in the surface protective layer or the back layer of the photothermographic material. As such polymer latex, descriptions can be found in “Gosei Jushi Emulsion (Synthetic resin emulsion)” (Taira Okuda and Hiroshi Inagaki, Eds., published by Kobunshi Kankokai (1978)), “Gosei Latex no Oyo (Application of synthetic latex)” (Takaaki Sugimura, Yasuo Kataoka, Soichi Suzuki, and Keiji Kasahara, Eds., published by Kobunshi Kankokai (1993)), and “Gosei Latex no Kagaku (Chemistry of synthetic latex)” (Soichi Muroi, published by Kobunshi Kankokai (1970)), and the like. More specifically, there are mentioned a latex of methyl methacrylate (33.5% by weight)/ethyl acrylate (50% by weight)/methacrylic acid (16.5% by weight) copolymer, a latex of methyl methacrylate (47.5% by weight)/butadiene (47.5% by weight)/itaconic acid (5% by weight) copolymer, a latex of ethyl acrylate/methacrylic acid copolymer, a latex of methyl methacrylate (58.9% by weight)/2-ethylhexyl acrylate (25.4% by weight)/styrene (8.6% by weight)/2-hydroxyethyl methacrylate (5.1% by weight)/acrylic acid (2.0% by weight) copolymer, a latex of methyl methacrylate (64.0% by weight)/styrene (9.0% by weight)/butyl acrylate (20.0% by weight)/2-hydroxyethyl methacrylate (5.0% by weight)/acrylic acid (2.0% by weight) copolymer, and the like. Furthermore, as the binder for the surface protective layer, there may be applied the technology described in paragraph Nos. 0021 to 0025 of the specification of JP-A No. 2000-267226, and the technology described in paragraph Nos. 0023 to 0041 of the specification of JP-A No. 2000-19678. The polymer latex in the surface protective layer is preferably contained in an amount of from 10% by weight to 90% by weight, particularly preferably from 20% by weight to 80% by weight, with respect to the total weight of binder.

6) Film Surface pH

The film surface pH of the photothermographic material according to the invention preferably yields a pH of 7.0 or lower, and more preferably 6.6 or lower, before thermal developing processing. Although there is no particular restriction concerning the lower limit, the lower limit of pH value is about 3. The most preferred film surface pH range is from 4 to 6.2. From the viewpoint of reducing the film surface pH, it is preferred to use an organic acid such as a phthalic acid derivative or a non-volatile acid such as sulfuric acid, or a volatile base such as ammonia for the adjustment of the film surface pH. In particular, ammonia is preferably used for the achievement of low film surface pH, because it can easily vaporize to remove it in the coating step or before applying thermal development.

It is also preferred to use a non-volatile base such as sodium hydroxide, potassium hydroxide, lithium hydroxide, or the like, in combination with ammonia. The method of measuring the film surface pH value is described in paragraph No. 0123 of the specification of JP-A No. 2000-284399.

7) Hardener

A hardener may be used in each of the image forming layer, protective layer, back layer, and the like according to the invention.

As examples of the hardener, descriptions of various methods can be found in pages 77 to 87 of T. H. James, “THE THEORY OF THE PHOTOGRAPHIC PROCESS, FOURTH EDITION” (Macmillan Publishing Co., Inc., 1977). Preferably used are, in addition to chromium alum, sodium salt of 2,4-dichloro-6-hydroxy-s-triazine, N,N-ethylenebis(vinylsulfonacetamide), and N,N-propylenebis(vinylsulfonacetamide), polyvalent metal ions described in page 78 of the above literature and the like, polyisocyanates described in U.S. Pat. No. 4,281,060, JP-A No. 6-208193, and the like, epoxy compounds of U.S. Pat. No. 4,791,042 and the like, and vinylsulfone compounds of JP-A No. 62-89048 and the like.

The hardener is added as a solution, and this solution is added to the coating solution for the protective layer within a period from 180 minutes before coating to just before coating, and preferably within a period from 60 minutes before coating to 10 seconds before coating. However, so long as the effects of the invention are sufficiently realized, there is no particular restriction concerning the mixing method and the conditions of mixing. As specific mixing methods, there can be mentioned a method of mixing in the tank, in which the average stay time calculated from the flow rate of addition and the feed rate to the coater is controlled to yield a desired time, and a method using static mixer such as described in Chapter 8 of N. Harnby, M. F. Edwards, and A. W. Nienow (translated by Koji Takahashi) “Ekitai Kongo Gijutu (Liquid Mixing Technology)” (Nikkan Kogyo Shinbunsha, 1989), and the like.

8) Surfactant

Concerning the surfactant, the solvent, the support, the antistatic or electrically conductive layer, and the method for obtaining color images applicable in the invention, there can be used those described in paragraph numbers 0132, 0133, 0134, 0135, and 0136, respectively, of JP-A No. 11-65021. Concerning lubricants, there can be used those described in paragraph numbers 0061 to 0064 of JP-A No. 11-84573 and in paragraph numbers 0049 to 0062 of JP-A No. 2001-83679.

In the invention, it is preferred to use a fluorocarbon surfactant. Specific examples of the fluorocarbon surfactant include the compounds described in JP-A Nos. 10-197985, 2000-19680, and 2000-214554. Polymer fluorocarbon surfactants described in JP-A No. 9-281636 are also used preferably. For the photothermographic material according to the invention, the fluorocarbon surfactants described in JP-A Nos. 2002-82411, 2003-57780, and 2003-149766 are preferably used. In the case of conducting coating manufacture with an aqueous coating solution, the usage of the fluorocarbon surfactants described in JP-A Nos. 2003-57780 and 2001-264110 is particularly preferred viewed from the standpoints of capacity in static control, stability of the coated surface state, and sliding capability. The fluorocarbon surfactant described in JP-A No. 2001-264110 is most preferred because of high capacity in static control and that it needs small amount to use.

According to the invention, the fluorocarbon surfactant can be used on either side of the image forming layer side or the backside, but is preferably used on the two sides. Further, it is particularly preferred to use it in combination with an electrically conductive layer including metal oxides described below. In this case, sufficient performance is obtained even if the amount of the fluorocarbon surfactant on the side having the electrically conductive layer is reduced or removed.

The addition amount of the fluorocarbon surfactant is preferably in a range of from 0.1 mg/m² to 100 mg/m² on each side of the image forming layer side and backside, more preferably from 0.3 mg/m² to 30 mg/m², and even more preferably from 1 mg/m² to 10 mg/m². Especially, the fluorocarbon surfactant described in JP-A No. 2001-264110 is effective, and is preferably used in a range of from 0.01 mg/m² to 10 mg/m², and more preferably in a range of from 0.1 mg/m² to 5 mg/m².

9) Antistatic Agent

The photothermographic material according to the invention preferably has an antistatic layer including metal oxides or electrically conductive polymer. The antistatic layer may serve as an undercoat layer, a back surface protective layer, or the like, but can also be placed specially. As an electrically conductive material included in the antistatic layer, metal oxides having enhanced electric conductivity by the method of introducing oxygen defects or different types of metallic atoms into the metal oxides are preferable for use. Preferred examples of the metal oxide include ZnO, TiO₂, and SnO₂. The addition of Al, or In with respect to ZnO, the addition of Sb, Nb, P, halogen element, or the like with respect to SnO₂, and the addition of Nb, Ta, or the like with respect to TiO₂ are preferred. Particularly preferred for use is SnO₂ combined with Sb. The addition amount of heteroatom is preferably in a range of from 0.01 mol % to 30 mol %, and more preferably in a range of from 0.1 mol % to 10 mol %. The shape of the metal oxide includes, for example, spherical, needle-like, or tabular shape. Needle-like particle, in which a ratio of (the major axis)/(the minor axis) is 2.0 or higher, and more preferably from 3.0 to 50, is preferred viewed from the standpoint of the electric conductivity effect. The metal oxide is preferably used in a range of from 1 mg/m² to 1000 mg/m², more preferably from 10 mg/m² to 500 mg/m², and even more preferably from 20 mg/m² to 200 mg/m². The antistatic layer according to the invention may be disposed on either side of the image forming layer side or the backside, but it is preferred to set between the support and the back layer. Specific examples of the antistatic layer according to the invention are described in paragraph Nos. 0135 of JP-A No. 11-65021, in JP-A Nos. 56-143430, 56-143431, 58-62646, and 56-120519, and in paragraph Nos. 0040 to 0051 of JP-A No. 11-84573, in U.S. Pat. No. 5,575,957, and in paragraph Nos. 0078 to 0084 of JP-A No. 11-223898.

10) Support

As the transparent support, preferably used is polyester, particularly, polyethylene terephthalate, which is subjected to heat treatment in the temperature range of from 130° C. to 185° C. in order to relax the internal strain which is caused by biaxial stretching and remaining inside the film, and to remove strain ascribed to heat shrinkage generated during thermal development. In the case of a photothermographic material for medical use, the transparent support may be colored with a blue dye (for instance, dye-1 described in the Example of JP-A No. 8-240877), or may be uncolored. Concerning the support, it is preferred to apply undercoating technology such as water-soluble polyester described in JP-A No. 11-84574, a styrene-butadiene copolymer described in JP-A No. 10-186565, a vinylidene chloride copolymer described in JP-A No. 2000-39684, or the like. The moisture content of the support is preferably 0.5% by weight or lower, when coating for image forming layer or back layer is conducted on the support.

11) Other Additives

Furthermore, an antioxidant, stabilizer, plasticizer, ultraviolet absorber, or film-forming promoting agent may be added to the photothermographic material according to the invention. Each of the additives is added to either of the image forming layer or the non-photosensitive layer. Reference can be made to WO No. 98/36322, EP No. 803764A1, JP-A Nos. 10-186567 and 10-18568, and the like.

12) Coating Method

The photothermographic material according to the invention may be coated by any method. Specifically, various types of coating operations including extrusion coating, slide coating, curtain coating, immersion coating, knife coating, flow coating, or an extrusion coating using the type of hopper described in U.S. Pat. No. 2,681,294 are used. Preferably used is slide coating or extrusion coating described in pages 399 to 536 of Stephen F. Kistler and Petert M. Schweizer, “LIQUID FILM COATING” (Chapman & Hall, 1997), and particularly preferably used is slide coating. An example of the shape of the slide coater for use in slide coating is shown in FIG. 11b.1, page 427, of the same literature. If desired, two or more layers can be coated simultaneously by the method described in pages 399 to 536 of the same literature or by the method described in U.S. Pat. No. 2,761,791 and British Patent No. 837,095. Particularly preferable coating method in the invention is the method described in JP-A Nos. 2001-194748, 2002-153808, 2002-153803, and 2002-182333.

The coating solution for the image forming layer according to the invention is preferably a so-called thixotropic fluid. For the details of this technology, reference can be made to JP-A No. 11-52509. Viscosity of the coating solution for the image forming layer according to the invention at a shear velocity of 0.1 S⁻¹ is preferably from 400 mPa·s to 100,000 mPa·s, and more preferably from 500 mPa·s to 20,000 mPa·s. At a shear velocity of 1000 S⁻¹, the viscosity is preferably from 1 mPa·s to 200 mPa·s, and more preferably from 5 mPa·s to 80 mPa·s.

In the case of mixing two types of liquids on preparing the coating solution used for the invention, known in-line mixer or in-plant mixer is preferably used. Preferred in-line mixer used for the invention is described in JP-A No. 2002-85948, and preferred in-plant mixer used for the invention is described in JP-A No. 2002-90940.

The coating solution according to the invention is preferably subjected to antifoaming treatment to maintain the coated surface in a good state. Preferred method for antifoaming treatment in the invention is described in JP-A No. 2002-66431.

In the case of applying the coating solution according to the invention to the support, it is preferred to perform diselectrification in order to prevent adhesion of dust, particulates, and the like due to charging of the support. Preferred example of the method of diselectrification for use in the invention is described in JP-A No. 2002-143747.

Since a non-setting coating solution is used for the image forming layer in the invention, it is important to precisely control the drying air and the drying temperature. Preferred drying method for use in the invention is described in detail in JP-A Nos. 2001-194749 and 2002-139814.

In order to improve film-forming properties in the photothermographic material according to the invention, it is preferred to apply heat treatment immediately after coating and drying. The temperature of the heat treatment is preferably in a range of from 60° C. to 100° C. at the film surface, and the time period for heating is preferably in a range of from 1 sec to 60 sec. More preferably, heating is performed in a temperature range of from 70° C. to 90° C. at the film surface, and the time period for heating is from 2 sec to 10 sec. A preferred method of heat treatment for the invention is described in JP-A No. 2002-107872.

Furthermore, the production methods described in JP-A Nos. 2002-156728 and 2002-182333 are preferably employed in order to produce the photothermographic material according to the invention stably and successively.

The photothermographic material is preferably of mono-sheet type (i.e., a type which forms an image on the photothermographic material without using other sheets such as an image-receiving material).

13) Wrapping Material

In order to suppress fluctuation from occurring on photographic performance during raw stock storage of the photothermographic material according to the invention, or in order to improve curling or winding tendencies when the photothermographic material is manufactured in a roll state, it is preferred that a wrapping material having low oxygen permeability and/or moisture permeability is used. Preferably, oxygen permeability is 50 mL·atm⁻¹m⁻²day⁻¹ or lower at 25° C., more preferably 10 mL·atm⁻¹m⁻²day⁻¹ or lower, and even more preferably 1.0 mL·atm⁻¹m⁻²day⁻¹ or lower. Preferably, moisture permeability is 10 g·atm⁻¹m⁻²day⁻¹ or lower, more preferably 5 g·atm⁻¹m⁻²day⁻¹ or lower, and even more preferably 1 g·atm⁻¹m⁻²day⁻¹ or lower.

As specific examples of a wrapping material having low oxygen permeability and/or moisture permeability, reference can be made to, for instance, the wrapping material described in the specifications of JP-A Nos. 8-254793 and 2000-206653.

14) Other Applicable Techniques

Techniques which can be used for the photothermographic material according to the invention also include those in EP No. 803764A1, EP No. 883022A1, WO No. 98/36322, JP-A Nos. 56-62648 and 58-62644, JP-A Nos. 9-43766, 9-281637, 9-297367, 9-304869, 9-311405, 9-329865, 10-10669, 10-62899, 10-69023, 10-186568, 10-90823, 10-171063, 10-186565, 10-186567, 10-186569 to 10-186572, 10-197974, 10-197982, 10-197983, 10-197985 to 10-197987, 10-207001, 10-207004, 10-221807, 10-282601, 10-288823, 10-288824, 10-307365, 10-312038, 10-339934, 11-7100, 11-15105, 11-24200, 11-24201, 11-30832, 11-84574, 11-65021, 11-109547, 11-125880, 11-129629, 11-133536 to 11-133539, 11-133542, 11-133543, 11-223898, 11-352627, 11-305377, 11-305378, 11-305384, 11-305380, 11-316435, 11-327076, 11-338096, 11-338098, 11-338099, and 11-343420, JP-A Nos. 2001-200414, 2001-234635, 2002-020699, 2001-275471, 2001-275461, 2000-313204, 2001-292844, 2000-324888, 2001-293864, 2001-348546, and 2000-187298.

(Thermal Developing Method)

The temperature for development is preferably from 80° C. to 250° C., more preferably from 100° C. to 140° C., and even more preferably from 110° C. to 130° C. The time period for development is preferably from 1 sec to 60 sec, more preferably from 3 sec to 30 sec, and even more preferably from 5 sec to 25 sec.

In the process of thermal development, either a drum type heater or a plate type heater may be used, although a plate type heater is preferred. A preferable process of thermal development by a plate type heater is a process described in JP-A No. 11-133572, which discloses a thermal developing apparatus in which a visible image is obtained by bringing a photothermographic material with a formed latent image into contact with a heating means at a thermal developing portion, wherein the heating means comprises a plate heater, and a plurality of pressing rollers are oppositely provided along one surface of the plate heater, and the thermal developing apparatus is characterized in that thermal development is performed by passing the photothermographic material between the pressing rollers and the plate heater. It is preferred that the plate heater is divided into 2 to 6 steps, with the leading end having a lower temperature by 1° C. to 10° C. For example, 4 sets of plate heaters which can be independently subjected to the temperature control are used, and are controlled so that they respectively become 112° C., 119° C., 121° C., and 120° C. Such a process is also described in JP-A No. 54-30032, which allows for passage of moisture and organic solvents included in the photothermographic material out of the system, and also allows for suppressing the change in shapes of the support of the photothermographic material upon rapid heating of the photothermographic material.

For downsizing the thermal developing apparatus and for reducing the time period for thermal development, it is preferred that the heater is more stably controlled and that the top part of one sheet of the photothermographic material is exposed and thermal development of the exposed part is started before exposure of the end part of the sheet has completed. Preferable imagers which enable a rapid processing according to the invention are described in, for example, JP-A Nos. 2002-289804 and 2003-285455. Using such imagers, thermal development within 14 sec is possible with a plate type heater having three heating plates which are controlled, for example, at 107° C., 121 ° C. and 121° C., respectively. Thus, the output time period for the first sheet can be reduced to about 60 sec.

(Method for Detecting Chemiluminescence)

In the chemiluminescent enzyme immunoassay method of the present invention, the measurement of a luminous amount of the chemiluminous reaction does not need a device such as a luminous photometer. In the present invention, the luminescence of the chemiluminous reaction is detected by the photothermographic material, and thereafter it is made visible by performing thermal development so that it can be visually sensed. If necessary, it can be shown in numeric values by using an optical densitometer.

As the means of detection using the photothermographic material, for example, (a) a method of coating a solution, that brings about luminous reaction, on the surface of the photothermographic material; (b) a method in which a solution, that brings about luminous reaction, is absorbed to a hydrophilic membrane or the like, followed by adhering it to the photothermographic material; (c) a method in which a solution, that brings about luminous reaction, is applied dropwise to a hydrophilic gel membrane on a board to be absorbed to the gel, followed by adhering the photothermographic material thereon; or the like can be employed.

(a) Method of coating a solution, that brings about luminous reaction, on the surface of the photothermographic material

After preparing the solution that brings about luminous reaction, a certain amount of the solution is picked up using a pipette or the like and applied dropwise on the photothermographic material. After a designated time has past, the photothermographic material is dried and subjected to thermal development.

(b) Method in which a solution that brings about luminous reaction is absorbed to a hydrophilic membrane or the like, followed by adhering it to the photothermographic material

After preparing the solution that brings about luminous reaction, a certain amount of the solution is picked up using a pipette or the like and applied dropwise on a hydrophilic membrane or the like of a certain area to be absorbed into the hydrophilic membrane or the like. Thereafter, the hydrophilic membrane or the like is adhered to the photothermographic material. After a designated time has past, the photothermographic material is dried and subjected to thermal development.

(c) Method in which a solution that brings about luminous reaction is applied dropwise to a hydrophilic gel membrane on a board to be absorbed to the gel, followed by adhering the photothermographic material thereon

A light-reflective board is preferably used as the board. For example, a plastic film in which a light-reflective pigment, for example, a metal oxide such as titanium (IV) oxide, aluminium oxide, or silicon oxide, a salt such as barium sulfate, barium carbonate, calcium carbonate, calcium silicate or the like, or the like is packed, a material in which a film including these light-reflective pigments is coated, or a metal film such as a film of aluminium, silver, iron, aluminium alloy, or the like can be used.

As the gel, a hydrophilic gel is preferred, and for example, a natural product such as gelatin, casein, chitosan, or the like, poly(vinyl alcohol), poly(vinyl alcohol) copolymer, ethyl cellulose, methyl cellulose, CMC sodium salt, acrylic acid polymer, acrylic acid copolymer, or the like can be used.

The gel is disposed on the board by means of coating, printing, or the like. Preferably, the liquid that is applied dropwise is condensed inside a certain area and does not spread to the periphery. Further, it is preferred that the generated light does not diffuse to the periphery and is exposed to the photothermographic material piled thereon.

Therefore, it is preferred that a small hollow portion is prepared on the light-reflective board and the gel is packed inside the hollow portion. The shape, size, and number of the hollow portions are selected freely and vary according the type of the object to be analyzed to be detected, the content thereof, the type of luminescent material, and the like. These are each set preferably from the standpoints of easiness of detection by the photothermographic material, easiness of observation, and the like.

For example, in the immunological inspection using a luminol luminous body, the volume of the gel packed inside the hollow portion is preferably from about 1 μL to 1 mL, and more preferably from about 5 μL to 500 μL. When the volume is fixed, the smaller the area is, the higher the intensity of the emitted light becomes, and the more the inspection accuracy is improved. It depends on the thickness of the board, but for observing the image detected by the photothermographic material visually to carry out judgment, the area is preferably from 0.1 cm² to 10 cm², and more preferably from 0.1 cm² to 1 cm².

Concerning the hollow portion, plural hollow portions may be prepared on the photothermographic material. The shapes of the plural hollow portions prepared may be identical or different from one another. Further, the volumes of each of the plural hollow portions prepared may be identical or different from one another.

Moreover, it is preferred to provide a mechanism to condense the generated light in order to enhance the sensitivity. As the means for condensing the light, for example, a lenticular hollow is prepared on the light-reflective board, and the gel is packed therein. The lenticular shape is preferably such a shape that the focus coincides with the image forming layer of the photothermographic material piled thereon.

The time period for which the solution, in which the luminous reaction occurs, and the photothermographic material are adhered is selected freely, but it is preferable to select a period when the luminous amount is stable and the concentration dependency of the luminous amount is high. For example, the time for starting the measurement is from 0 hours to one hour after mixing the chemicals, preferably from 0 minutes to 30 minutes, and particularly preferably from 0 minutes to 15 minutes, and the time period of adherence is from one second to one minute, preferably from one second to 30 seconds, and particularly preferably from one second to 10 seconds.

After the period has elapsed, the membrane is removed, and the photothermographic material is subjected to drying and thermal development.

Developing processing of the photothermographic material is carried out while adjusting the temperature and time period for heating. The temperature for heating is preferably from 80° C. to 250° C., more preferably from 100° C. to 140° C., and even more preferably from 110° C. to 130° C. The time period for heating is preferably from 1 sec to 60 sec, more preferably from 3 sec to 30 sec, and even more preferably from 5 sec to 25 sec.

### EXAMPLES

The present invention is specifically explained by way of Examples below, which should not be construed as limiting the invention thereto.

### Example 1

### <Preparation of Photothermographic Material>

### 1. Preparation of PET Support

The surface of a biaxially stretched polyethylene terephthalate support having a thickness of 175 μm, on which the photothermographic material was to be coated, was subjected to a corona discharge treatment to prepare a support.

### 2. Image Forming Layer and Surface Protective Layer

### 2-1. Preparation of Coating Materials

1) Silver Halide Emulsion

<<Preparation of Silver Halide Emulsion 1>>

A liquid was prepared by adding 3.1 mL of a 1% by weight solution of potassium bromide, and then 3.5 mL of 0.5 mol/L sulfuric acid and 31.7 g of phthalated gelatin to 1421 mL of distilled water. The liquid was kept at 30° C. while stirring in a stainless-steel reaction vessel, and thereto was added a total amount of: solution A prepared through diluting 22.22 g of silver nitrate by adding distilled water to give the volume of 95.4 mL; and solution B prepared through diluting 15.3 g of potassium bromide and 0.8 g of potassium iodide with distilled water to give the volume of 97.4 mL, over 45 seconds at a constant flow rate. Thereafter, 10 mL of a 3.5% by weight aqueous solution of hydrogen peroxide was added thereto, and 10.8 mL of a 10% by weight aqueous solution of benzimidazole was further added. Moreover, solution C prepared through diluting 51.86 g of silver nitrate by adding distilled water to give the volume of 317.5 mL and solution D prepared through diluting 44.2 g of potassium bromide and 2.2 g of potassium iodide with distilled water to give the volume of 400 mL were added. A controlled double jet method was executed through adding the solution C in its entirety at a constant flow rate over 20 minutes, accompanied by adding the solution D while maintaining the pAg at 8.1. Potassium hexachloroiridate (III) was added in its entirety to give 1×10⁻⁴ mol per 1 mol of silver, at 10 minutes post initiation of the addition of the solution C and the solution D. Moreover, at 5 seconds after completing the addition of the solution C, an aqueous solution of potassium hexacyanoferrate (II) was added in its entirety to give 3×10⁻⁴ mol per 1 mol of silver. The mixture was adjusted to the pH of 3.8 with 0.5 mol/L sulfuric acid. After stopping stirring, the mixture was subjected to precipitation/desalting/water washing steps. The mixture was adjusted to the pH of 5.9 with 1 mol/L sodium hydroxide to produce a silver halide dispersion having the pAg of 8.0.

The above-described silver halide dispersion was kept at 38° C. with stirring, and thereto was added 5 mL of a 0.34% by weight methanol solution of 1,2-benzisothiazolin-3-one, followed by elevating the temperature to 47° C. at 40 minutes thereafter. At 20 minutes after elevating the temperature, sodium benzenethiosulfonate in a methanol solution was added in an amount of 7.6×10⁻⁵ mol per 1 mol of silver. At additional 5 minutes later, tellurium sensitizer C in a methanol solution was added in an amount of 2.9×10⁻⁴mol per 1 mol of silver, and the mixture was subjected to ripening for 91 minutes. Thereafter, a DMF/methanol (1:4) solution of spectral sensitizing dye A was added thereto in an amount of 1.2×10⁻³ mol per 1 mol of silver as the sensitizing dye. At one minute later, 1.3 mL of a 0.8% by weight methanol solution of N,N′-dihydroxy-N″,N″-diethylmelamine was added thereto, and at additional 4 minutes thereafter, 5-methyl-2-mercaptobenzimidazole in a methanol solution in an amount of 4.8×10⁻³ mol per 1 mol of silver, 1-phenyl-2-heptyl-5-mercapto-1,3,4-triazole in a methanol solution in an amount of 5.4×10⁻³ mol per 1 mol of silver, and 1-(3-methylureidophenyl)-5-mercaptotetrazole in an aqueous solution in an amount of 8.5×10⁻³ mol per 1 mol of silver were added to produce silver halide emulsion 1.

Grains in thus prepared silver halide emulsion were silver iodobromide grains having a mean equivalent spherical diameter of 0.042 μm, a variation coefficient of an equivalent spherical diameter distribution of 20%, which uniformly include iodine at 3.5 mol %. Grain size and the like were determined from the average of 1000 grains using an electron microscope. The {100} face ratio of these grains was found to be 80% using a Kubelka-Munk method.

<<Preparation of Silver Halide Emulsion 2>>

Preparation of silver halide emulsion 2 was conducted in a similar manner to the process in the preparation of the silver halide emulsion 1 except that: the temperature of the liquid at the time of grain formation was altered from 30° C. to 60° C.; the solution B was changed to that prepared through diluting 15.9 g of potassium bromide with distilled water to give the v o l u m e of 97.4 mL; the solution D was changed to that prepared through diluting 45.8 g of potassium bromide with distilled water to give the volume of 400 mL; the time period for adding the solution C was changed to 30 minutes; and potassium hexacyanoferrate (II) was deleted. Further, precipitation/desalting/water washing/dispersion were carried out similar to t h e silver halide emulsion 1. Furthermore, spectral sensitization, chemical sensitization, and addition of 5-methyl-2-mercaptobenzimidazole and 1-phenyl-2-heptyl-5-mercapto-1,3,4-triazole were carried out similar to those in the preparation of the silver halide emulsion 1 except that: the amount of the tellurium sensitizer C to be added was changed to 1.1×10⁻⁴ mol per 1 mol of silver; the amount of the DMF/methanol (1:4) solution of the spectral sensitizing dye A to be added was changed to 7.0×10⁻⁴ mol per 1 mol of silver as the sensitizing dye; the addition of 1-phenyl-2-heptyl-5-mercapto-1,3,4-triazole was changed to give 3.3×10⁻³ mol per 1 mol of silver; and the addition of 1-(3-methylureidophenyl)-5-mercaptotetrazole was changed to give 4.7×10⁻³ mol per 1 mol of silver. Thereby, silver halide emulsion 2 was obtained. Grains in the silver halide emulsion 2 were cubic pure silver bromide grains having a mean equivalent spherical diameter of 0.145 μm and a variation coefficient of an equivalent spherical diameter distribution of 20%.

<<Preparation of Silver Halide Emulsion 3>>

Preparation of silver halide emulsion 3 was conducted in a similar manner to the process in the preparation of the silver halide emulsion 1 except that the temperature of the liquid at the time of grain formation was altered from 30° C. to 27° C. Further, precipitation/desalting/water washing/dispersion were carried out similar to the silver halide emulsion 1. Furthermore, spectral sensitization and chemical sensitization were carried out similar to those in the preparation of the silver halide emulsion 1 except that: the addition amount of the DMF/methanol (1:4) solution of the spectral sensitizing dye A was changed to 1.5×10⁻³ mol per 1 mol of silver as the sensitizing dye, and the addition amount of the tellurium sensitizer C was changed to 3.6×10⁻⁴ mol per 1 mol of silver. Thereby, silver halide emulsion 3 was obtained. Grains in the silver halide emulsion 3 were silver iodobromide grains having a mean equivalent spherical diameter of 0.034 μm and a variation coefficient of an equivalent spherical diameter distribution of 20%, which uniformly include iodine at 3.5 mol %.

<<Preparation of Mixed Emulsion for Coating Solution>>

The silver halide emulsion 1, the silver halide emulsion 2, and the silver halide emulsion 3 was mixed at a ratio of 10% by weight, 85% by weight, and 5% by weight, respectively. Further, water was added thereto to give the content of silver halide of 38.2 g per 1 kg of the mixed emulsion for a coating solution.

2) Preparation of Dispersion of Silver Salt of Fatty Acid

<Preparation of Recrystallized Behenic Acid>

Behenic acid manufactured by Henkel Co. (trade name: Edenor C22-85R) in an amount of 100 kg was admixed with 1200 kg of isopropyl alcohol, and dissolved at 50° C. The mixture was filtrated through a 10 μm filter, and cooled to 30° C. to allow recrystallization. Cooling speed for the recrystallization was controlled to be 3° C./hour. The resulting crystal was subjected to centrifugal filtration, and washing was performed with 100 kg of isopropyl alcohol. Thereafter, the crystal was dried. The resulting crystal was esterified, and subjected to GC-FID analysis to give the result of the content of behenic acid being 96%. In addition, lignoceric acid, arachidic acid, and erucic acid were included in an amount of 2%, 2%, and 0.001%, respectively.

<Preparation of Dispersion of Silver Salt of Fatty Acid>

88 kg of the recrystallized behenic acid, 422 L of distilled water, 49.2 L of 5 mol/L sodium hydroxide aqueous solution, and 120 L of t-butyl alcohol were admixed, and subjected to reaction with stirring at 75° C. for one hour to provide a solution of sodium behenate. Separately, 206.2 L of an aqueous solution containing 40.4 kg of silver nitrate (pH 4.0) was provided, and kept at a temperature of 10° C. A reaction vessel charged with 635 L of distilled water and 30 L of t-butyl alcohol was kept at 30° C., and thereto were added the entire amount of the solution of sodium behenate and the entire amount of the aqueous solution of silver nitrate with sufficient stirring at a constant flow rate over 93 minutes and 15 seconds, and 90 minutes, respectively.

In this process, during first 11 minutes following the initiation of adding the aqueous solution of silver nitrate, the added material was restricted to the aqueous solution of silver nitrate alone. The addition of the solution of sodium behenate was thereafter started, and during 14 minutes and 15 seconds following the completion of adding the aqueous solution of silver nitrate, the added material was restricted to the solution of sodium behenate alone. In this process, the temperature inside of the reaction vessel was set to be 30° C. and the temperature outside was controlled so that the temperature of the liquid was kept constant. In addition, the temperature of a pipeline for the addition system of the solution of sodium behenate was kept constant by circulation of warm water outside of a double wall pipe, so that the temperature of the liquid at an outlet in the leading edge of the nozzle for addition was adjusted to be 75° C. Further, the temperature of a pipeline for the addition system of the aqueous solution of silver nitrate was kept constant by circulation of cool water outside of a double wall pipe. The position at which the solution of sodium behenate was added and the position at which the aqueous solution of silver nitrate was added were arranged symmetrically with a shaft for stirring located at a center. Moreover, both of the positions were adjusted to avoid contact with the reaction liquid.

After completing the addition of the solution of sodium behenate, the mixture was left to stand at the temperature as it was for 20 minutes while stirring. The temperature of the mixture was then elevated to 35° C. over 30 minutes followed by ripening for 2.10 minutes. Immediately after completing the ripening, solid matters were filtered out with centrifugal filtration. The solid matters were washed with water until the electric conductivity of the filtrated water became 30 μS/cm. Thereby, a silver salt of a fatty acid was obtained. The resulting solid matters were stored as a wet cake without drying.

When the shape of the resulting particles of silver behenate was evaluated by electron micrography, a crystal was revealed having a=0.21 μm, b=0.4 μm and c=0.4 μm on the average value, with a mean aspect ratio of 2.1, and a variation coefficient of an equivalent spherical diameter distribution of 11% (a, b, and c are as defined aforementioned.).

To the wet cake corresponding to 260 kg of a dry solid matter content, were added 19.3 kg of poly(vinyl alcohol) (trade name: PVA-217) and water to give the total amount of 1000 kg. Then, slurry was obtained from the mixture using a dissolver blade. Additionally, the slurry was subjected to preliminary dispersion with a pipeline mixer (manufactured by MIZUHO Industrial Co., Ltd.: PM-10 type).

Next, a stock liquid after the preliminary dispersion was treated three times using a dispersing machine (trade name: Microfluidizer M-610, manufactured by Microfluidex International Corporation, using Z type Interaction Chamber) with the pressure controlled to be 1150 kg/cm² to provide a dispersion of silver behenate. For the cooling operation, coiled heat exchangers were equipped in front of and behind the interaction chamber respectively, and accordingly, the temperature for the dispersion was set to be 18° C. by regulating the temperature of the cooling medium.

3) Preparation of Reducing Agent Dispersion

To 10 kg of reducing agent-I (6,6′-di-t-butyl-4,4′-dimethyl-2,2′-butylidenediphenol)) and 16 kg of a 10% by weight aqueous solution of modified poly(vinyl alcohol) (manufactured by Kuraray Co., Ltd., Poval MP-203) was added 10 kg of water, and thoroughly mixed to give slurry. This slurry was fed with a diaphragm pump, and was subjected to dispersion with a horizontal sand mill (UVM-2: manufactured by AIMEX Co., Ltd.) packed with zirconia beads having a mean particle diameter of 0.5 mm for 3 hours. Thereafter, 0.2 g of benzisothiazolinone sodium salt and water were added thereto, thereby adjusting the concentration of the reducing agent to be 25% by weight. This dispersion was subjected to heat treatment at 60° C. for 5 hours to obtain reducing agent-1 dispersion.

Particles of the reducing agent included in the resulting reducing agent dispersion had a median diameter of 0.40 μm, and a maximum particle diameter of 1.4 μm or less. The resulting reducing agent dispersion was subjected to filtration with a polypropylene filter having a pore size of 3.0 μm to remove foreign substances such as dust, and stored.

4) Preparation of Hydrogen Bonding Compound Dispersion

To 10 kg of hydrogen bonding compound-I (tri(4-t-butylphenyl)phosphineoxide) and 16 kg of a 10% by weight aqueous solution of modified poly(vinyl alcohol) (manufactured by Kuraray Co., Ltd., Poval MP-203) was added 10 kg of water, and thoroughly mixed to give slurry. This slurry was fed with a diaphragm pump, and was subjected to dispersion with a horizontal sand mill (UVM-2: manufactured by AIMEX Co., Ltd.) packed with zirconia beads having a mean particle diameter of 0.5 mm for 4 hours. Thereafter, 0.2 g of benzisothiazolinone sodium salt and water were added thereto, thereby adjusting the concentration of the hydrogen bonding compound to be 25% by weight. This dispersion was heated at 40° C. for one hour, followed by a subsequent heat treatment at 80° C. for one hour to obtain hydrogen bonding compound-1 dispersion. Particles of the hydrogen bonding compound included in the resulting hydrogen bonding compound dispersion had a median diameter of 0.45 μm, and a maximum particle diameter of 1.3 μm or less. The resulting hydrogen bonding compound dispersion was subjected to filtration with a polypropylene filter having a pore size of 3.0 μm to remove foreign substances such as dust, and stored.

5) Preparation of Development Accelerator Dispersion

<Preparation of Development Accelerator-1 Dispersion>

To 10 kg of development accelerator-I and 20 kg of a 10% by weight aqueous solution of modified poly(vinyl alcohol) (manufactured by Kuraray Co., Ltd., Poval MP-203) was added 10 kg of water, and thoroughly mixed to give slurry. This slurry was fed with a diaphragm pump, and was subjected to dispersion with a horizontal sand mill (UVM-2: manufactured by AIMEX Co., Ltd.) packed with zirconia beads having a mean particle diameter of 0.5 mm for 3 hours and 30 minutes. Thereafter, 0.2 g of benzisothiazolinone sodium salt and water were added thereto, thereby adjusting the concentration of the development accelerator to be 20% by weight. Accordingly, development accelerator-1 dispersion was obtained. Particles of the development accelerator included in the resulting development accelerator dispersion had a median diameter of 0.48 μm, and a maximum particle diameter of 1.4 μm or less. The resulting development accelerator dispersion was subjected to filtration with a polypropylene filter having a pore size of 3.0 μm to remove foreign substances such as dust, and stored.

Also concerning solid dispersion of development accelerator-2, dispersion was executed similar to that in the development accelerator-1, and thereby a dispersion of 20% by weight was obtained.

6) Preparation of Organic Polyhalogen Compound Dispersion

<Preparation of Organic Polyhalogen Compound-1 Dispersion>

10 kg of organic polyhalogen compound-1 (tribromomethane sulfonylbenzene), 10 kg of a 20% by weight aqueous solution of modified poly(vinyl alcohol) (manufactured by Kuraray Co., Ltd., Poval MP-203), 0.4 kg of a 20% by weight aqueous solution of sodium triisopropylnaphthalenesulfonate and 14 kg of water were thoroughly admixed to give slurry. This slurry was fed with a diaphragm pump, and was subjected to dispersion with a horizontal sand mill (UVM-2: manufactured by AIMEX Co., Ltd.) packed with zirconia beads having a mean particle diameter of 0.5 mm for 5 hours. Thereafter, 0.2 g of benzisothiazolinone sodium salt and water were added thereto, thereby adjusting the concentration of the organic polyhalogen compound to be 30% by weight. Accordingly, organic polyhalogen compound-1 dispersion was obtained. Particles of the organic polyhalogen compound included in the resulting organic polyhalogen compound dispersion had a median diameter of 0.41 μm, and a maximum particle diameter of 2.0 μm or less. The resulting organic polyhalogen compound dispersion was subjected to filtration with a polypropylene filter having a pore size of 10.0 μm to remove foreign substances such as dust, and stored.

<Preparation of Organic Polyhalogen Compound-2 Dispersion>

10 kg of organic polyhalogen compound-2 (N-butyl-3-tribromomethane sulfonylbenzamide), 20 kg of a 10% by weight aqueous solution of modified poly(vinyl alcohol) (manufactured by Kuraray Co., Ltd., Poval MP-203) and 0.4 kg of a 20% by weight aqueous solution of sodium triisopropylnaphthalenesulfonate were thoroughly admixed to give slurry. This slurry was fed with a diaphragm pump, and was subjected to dispersion with a horizontal sand mill (UVM-2: manufactured by AIMEX Co., Ltd.) packed with zirconia beads having a mean particle diameter of 0.5 mm for 5 hours. Thereafter, 0.2 g of benzisothiazolinone sodium salt and water were added thereto, thereby adjusting the concentration of the organic polyhalogen compound to be 30% by weight. This dispersion was warmed at 40° C. for 5 hours to obtain organic polyhalogen compound-2 dispersion. Particles of the organic polyhalogen compound included in the resulting organic polyhalogen compound dispersion had a median diameter of 0.40 μm, and a maximum particle diameter of 1.3 μm or less. The resulting organic polyhalogen compound dispersion was subjected to filtration with a polypropylene filter having a pore size of 3.0 μm to remove foreign substances such as dust, and stored.

7) Preparation of Phthalazine Compound Solution

Modified poly(vinyl alcohol) MP-203 manufactured by Kuraray Co., Ltd. in an amount of 8 kg was dissolved in 174.57 kg of water, and then, thereto were added 3.15 kg of a 20% by weight aqueous solution of sodium triisopropylnaphthalenesulfonate and 14.28 kg of a 70% by weight aqueous solution of 6-isopropyl phthalazine to prepare a 5% by weight solution.

8) Preparation of Solid Fine Particle Dispersion of Nucleator X-1

To 4 kg of nucleator X-1 were added 1 kg of modified poly(vinyl alcohol) (manufactured by Kuraray Co., Ltd., Poval PVA-217) and 36 kg of water, and thoroughly mixed to give slurry. This slurry was fed with a diaphragm pump, and was subjected to dispersion with a horizontal sand mill (UVM-2: manufactured by AIMEX Co., Ltd.) packed with zirconia beads having a mean particle diameter of 0.5 mm for 13 hours. Thereafter, 4 g of benzisothiazolinone sodium salt and water were added thereto, thereby adjusting the concentration of the nucleator to be 10% by weight. Thereby, a solid fine particle dispersion of the nucleator X-1 was obtained. Particles of the nucleator included in the resulting dispersion had a mean particle size of 0.33 μm, a maximum particle diameter of 3.0 μm or less, and a variation coefficient of a particle size distribution of 24%. The resulting dispersion was subjected to filtration with a polypropylene filter having a pore size of 3.0 μm to remove foreign substances such as dust, and stored.

9) Preparation of Dispersion A of Silver Salt of Benzotriazole

1 kg of benzotriazole was added to a liquid prepared by dissolving 360 g of sodium hydroxide in 9100 mL of water, and then the mixture was stirred for 60 minutes. Thereby, solution BT of sodium salt of benzotriazole was prepared. A liquid prepared by dissolving 55.9 g of alkali-processed de-ionized gelatin in 1400 mL of distilled water was kept at 70° C. while stirring in a stainless-steel reaction vessel. And then, solution A prepared through diluting 54.0 g of silver nitrate by adding distilled water to give the volume of 400 mL, and solution B prepared through diluting 397 mL of the solution BT of sodium salt of benzotriazole with distilled water to give the volume of 420 mL were added. A method of double jet was executed through adding 220 mL of the solution B at a constant flow rate of 20 mL/min over 11 minutes to the stainless-steel reaction vessel, and at one minute post initiation of the addition of the solution B, 200 mL of the solution A was added thereto at a constant flow rate of 20 mL/min over 10 minutes. Moreover, at 6 minutes later after completing the addition, the solution A and the solution B were added simultaneously at a constant flow rate of 33.34 mL/min over 6 minutes in an amount of 200 mL respectively. The mixture was cooled to 45° C., and 92 mL of Demol N (10% aqueous solution, manufactured by Kao Corporation) was added to the mixture while stirring. The mixture was adjusted to the pH of 4.1 with 1 mol/L sulfuric acid. After stopping stirring, the mixture was subjected to precipitation/desalting/water washing steps.

Thereafter, the resulting mixture was warmed to 50° C. and 51 mL of 1 mol/L sodium hydroxide was added thereto while stirring, and then 11 mL of a methanol solution of benzoisothiazolinone (3.5%) and 7.7 mL of a methanol solution of sodium benzenethiosulfonate (1%) were added thereto. After stirring the mixture for a period of 80 minutes, the mixture was adjusted to the pH of 7.8 with 1 mol/L sulfuric acid. Thereby, dispersion A of silver salt of benzotriazole was prepared.

Particles of the prepared dispersion of silver salt of benzotriazole had a mean equivalent circular diameter of 0.172 μm, a variation coefficient of an equivalent circular diameter distribution of 18.5%, a mean length of long side of 0.32 μm, a mean length of short side of 0.09 μm, and a mean ratio of the length of short side to the length of long side of 0.298. Particle size and the like were determined from the average of 300 particles using an electron microscope.

10) Preparation of Solution of Additive

<Preparation of Aqueous Solution of Mercapto Compound-1>

Mercapto compound-1 (1-(3-sulfophenyl)-5-mercaptotetrazole sodium salt) in an amount of 7 g was dissolved in 993 g of water to provide a 0.7% by weight aqueous solution.

<Preparation of Aqueous Solution of Mercapto Compound-2>

Mercapto compound-2 (1-(3-methylureidophenyl)-5-mercaptotetrazole) in an amount of 20 g was dissolved in 980 g of water to provide a 2.0% by weight aqueous solution.

<Preparation of Aqueous Solution of Phthalic Acid>

A 20% by weight aqueous solution of diammonium phthalate was prepared.

11) Preparation of SBR Latex Liquid

Into a polymerization vessel of a gas monomer reaction apparatus (manufactured by Taiatsu Techno Corporation, TAS-2J type) were poured 287 g of distilled water, 7.73 g of surfactant (PIONIN A-43-S (manufactured by TAKEMOTO OIL & FAT CO., LTD.): solid matter content of 48.5% by weight), 14.06 mL of 1 mol/L sodium hydroxide, 0.15 g of ethylenediamine tetraacetate tetrasodium salt, 255 g of styrene, 11.25 g of acrylic acid, and 3.0 g of tert-dodecyl mercaptan, followed by sealing of the reaction vessel and stirring at a stirring rate of 200 rpm. Degassing was conducted with a vacuum pump, followed by repeating nitrogen gas replacement several times. Thereto was injected 108.75 g of 1,3-butadiene, and the inner temperature of the vessel was elevated to 60° C. Thereto was added a solution obtained by dissolving 1.875 g of ammonium persulfate in 50 mL of water, and the mixture was stirred for 5 hours as it stands. Further, the mixture was heated to 90° C., followed by stirring for 3 hours. After completing the reaction, the inner temperature of the vessel was lowered to reach to the room temperature, and thereafter the mixture was treated by adding 1 mol/L sodium hydroxide and ammonium hydroxide to give the molar ratio of Na⁺ ion:NH₄⁺ ion=1: 5.3, and thus, the pH of the mixture was adjusted to 8.4. Thereafter, filtration with a polypropylene filter having a pore size of 1.0 μm was conducted to remove foreign substances such as dust, and stored. Thereby, SBR latex was obtained in an amount of 774.7 g.

The aforementioned latex had a mean particle diameter of 90 nm, Tg of 17° C., a solid content of 44% by weight, an equilibrium moisture content at 25° C. and 60% RH of 0.6% by weight, an ionic conductivity of 4.80 mS/cm (measurement of the ionic conductivity was performed using a conductometer CM-30S manufactured by Toa Electronics Ltd. for the latex stock solution (44% by weight) at 25° C.), and the pH of 8.4.

2-2. Preparation of Coating Solutions

1) Preparation of Coating Solution for Image Forming Layer

To the dispersion of silver salt of a fatty acid in an amount of 1000 g were serially added water, the organic polyhalogen compound-1 dispersion, the organic polyhalogen compound-2 dispersion, the SBR latex liquid, the reducing agent-1 dispersion, the hydrogen bonding compound-1 dispersion, the development accelerator-1 dispersion, the development accelerator-2 dispersion, the phthalazine compound solution, the mercapto compound-1 aqueous solution, and the mercapto compound-2 aqueous solution. By adding, just prior to coating, the mixed emulsion for a coating solution thereto and mixing sufficiently, a coating solution for the image forming layer was prepared and allowed to be transported to a coating die and coated.

2) Preparation of Coating Solution for First Layer of Surface Protective Layers

In 704 mL of water were dissolved 100 g of inert gelatin and 10 mg of benzisothiazolinone, and thereto were added 146 g of the dispersion A of silver salt of benzotriazole, 180 g of a 19% by weight liquid of methyl methacrylate/styrene/butyl acrylate/hydroxyethyl methacrylate/acrylic acid copolymer (weight ratio of the copolymerization of 57/8/28/5/2) latex, 46 mL of a 15% by weight methanol solution of phthalic acid, and 5.4 mL of a 5% by weight aqueous solution of di(2-ethylhexyl) sodium sulfosuccinate, and were mixed. By adding, just prior to coating, 40 mL of a 4% by weight chrome alum thereto and mixing with a static mixer, a coating solution for the first layer of the surface protective layers was prepared, which was fed to a coating die so that the amount of the coating solution became 35 mL/m².

Viscosity of the coating solution was 20 [mPa·s] which was measured with a B type viscometer at 40° C. (No. 1 rotor, 60 rpm).

3) Preparation of Coating Solution for Second Layer of Surface Protective Layers

In water was dissolved 80 g of inert gelatin and thereto were added 102 g of a 27.5% by weight liquid of methyl methacrylate/styrene/butyl acrylate/hydroxyethyl methacrylate/acrylic acid copolymer (weight ratio of the copolymerization of 64/9/20/5/2) latex, 5.4 mL of a 2% by weight solution of fluorocarbon surfactant (F-1), 5.4 mL of a 2% by weight aqueous solution of fluorocarbon surfactant (F-2), 23 mL of a 5% by weight aqueous solution of aerosol OT (manufactured by American Cyanamid Co.), 4 g of poly(methyl methacrylate) fine particles (mean particle diameter of 0.7 μm, distribution of volume-weighted average of 30%), 21 g of poly(methyl methacrylate) fine particles (mean particle diameter of 3.6 μm, distribution of volume-weighted average of 60%), 1.6 g of 4-methyl phthalic acid, 4.8 g of phthalic acid, 44 mL of 0.5 mol/L sulfuric acid, and 10 mg of benzisothiazolinone. Water was added to give the total amount of 650 g. Just prior to coating, 445 mL of an aqueous solution containing 4% by weight chrome alum and 0.67% by weight phthalic acid was added and admixed with a static mixer to provide a coating solution for the second layer of the surface protective layers, which was fed to a coating die to provide 8.3 mL/m².

Viscosity of the coating solution was 19 [mPa·s] which was measured with a B type viscometer at 40° C. (No. 1 rotor, 60 rpm).

### 3. Preparation of Photothermographic Materials

1) Preparation of Photothermographic Material A

Simultaneous multilayer coating was carried out by a slide bead coating method in order of the image forming layer, first layer of the surface protective layers, and second layer of the surface protective layers, starting from the support, and thereby photothermographic material A was produced.

The coating amount of each compound (g/m²) for the image forming layer is as follows.

| | |
|---|---|
| Silver salt of a fatty acid (on the basis of silver content) | 0.745 |
| Organic polyhalogen compound-1 | 0.14 |
| Organic polyhalogen compound-2 | 0.28 |
| Phthalazine compound | 0.18 |
| SBR latex | 9.43 |
| Reducing agent-1 | 0.77 |
| Hydrogen bonding compound-1 | 0.112 |
| Development accelerator-1 | 0.019 |
| Development accelerator-2 | 0.016 |
| Mercapto compound-2 | 0.003 |
| Silver halide (on the basis of Ag content) | 0.12 |

2) Preparation of Photothermographic Material B

Simultaneous multilayer coating was carried out by a slide bead coating method in order of the image forming layer, first layer of the surface protective layers, and second layer of the surface protective layers, starting from the support, and thereby photothermographic material B was produced.

The coating amount of each compound (g/m²) for the image forming layer is as follows.

| | |
|---|---|
| Silver salt of a fatty acid (on the basis of silver content) | 0.805 |
| Organic polyhalogen compound-1 | 0.088 |
| Organic polyhalogen compound-2 | 0.088 |
| Phthalazine compound | 0.18 |
| SBR latex | 9.43 |
| Reducing agent-1 | 0.77 |
| Hydrogen bonding compound-1 | 0.112 |
| Development accelerator-1 | 0.019 |
| Development accelerator-2 | 0.012 |
| Mercapto compound-2 | 0.003 |
| Silver halide (on the basis of Ag content) | 0.060 |
| Nucleator X-1 | 0.100 |

Chemical structures of the compounds used in Examples of the invention are shown below.

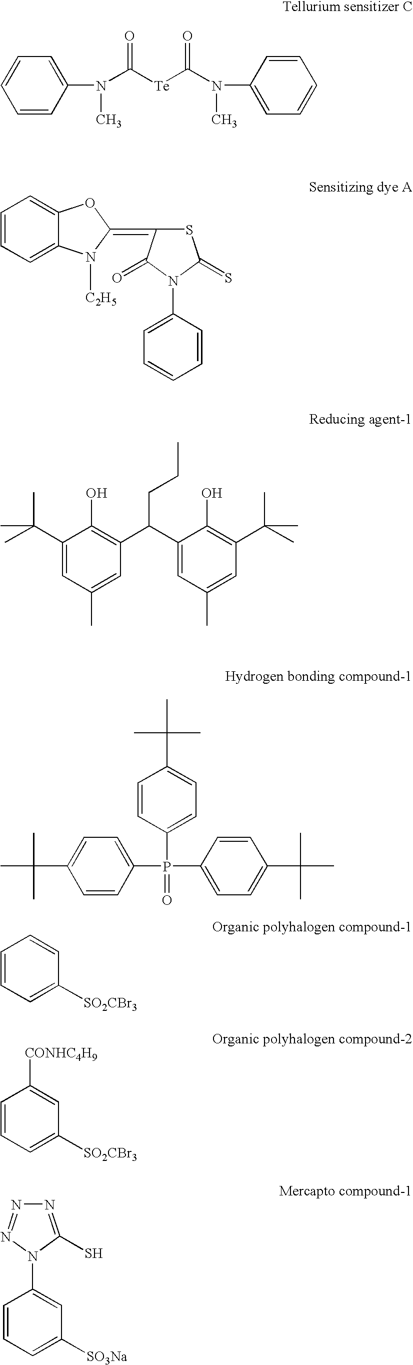

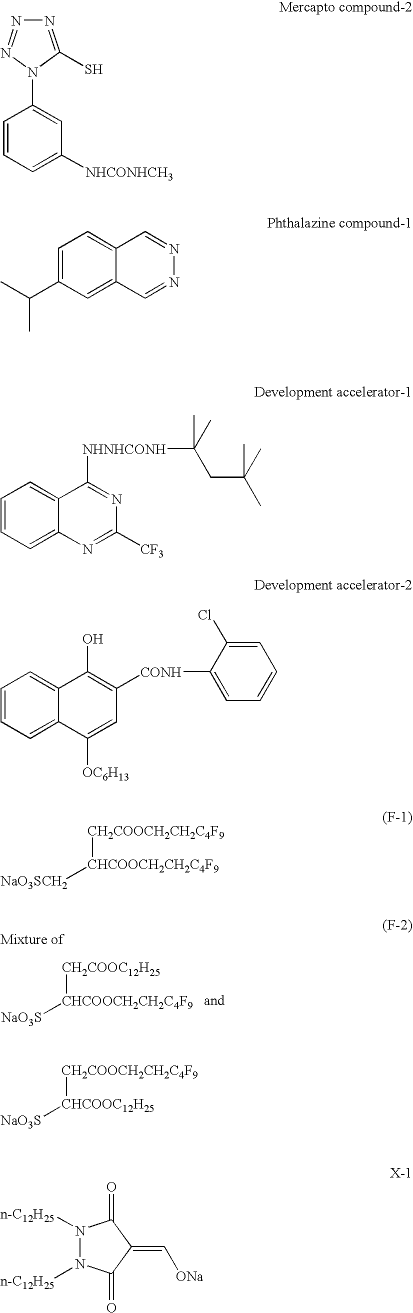

<Preparation of Biotin Antibody>

Biotin labeling of anti-CRP antibody (Clone No. M701289, available from Fitzgerald Industries International Inc.) was carried out using commercially available kit (Biotin Labeling kit-SH, manufactured by DOJINDO).

<Preparation of Magnetic Beads-labeled Antibody>

Preparation of magnetic beads-labeled anti-CRP antibody was conducted by reacting magnetic beads coated by streptavidin (available from Ademtech SA Corp., particle size of 0.2 μm) with the biotin anti-CRP antibody prepared above in a combined buffer (including Tris-Cl in an amount of 5 mM, EDTA in an amount of 0.1 mM, and sodium chloride in an amount of 0.5 mM) for two hours. Recovery by a magnet and washing were repeated, and after removing the unbonded antibody, blocking was performed using BSA in order to prevent non-specific adsorption.

<Preparation of Peroxidase-labeled Antibody>

Peroxidase labeling of anti-CRP antibody (Clone No. M7111422, available from Fitzgerald Industries International Inc.) was carried out by using commercially available kit (Peroxidase Labeling kit-SH, manufactured by DOJINDO).

<Detection of CRP>

To a well-shaped reaction vessel (φ6.5 mm×11.2 mm (H)) were added 10 μL of a magnetic beads-labeled antibody solution in a concentration of 0.1 μg/mL (1 nM) on the basis of protein, 10 μL of a 0.5 μg/mL (1 nM) solution of the peroxidase-labeled antibody, and 10 μL of the sample including CRP at a concentration shown in Table 1, followed by incubating the vessel at 37° C. for 30 minutes. The formed complex was recovered by a magnet and washing was repeated using physiological saline including 0.05% by weight Tween 20 (ICI Chemicals & Polymers, a salt of polyoxyethylene monolauric acid (average repeating number of repeating units of 20)) to remove the unbonded peroxidase-labeled antibody, preparing to be 10 μL in the end. Thereafter, a luminol-type luminous substrate solution and hydrogen peroxide solution (all manufactured by PIERCE Corp., Super Signal (registered trademark) West Femto Maximum Sensitivity Substrate kit, emission wavelength of 425 nm) were each added in an amount of 10 μL to start luminous reaction.

Just after starting the reaction, the entire reaction solution was applied dropwise on the photothermographic materials A and B using micropipettes, and the portion on which the reaction solution was applied dropwise was covered with an aluminium cover. After the reaction was completed, the photothermographic material was adhered to a plate heater heated at 120° C. for 14 seconds. The change in density was observed visually, compared with the sample in which CRP was not added as a control sample, and evaluated whether difference is seen or not. As a comparison of the measuring method, the luminescence of well was measured using a microplate reader (ARVOMX, manufactured by Perkin Elmer Corp.), and the region possible to measure the CRP concentration was compared with the present invention. The obtained results are shown in Table 1.

TABLE 1

| | | | Judgment |
|---|---|---|---|
| | | | with respect to |
| | | | Existence or |
| CRP | Detection of Concentration by | | Nonexistence |
| Concentration | Photothermographic Material | | of CRP by |
| (pM) | Sample A | Sample B | Microplate Reader |
| 100 | possible | possible | possible |
| 10 | possible | possible | possible |
| 1 | impossible | possible | impossible |

As is shown in Table 1, the present invention makes it possible to detect the existence of antigen only by heating and without using a special spectroscope. It is also realized that the sample B using the nucleator can detect the object highly precisely.

### Example 2

<Preparation of Antigen Solid-phased Plate>

Anti-CRP antibody (Clone No. M701289, available from Fitzgerald Industries International Inc.) was prepared to be 10 μg/mL using 150 mM sodium chloride. 100 μL of this solution was respectively added to 96 well microplate (available from NUNC, Maxisorp), followed by incubating at room temperature for two hours. After removing the antibody solution, as a blocking agent for preventing non-specific adsorption, 300 μL of PBS (pH 7.4) containing 1% by weight of casein was respectively added, followed by incubating at room temperature for one hour. Thereafter, the blocking agent was removed to prepare an anti-CRP antibody solid-phased microplate.

<(1) Preparation of CHM Amylase>

5 mg of α-amylase derived from Bacillus subtilis (available from Fluka Corp.) was dissolved in 1 mL of 0.1 M glycerophosphoric acid having the pH of 6.3. Then, 100 mL of a 2 mg/mL DMF solution of [4-(maleimidomethyl)cyclohexane-1-carboxylic acid] succinimide ester (CHMS) (MP, Biomedicals, Inc.) was added thereto and was allowed to react at room temperature for one hour. This reaction liquid was applied to a Sephadex G-25 column (MP, Biomedicals, Inc.) and 0.1 M glycerophosphoric acid having the pH of 6.3 was passed through the column to portion out the through-out fraction. Thereby, 4-(maleimidomethyl)cyclohexane-1-carboxyamido α-amylase (CHM α-amylase) was obtained.

<(2) Preparation of Anti-CRP Mouse IgG F(ab′)₂>

300 μg of papain was added to 2 mL of a 0.1 M acetic acid buffer solution (pH 5.5) containing 10 mg of anti-CRP mouse IgG and stirred at 37° C. for 18 hours. The reaction liquid was adjusted to the pH of 6.0 using 0.1 N sodium hydroxide and then was applied to an AcA-44 gel column (Sigma) previously buffered with a 0.1M phosphoric acid buffer solution (containing 1 mM EDTA, pH 6.0), followed by elution with the aforementioned phosphoric acid buffer solution. The peak portion of the eluate including substances having molecular weights of about 100,000 was collected and concentrated to be 1 mL. Thereby, the objective anti-CRP mouse IgG F(ab′)₂ was obtained.

<(3) Preparation of α-Amylase-labeled Antibody>

To 1 mL of a 0.1 M phosphoric acid buffer solution (containing 1 mM EDTA, pH 6.0) containing 6 mg of anti-CRP mouse IgG F(ab′)₂ prepared in (2) was added 100 μL of a 10 mg/mL aqueous solution of 2-mercaptoethylamine hydrochloric acid salt and stirred at 37° C. for 90 minutes. Then, the reaction liquid was subjected to gel filtration using a Sephadex G-25 column previously buffered with a 0.1M phosphoric acid buffer solution (pH 6.3) to remove unreacted 2-mercaptoethylamine. Thereby, HS-Fab′ was obtained. To the obtained HS-Fab′ was added 2 mg of CHM α-amylase prepared in (1) and the mixture was allowed to react at 37° C. for 90 minutes. Thereafter, this reaction liquid was subjected to gel filtration using an AcA-34 column (Sigma) buffered with a 0.1 M acetic acid buffer solution (containing 5 mM calcium chloride, pH 7.0). The fraction including substances having molecular weights of 200,000 or more was collected and concentrated. Thereby, the objective α-amylase-labeled antibody was obtained.

<Synthesis of Amylase-active Substrate for Measurement (Biotin-G7PAP-nucleator)>

Synthesis of α-amylase-active substrate for measurement was carried out according to the following synthetic path.

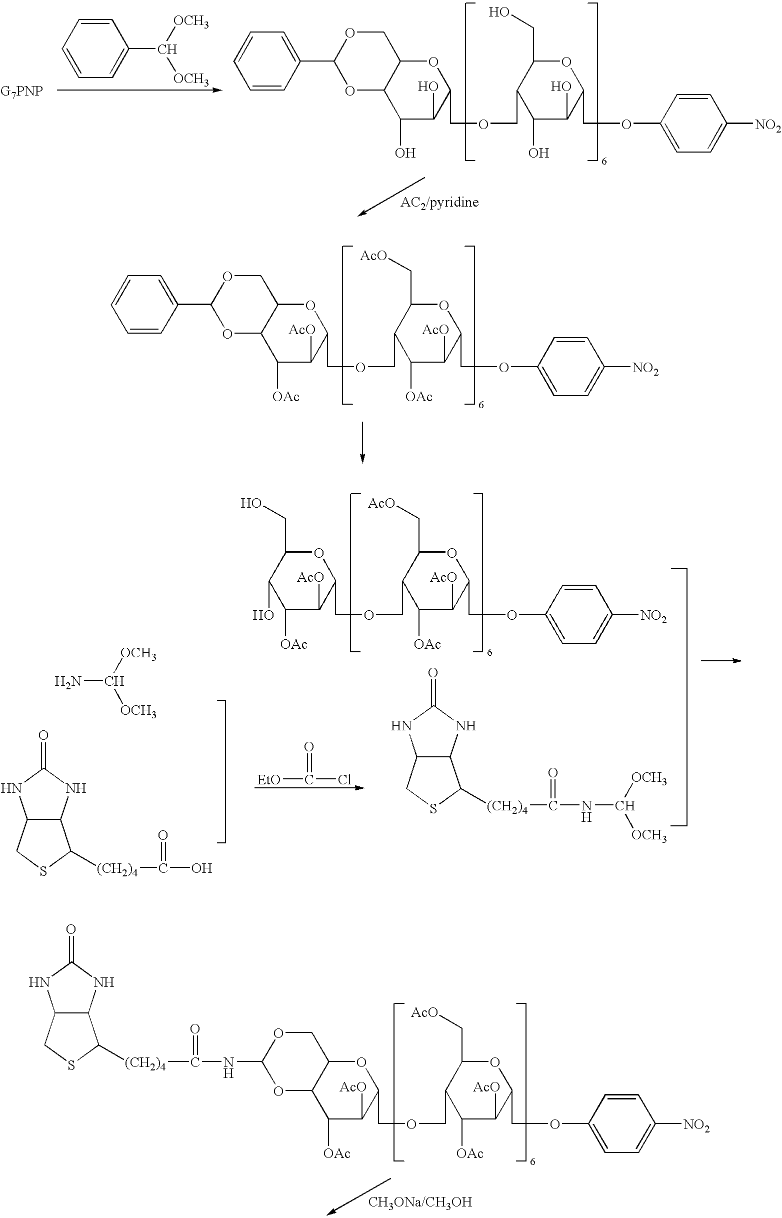

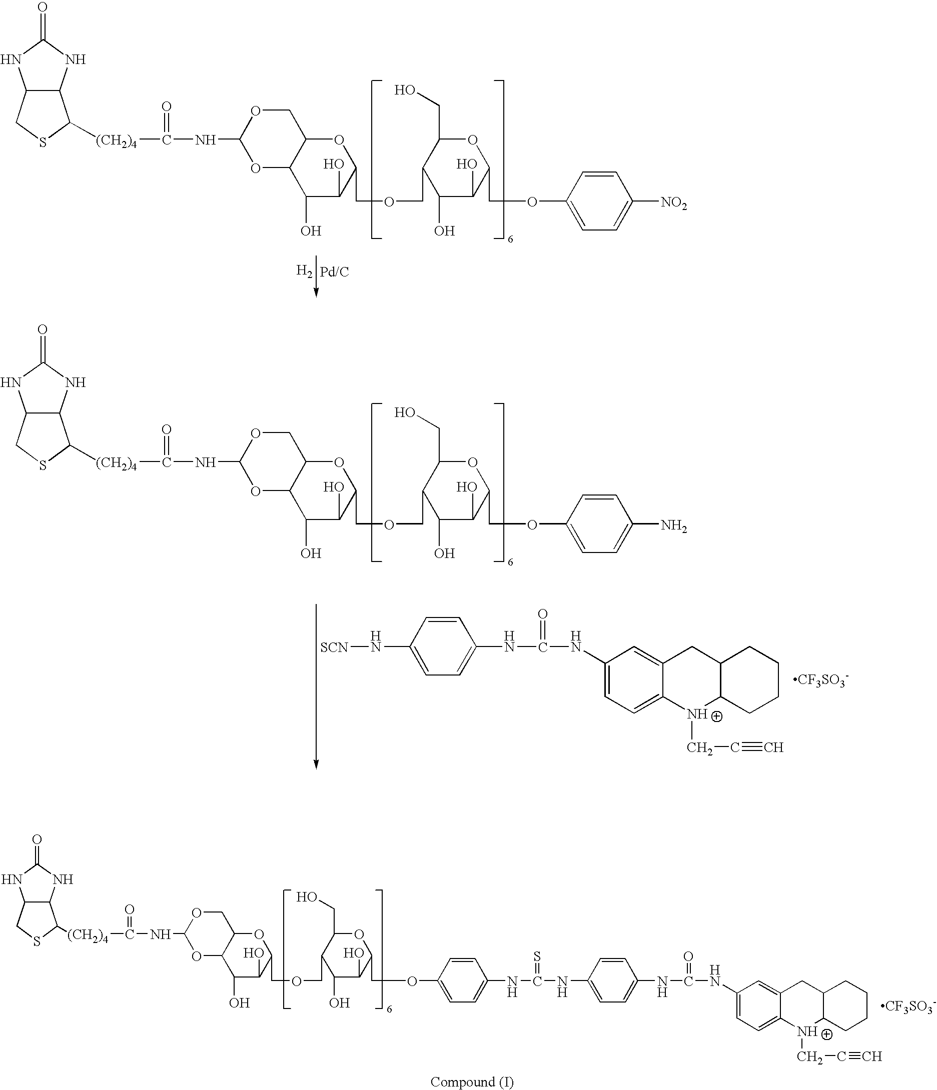

<Detection of CRP>

To the anti-CRP antibody solid-phased microplate were added 50 μL of a 0.5 μg/mL (2.5 nM) solution of the α-amylase-labeled antibody and 50 μL of the sample including CRP at a concentration shown in Table 2, followed by incubating at 37° C. for 30 minutes. After removing the reaction solution, washing was repeated using physiological saline including 0.05% by weight Tween 20 (registered trademark, ICI Chemicals & Polymers) to remove the unbonded α-amylase-labeled antibody. Thereafter, 50 μL of a 1.0×10⁻⁴ M solution of α-amylase substrate (solution of the compound (I)) (prepared using a phosphoric acid buffer solution (pH 6.0)), followed by incubating at 37° C. for 30 minutes.

50 μL of the substrate solution after reaction was charged to a small column of avidin agarose gel (gel volume of 100 μL, biotin bonding ability of 0.5 μg) which was previously buffered with 125 mM pyridine acetic acid solution (pH 6.0) containing 0.5 M of sodium chloride, and further, 50 μL of the above-described pyridine acetic acid solution (pH 6.0) was applied on the upper end of the column, followed by eluting liquid by air pressure to recover a solution in which surplus substrates were removed. Then, 30 μL of the recovered solution was applied dropwise on the photothermographic materials A and B prepared in Example 1 using micropipettes. After the reaction was completed, the photothermographic material was adhered to a plate heater heated at 120° C. for 14 seconds. The change in density was observed visually, compared with the sample in which CRP was not added as a control sample, and evaluated whether difference is seen or not.

The obtained results are shown in Table 2. It is understood that CRP can be detected simply by the method of the present invention.

TABLE 2

| | |
|---|---|
| CRP | Detection of Concentration by |
| Concentration | Photothermographic Material |

| (pM) | Sample A | Sample B |
|---|---|---|
| 100 | possible | possible |
| 10 | possible | possible |
| 1 | impossible | possible |

What is claimed is:

1. A method for immunoassaying an object to be analyzed comprising: causing to act, on a specimen or a sample comprising the specimen, a first protein (Y), which is able to specifically recognize the object to be analyzed in the specimen and is modified by a labeling substance, so as to form a complex including the object to be analyzed and Y; and measuring the labeling substance contained in the produced complex or the labeling substance contained in the free protein (Y), wherein the measurement of the labeling substance is carried out by a chemiluminescent method which comprises sensing the light generated by chemiluminescence using a photothermographic material comprising at least an organic silver salt, a reducing agent for silver ions and a photosensitive silver halide to form a latent image, and making the latent image visible by thermal development.

2. The method according to claim 1, wherein the photothermographic material further comprises a nucleator.

3. The method according to claim 1, wherein the photothermographic material further comprises a solvent for the organic silver salt.

4. The method according to claim 1, wherein the organic silver salt comprises a silver salt of a carboxylic acid or a silver salt of a nitrogen-containing heterocyclic compound.

5. The method according to claim 4, wherein a phase transition temperature of the organic silver salt is from 40° C. to 100° C.

6. The method according to claim 1, wherein the labeling substance comprises peroxidase, microperoxidase, glucose-oxidase, alkali phosphatase, β-galactosidase, or luciferase.

7. The method according to claim 1, wherein, in the measurement of the labeling substance, a luminol derivative, a dioxetane derivative, an acridinium derivative, a perbromic acid ester derivative, or a luciferin derivative is used as a chemiluminescent material.

8. The method according to claim 1, wherein the protein which is able to specifically recognize the object to be analyzed comprises an antibody.

9. The method according to claim 8, wherein the antibody comprises a monoclonal antibody.

10. The method according to claim 1, wherein the protein which is able to specifically recognize the object to be analyzed is carried by an insoluble carrier.

11. A method for immunoassaying an object to be analyzed comprising: causing to act, on a specimen or a sample comprising the specimen, a second protein (X), which is able to specifically recognize the object to be analyzed in the specimen, and a first protein (Y), which is able to specifically recognize the object to be analyzed in the specimen and is modified by a labeling substance, simultaneously or stepwise so as to form a complex including X, the object to be analyzed and Y; and measuring the labeling substance contained in the produced complex or the labeling substance contained in the free protein (Y), wherein the measurement of the labeling substance is carried out by a chemiluminescent method which comprises sensing the light generated by chemiluminescence using a photothermographic material comprising at least an organic silver salt, a reducing agent for silver ions and a photosensitive silver halide to form a latent image, and making the latent image visible by thermal development.

12. The method according to claim 11, wherein the photothermographic material further comprises a nucleator.

13. The method according to claim 11, wherein the photothermographic material further comprises a solvent for the organic silver salt.

14. The method according to claim 11, wherein the organic silver salt comprises a silver salt of a carboxylic acid or a silver salt of a nitrogen-containing heterocyclic compound.

15. The method according to claim 14, wherein a phase transition temperature of the organic silver salt is from 40° C. to 100° C.

16. The method according to claim 11, wherein the labeling substance comprises peroxidase, microperoxidase, glucose oxidase, alkali phosphatase, β-galactosidase, or luciferase.

17. The method according to claim 11, wherein, in the measurement of the labeling substance, a luminol derivative, a dioxetane derivative, an acridinium derivative, a perbromic acid ester derivative, or a luciferin derivative is used as a chemiluminescent material.

18. The method according to claim 11, wherein the protein which is able to specifically recognize the object to be analyzed comprises an antibody.

19. The method according to claim 18, wherein the antibody comprises a monoclonal antibody.

20. The method according to claim 11, wherein the protein which is able to specifically recognize the object to be analyzed is carried by an insoluble carrier.

21. An immunoassay method comprising causing to act, on a specimen or a sample comprising the specimen, a second protein (X), which is able to specifically recognize an object to be analyzed in the specimen, and a first protein (Y), which is able to specifically recognize the object to be analyzed in the specimen and is modified by a labeling substance, simultaneously or stepwise so as to form a complex including X, the object to be analyzed and Y₁ wherein the labeling substance is a substance which accelerates development of a thermal developing material comprising at least an organic silver salt and a reducing agent for silver ions, and the complex is detected using the thermal developing material.

22. The immunoassay method according to claim 21, wherein the thermal developing material further comprises a solvent for the organic silver salt.

23. The immunoassay method according to claim 21, wherein the organic silver salt comprises a silver salt of a carboxylic acid or a silver salt of a nitrogen-containing heterocyclic compound.

24. The immunoassay method according to claim 23, wherein a phase transition temperature of the organic silver salt is from 40° C. to 100° C.
